(19) United States
(12) Patent Application Publication
(10) Pub. No.: US 2009/0318389 A1
(43) Pub. Date: Dec. 24, 2009

(54) AGONISTS OF THE SPHINGOSINE-1 PHOSPHATE RECEPTOR

(75) Inventors: Ghotas Evindar, Shrewsbury, MA (US); Hongfeng Deng, Acton, MA (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: PRAECIS PHARMACEUTICALS INCORPORATED, Waltham, MA (US)

(21) Appl. No.: 12/376,141

(22) PCT Filed: Aug. 2, 2007
(86) PCT No.: PCT/US07/17384

§ 371 Date: Mar. 26, 2009

Related U.S. Application Data
(60) Provisional application No. 60/821,432, filed on Aug. 4, 2006.
(60) Provisional application No. 60/821,425, filed on Aug. 4, 2006.
(60) Provisional application No. 60/827,941, filed on Oct. 3, 2006.
(60) Provisional application No. 60/827,928, filed on Oct. 3, 2006.

(51) Int. Cl. A61K 31/675; C07D 277/28; A61K 31/426; C07F 9/6539; C07D 241/04; A61K 31/495

(52) U.S. Cl. 514/92; 548/205; 514/365; 548/119; 544/395; 514/255.03

(57) ABSTRACT

The invention provides compounds formulae I-III, their preparation, and their use as pharmaceutically active immunosuppressive agents for the treatment of autoimmune disorders, organ transplant rejection, disorders associated with an activated immune system, as well as other disorders modulated by lymphopenia or SIP receptors.

### RELATED APPLICATIONS

This application is related and claims priority to U.S. provisional application Ser. No. 60/821,425, filed Aug. 4, 2006; U.S. provisional application Ser. No. 60/827,928, filed Oct. 3, 2006; U.S. provisional application Ser. No. 60/821,432, filed Aug. 4, 2006; and U.S. provisional application Ser. No. 60/827,941, filed Oct. 3, 2006. The entire contents of each of the foregoing applications are incorporated herein by this reference.

### BACKGROUND OF THE INVENTION

Sphingosine 1-phosphate (S1P) is a bioactive sphingolipid metabolite that is secreted by hematopoietic cells and stored and released from activated platelets. S1P is produced by the sphingosine kinase-catalyzed phosphorylation of sphingosine. S1P receptors (S1P-1, -2, -3, -4, and -5) are activated via binding S1P. The receptors are involved in a variety of cellular processes, including cell proliferation and differentiation, cell survival, cell invasion, lymphocyte trafficking, and cell migration.

Administration of S1P to an animal results in sequestration of lymphocytes into the lymph nodes and Peyers patches without causing lymphocyte depletion. This activity, which is of potential utility in treating diseases or conditions associated with inappropriate immune response, such as organ transplant rejection, autoimmune diseases such as multiple sclerosis and rheumatoid arthritis, as well as other disorders modulated by lymphocyte trafficking such as diabetes, hepatitis C (HCV) and cancer, is believed to proceed via activation of the S1P-1 receptor. Administration of S1P in vivo has been shown to cause hypotension and bradycardia, which are believed to be due to signaling through one or more of the other S1P receptors, i.e. S1P-2 to S1P-5. Accordingly, there is a need for compounds which are potent and selective agonists of the S1P-1 receptor.

### SUMMARY OF THE INVENTION

These and other needs are met by the present invention which is directed, at least in part, to a compound of formula I

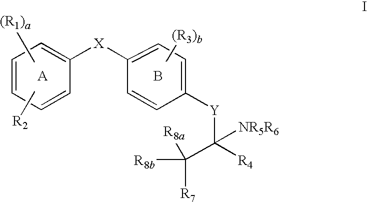

or a pharmaceutically acceptable salt thereof, wherein:

R₁ is halogen, cyano, alkyl, aryl, heteroaryl, cycloalkyl, heterocycloalkyl, aralkyl, heteroalkyl, —O-alkyl, —O-aryl, —O-heteroaryl, —S-alkyl, alkylene-O-alkyl, alkylene-CO₂H, alkylene-CO₂alkyl, alkylSO₂, alkylSO, aralkylSO₂, aralkylSO, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, or dialkylamino, any of which may be optionally substituted on carbon with 1, 2, or 3 groups selected from halo, alkyl, OH, or —O-alkyl;

R₂ is halogen, cyano, alkyl, aryl, heteroaryl, cycloalkyl, heterocycloalkyl, aralkyl, heteroalkyl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylSO₂, alkylSO, aralkylSO₂, aralkylSO, alkylene-CO-amino, alkylene-CO-alkylamino, alkylene-CO-dialkylamino, alkylene-NH—CO₂H, alkylene-NH—CO₂alkyl-CO₂alkyl, —OH, —C(O)-alkyl, —C(O)O-alkyl, —CONH₂, —CO-alkylamino,

—CO-dialkylamino, amino, alkylamino, and dialkylamino, any of which may be optionally substituted on carbon with 1, 2, or 3 groups selected from halo, alkyl, OH, or —O-alkyl;

R₃ is halogen, cyano, alkyl, aryl, heteroaryl, cycloalkyl, heterocycloalkyl, aralkyl, heteroalkyl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

a is 0, 1, 2, or 3;

b is 1, 2 or 3;

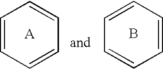

are each independently phenyl or pyridyl;

R⁴ is hydrogen, cyano, alkyl, aryl, heteroaryl, alkylene-OH, alkylene-O-alkyl, —CO₂H, —CO₂-alkyl, alkylene-CO₂H, or alkylene-CO₂-alkyl, alkylene-OC(O)R wherein R is hydrogen or alkyl; cycloalkyl, heterocycloalkyl, alkylene-NH₂, alkylene-alkylamino, or alkylene-dialkylamino, any of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

R₅ and R₆ are each independently selected from the group consisting of hydrogen, alkyl, alkylene-OH, aryl, alkylene-O-alkyl, —CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)-Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or

R₅ and R₆, together with the nitrogen to which they are attached, may form a 3, 4, 5, or 6-membered saturated or unsaturated ring, optionally containing 1 or 2 additional heteroatoms selected from O, S, NH, or N-alkyl, and optionally substituted on carbon with halogen, alkyl, hydroxyl, or alkoxy;

R₇ is selected from the group consisting of alkyl, —OH, —O-alkyl, —CO₂H, —C(O)O-alkyl, —C(O)O-aryl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, —OPO₂(S)NRₚ₁Rₚ₂, and —C(Z′)(Z″)PO₃Rₚ₁Rₚ₂, alkylene-OH, alkylene-CO₂H, alkylene-C(O)O-alkyl, alkylene-C(O)O-aryl, alkylene-CH═CHCO₂H, alkylene-CH₂═CHC(O)O-alkyl, alkylene-CH₂═CHC(O)O-aryl, alkylene-OPO₂Rₚ₁Rₚ₂, -alkylene-OPO₃Rₚ₁Rₚ₂, alkylene-PO₃Rₚ₁Rₚ₂, alkylene-OPO₂(S)Rₚ₁Rₚ₂, and alkylene-C(Z′)(Z″)PO₃Rₚ₁Rₚ₂, any of which may be optionally substituted on carbon with 1, 2, or 3 groups selected from halogen, alkyl, hydroxyl, carboxy, or alkoxy, or any 2 groups on the same carbon may be taken together to from C═O; and wherein

Z′ is hydroxyl or halogen;

Z″ is H or halogen;

Rₚ₁ and Rₚ₂ at each occurrence are independently hydrogen, C₁-C₆-alkyl, or aryl;

X is CRₓ₁Rₓ₂, NRₓ₃, —(CRₓ₁Rₓ₂)ₙNRₓ₃—, —NRₓ₃(CRₓ₁Rₓ₂)ₙ—, —O—, —S—, —(CRₓ₁Rₓ₂)ₙS—, —S(CRₓ₁Rₓ₂)ₙ—, —S(O)—, —(CRₓ₁Rₓ₂)ₙS(O)—, —S(O)(CRₓ₁Rₓ₂)ₙ—, —S(O)₂—, —(CRₓ₁Rₓ₂)ₙS(O)₂—, —S(O)₂(CRₓ₁Rₓ₂)ₙ—, —C(O)—, —(CRₓ₁Rₓ₂)ₙC(O)—, —C(O)(CRₓ₁Rₓ₂)ₙ—, —C(O)O—, —(CRₓ₁Rₓ₂)ₙC(O)O—, and —C(O)O(CRₓ₁Rₓ₂)ₙ—, wherein

Rₓ₁ and Rₓ₂ at each occurrence are independently selected from the group consisting of hydrogen, hydroxyl, halogen, alkyl, —O-alkyl, alkylyele-O-alkyl, alkyl-SO₂, CO₂H, and CO₂-alkyl, any of which may be optionally substituted on carbon with halogen; or taken together Rₓ₁ and Rₓ₂ may form a 3, 4, 5, or 6 membered ring optionally containing 1 or 2 heteroatoms selected from O, S, NH, or N-alkyl, which may itself be substituted on carbon with halogen, hydroxyl, or alkyl;

Rₓ₃ at each occurrence is selected from the group consisting of hydrogen and alkyl;

n is an integer from 0 to 4;

Y is selected from the group consisting of heterocyclo or heteroaryl —CR_{y1}R_{y2}, —CR_{y1}R_{y2}—NR_{y3}—, —NR_{y3}(CO)—, —(CO)—, —O—, —S—, —SO—, —SO₂—, —CR_{y1}R_{y2}—S—, —CR_{y1}R_{y2}—O—, —COO—, and —NR_{y3}SO₂—; wherein

R_{y1}, R_{y2}, R_{y3} at each occurrence are hydrogen or alkyl which may be substituted on carbon with halogen, hydroxyl, or alkyl; or

R_{y3} or —CR_{y1}R_{y2} and one of R₅ or R₆, together with the nitrogens to which they are attached, form a 5, 6, or 7-membered ring, optionally substituted on carbon with halogen, hydroxyl, or alkyl; and

R₈ₐ and R_{8b} are each independently hydrogen, halogen, alkyl, or taken together with the carbon to which they are attached, may form a 3, 4, 5, or 6-membered ring, optionally containing 1 or 2 heteroatoms selected from NH, N-alkyl, O, or S, and optionally substituted on carbon with halogen, or alkyl.

The present invention also provides a compound of formula II

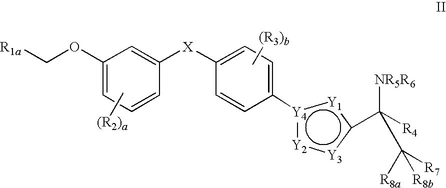

or a pharmaceutically acceptable salt thereof, wherein:

R₁ₐ is aryl or heteroaryl, either of which may be optionally substituted on carbon with 1, 2, or 3 groups selected from halo, alkyl, hydroxyl, or —O-alkyl;

R₂ is halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylSO₂, alkylSO, aralkylSO₂, aralkylSO, alkylene-CO-amino, alkylene-CO-alkylamino, alkylene-CO-dialkylamino, alkylene-NH—CO₂H, alkylene-NH—CO₂alkyl-CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

R₃ is halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl-CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

a is 0, 1, 2, or 3;

b is 1, 2 or 3;

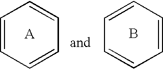

are each independently phenyl or pyridyl;

R₄ is hydrogen, cyano, alkyl, aryl, heteroaryl, alkylene-OH, aryl, alkylene-O-alkyl, —CO₂H, —CO₂-alkyl, alkylene-CO₂H, or alkylene-CO₂-alkyl, alkylene-OC(O)R wherein R is hydrogen or alkyl; cycloalkyl, heterocycloalkyl, alkylene-NH₂, alkylene-alkylamino, or alkylene-dialkylamino, any of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

R₅ and R₆ are each independently selected from the group consisting of hydrogen, alkyl, alkylene-OH, aryl, alkylene-O-alkyl, —CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)-Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or

R₅ and R₆, together with the nitrogen to which they are attached, may form a 3, 4, 5, or 6-membered saturated or unsaturated ring, optionally containing 1 or 2 additional heteroatoms selected from O, S, NH, or N-alkyl, and optionally substituted on carbon with halogen, alkyl, hydroxyl, or alkoxy;

R₇ is selected from the group consisting of alkyl, —OH, —O-alkyl, —CO₂H, —C(O)O-alkyl,

### —C(O)O-aryl, —CH₂═CHCO₂H, —CH₂═CHC(O)O-alkyl, —CH₂═CHC(O)O-aryl, —OPO₂Rₚ₁Rₚ₂,

—OPO₃Rₚ₁Rₚ₂, —CH₂PO₃Rₚ₁Rₚ₂, —OPO₂(S)Rₚ₁Rₚ₂, —OPO₂(S)NRₚ₁Rₚ₂, and —C(Z′)(Z″)PO₃Rₚ₁Rₚ₂, alkylene-OH, alkylene-CO₂H, alkylene-C(O)O-alkyl, alkylene-C(O)O-aryl, alkylene-CH═CHCO₂H, alkylene-CH₂═CHC(O)O-alkyl, alkylene-CH₂═CHC(O)O-aryl, alkylene-OPO₂Rₚ₁Rₚ₂, -alkylene-OPO₃Rₚ₁Rₚ₂, alkylene-PO₃Rₚ₁Rₚ₂, alkylene-OPO₂(S)Rₚ₁Rₚ₂, and alkylene-C(Z′)(Z″)PO₃Rₚ₁Rₚ₂, any of which may be optionally substituted on carbon with 1, 2, or 3 groups selected from halogen, alkyl, hydroxyl, carboxy, or alkoxy, or any 2 groups on the same carbon may be taken together to from C═O; and wherein

Z′ is hydroxyl or halogen;

Z″ is H or halogen;

Rₚ₁ and Rₚ₂ at each occurrence are independently hydrogen, C₁-C₆-alkyl, or aryl;

X is CRₓ₁Rₓ₂, NRₓ₃, —(CRₓ₁Rₓ₂)ₙNRₓ₃—, —NRₓ₃(CRₓ₁Rₓ₂)ₙ—, —O—, —S—, —(CRₓ₁Rₓ₂)ₙS—, —S(CRₓ₁Rₓ₂)ₙ—, —S(O)—, —(CRₓ₁Rₓ₁)ₙS(O)—, —S(O)(CRₓ₁Rₓ₂)ₙ—, —S(O)₂—, —(CRₓ₁Rₓ₂)ₙS(O)₂—, —S(O)₂(CRₓ₁Rₓ₂)ₙ—, —C(O)—, —(CRₓ₁Rₓ₂)ₙC(O)—, —C(O)(CRₓ₁Rₓ₂)ₙ—, —C(O)O—, —(CRₓ₁Rₓ₂)ₙC(O)O—, and —C(O)O(CRₓ₁Rₓ₂)ₙ—; wherein

Rₓ₁ and Rₓ₂ at each occurrence are independently selected from the group consisting of hydrogen, hydroxyl, halogen, alkyl, —O-alkyl, alkylyene-O-alkyl, alkyl-SO₂, CO₂H, and CO₂-alkyl, any of which may be optionally substituted on carbon with halogen; or taken together Rₓ₁ and Rₓ₂ may form a 3, 4, 5, or 6 membered ring optionally containing 1 or 2 heteroatoms selected from O, S, NH, or N-alkyl, which may itself be substituted on carbon with halogen, hydroxyl, or alkyl;

Rₓ₃ at each occurrence is selected from the group consisting of hydrogen and alkyl;

n is an integer from 0 to 4; and

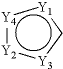

is a heteroaryl ring containing up to four heteroatoms selected from N, O, or S, optionally substituted on carbon with halogen or alkyl, wherein

Y₁ is N, S, or O;

Y₂ and Y₃ are each independently C, N, O, or S; provided that when

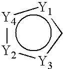

contains an N—H, that hydrogen may be replaced with alkyl;

Y₄ is C or N; and

R₈ₐ and R_{8b} are each independently hydrogen, halogen, alkyl, or taken together with the carbon to which they are attached, may form a 3, 4, 5, or 6-membered ring, optionally containing 1 or 2 heteroatoms selected from NH, N-alkyl, O, or S, and optionally substituted on carbon with halogen, or alkyl

The present invention also provides a compound of formula III

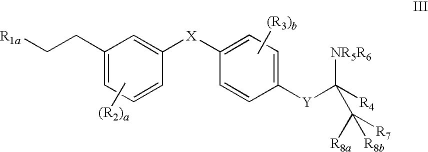

or a pharmaceutically acceptable salt thereof, wherein:

R₁ₐ is aryl or heteroaryl, either of which may be optionally substituted on carbon with 1, 2, or 3 groups selected from halo, alkyl, hydroxyl, or —O-alkyl;

R₂ is halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylSO₂, alkylSO, aralkylSO₂, aralkylSO, alkylene-CO-amino, alkylene-CO-alkylamino, alkylene-CO-dialkylamino, alkylene-NH—CO₂H, alkylene-NH—CO₂alkyl-CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

R₃ is halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl-CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

a is 0, 1, 2, or 3;

b is 1, 2, or 3;

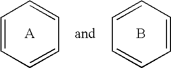

are each independently phenyl or pyridyl;

R₄ is hydrogen, cyano, alkyl, aryl, heteroaryl, alkylene-OH, aryl, alkylene-O-alkyl, —CO₂H, —CO₂-alkyl, alkylene-CO₂H, or alkylene-CO₂-alkyl, alkylene-OC(O)R wherein R is hydrogen or alkyl; cycloalkyl, heterocycloalkyl, alkylene-NH₂, alkylene-alkylamino, or alkylene-dialkylamino, any of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

R₅ and R₆ are each independently selected from the group consisting of hydrogen, alkyl, alkylene-OH, aryl, alkylene-O-alkyl, —CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)—Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or

R₅ and R₆, together with the nitrogen to which they are attached, may form a 3, 4, 5, or 6-membered saturated or unsaturated ring, optionally containing 1 or 2 additional heteroatoms selected from O, S, NH, or N-alkyl, and optionally substituted on carbon with halogen, alkyl, hydroxyl, or alkoxy;

R₇ is selected from the group consisting of alkyl, —OH, —O-alkyl, —CO₂H, —C(O)O-alkyl, —C(O)O-aryl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, —OPO₂(S)NRₚ₁Rₚ₂, and —C(Z′)(Z″)PO₃Rₚ₁Rₚ₂, alkylene-OH, alkylene-CO₂H, alkylene-C(O)O-alkyl, alkylene-C(O)O-aryl, alkylene-CH═CHCO₂H, alkylene-CH₂═CHC(O)O-alkyl, alkylene-CH₂═CHC(O)O-aryl, alkylene-OPO₂Rₚ₁Rₚ₂, -alkylene-OPO₃Rₚ₁Rₚ₂, alkylene-PO₃Rₚ₁Rₚ₂, alkylene-OPO₂(S)Rₚ₁Rₚ₂, and alkylene-C(Z′)(Z″)PO₃Rₚ₁Rₚ₂, any of which may be optionally substituted on carbon with 1, 2, or 3 groups selected from halogen, alkyl, hydroxyl, carboxy, or alkoxy, or any 2 groups on the same carbon may be taken together to from C═O; and wherein

Z′ is hydroxyl or halogen;

Z″ is H or halogen;

Rₚ₁ and Rₚ₂ at each occurrence are independently hydrogen, C₁-C₆-alkyl, or aryl;

X is CRₓ₁Rₓ₂, NRₓ₃, —(CRₓ₁Rₓ₂)ₙNRₓ₃—, —NRₓ₃(CRₓ₁Rₓ₂)ₙ—, —O—, —S—, —(CRₓ₁Rₓ₂)ₙS—, —S(CRₓ₁Rₓ₂)ₙ—, —S(O)—, —(CRₓ₁Rₓ₂)ₙS(O)—, —S(O)(CRₓ₁Rₓ₂)ₙ—, —S(O)₂—, —(CRₓ₁Rₓ₂)ₙS(O)₂—, —S(O)₂(CRₓ₁Rₓ₂)ₙ—, —C(O)—, —(CRₓ₁Rₓ₂)ₙC(O)—, —C(O)(CRₓ₁Rₓ₂)ₙ—, —C(O)O—, —(CRₓ₁Rₓ₂)ₙC(O)O—, and —C(O)O(CRₓ₁Rₓ₂)ₙ—; wherein

Rₓ₁ and Rₓ₂ at each occurrence are independently selected from the group consisting of hydrogen, hydroxyl, halogen, alkyl, —O-alkyl, alkylyene-O-alkyl, alkyl-SO₂, CO₂H, and CO₂-alkyl, any of which may be optionally substituted on carbon with halogen; or taken together Rₓ₁ and Rₓ₂ may form a 3, 4, 5, or 6 membered ring optionally containing 1 or 2 heteroatoms selected from O, S, NH, or N-alkyl, which may itself be substituted on carbon with halogen, hydroxyl, or alkyl;

Rₓ₃ at each occurrence is selected from the group consisting of hydrogen and alkyl;

n is an integer from 0 to 4; and

Y is —CH₂NR′″—, —CH₂NR′″(CO—, —CHFNR′″—, —CHFNR′″(CO)—, —CF₂NR′″—, —CF₂NR′″(CO)—, —CH₂(CO)—, —CHF(CO)—, —CF₂(CO)—, —(CO)CF₂—, —CH₂(CHOH)—, —CHF(CHOH)—, —CF₂(CHOH)—, —(CHOH)CF₂—, —NH(CO)—, —(CO)—, —(CO)₂—, —O—, —S—, —SO—, —SO₂—, —CH₂O—, —CH₂CH₂O—, —CH₂OCH₂—, —OCH₂O—, —CH₂S—, —CH₂SO—, —CH₂SO₂—, —CHFO—, —CHFS—, —CHFSO—, —CHFSO₂—, —CF₂O—, —CF₂S—, —CF₂SO—, —CF₂SO₂—, —NR′″SO₂—, —CF₂—, —CF₂CF₂—, —CF₂CF₂CF₂—, wherein R′″ is H or alkyl; and

R₈ₐ and R_{8b} are each independently hydrogen, halogen, alkyl, or taken together with the carbon to which they are attached, may form a 3, 4, 5, or 6-membered ring, optionally containing 1 or 2 heteroatoms selected from NH, N-alkyl, O, or S, and optionally substituted on carbon with halogen, or alkyl.

The present invention is also directed to a compound which is:

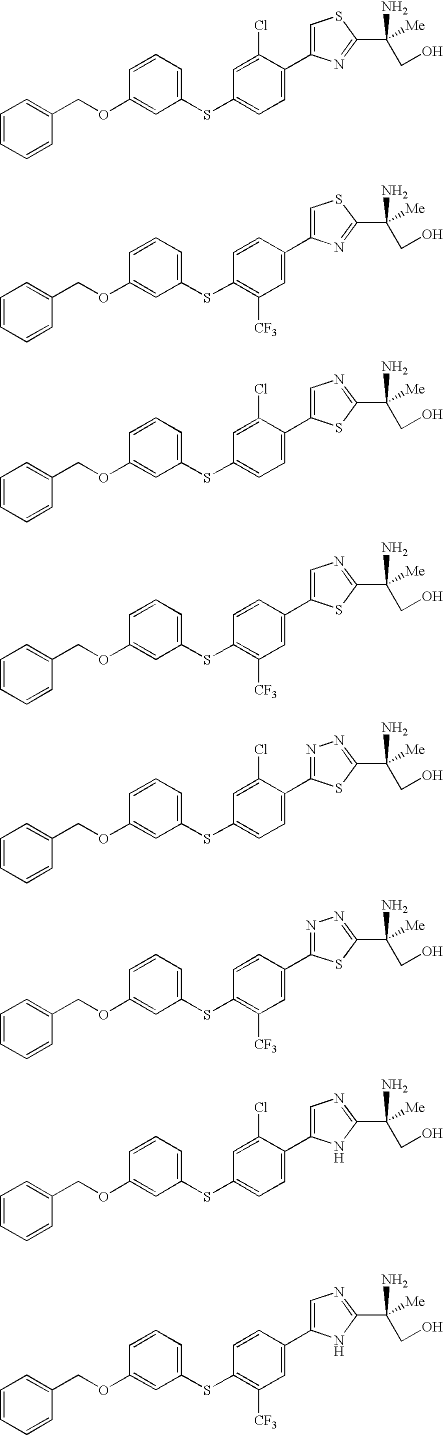

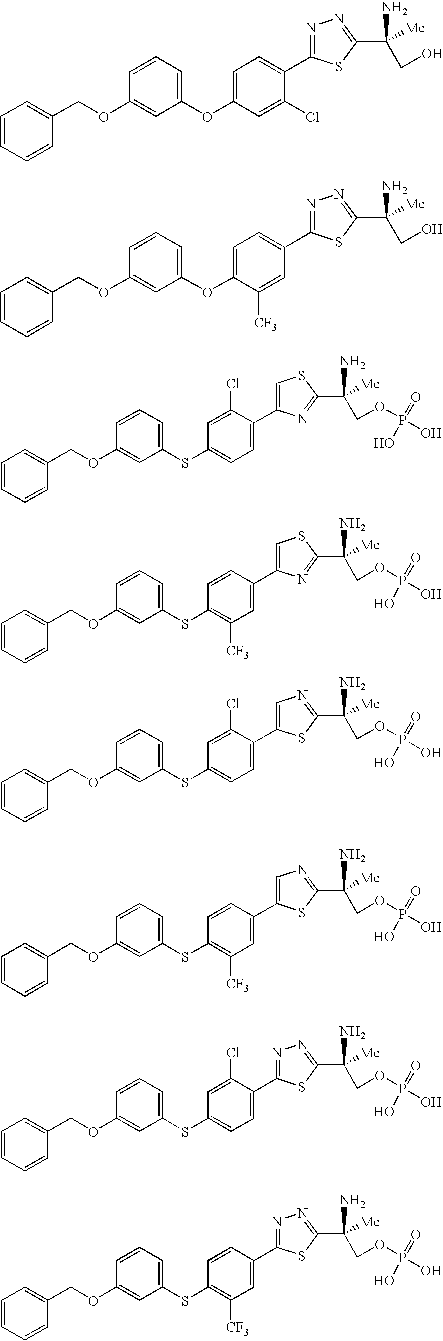

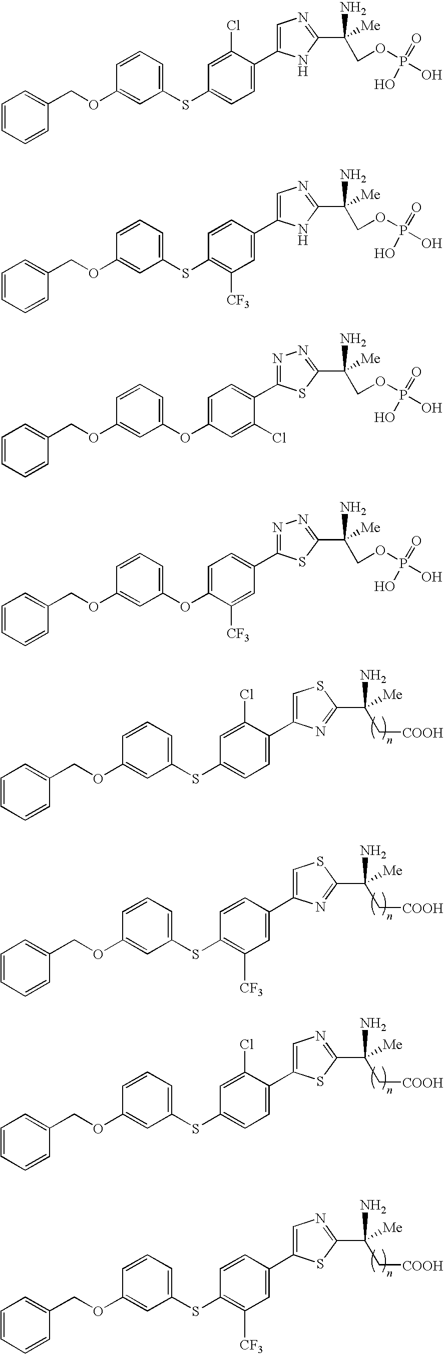

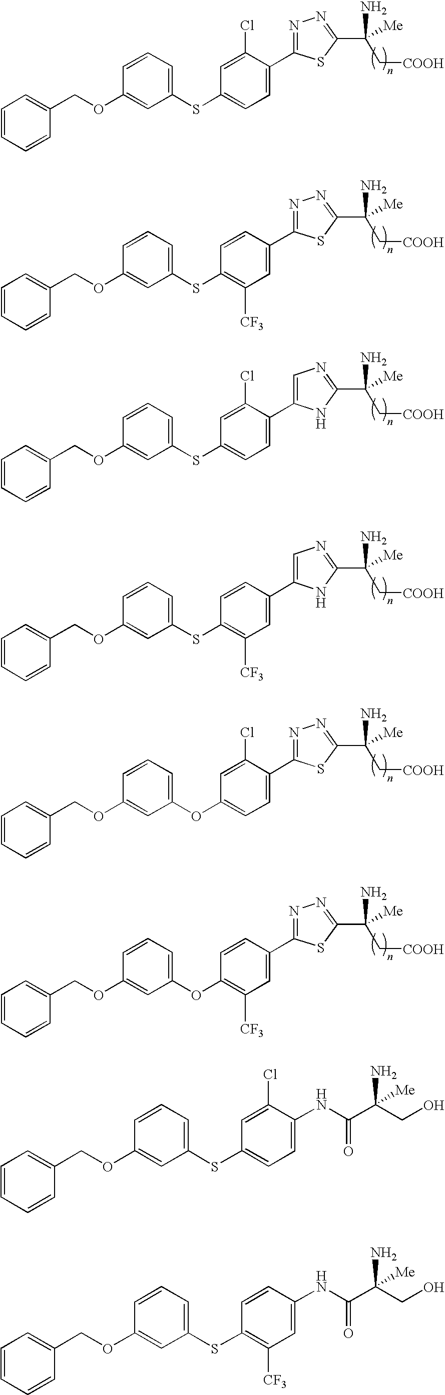

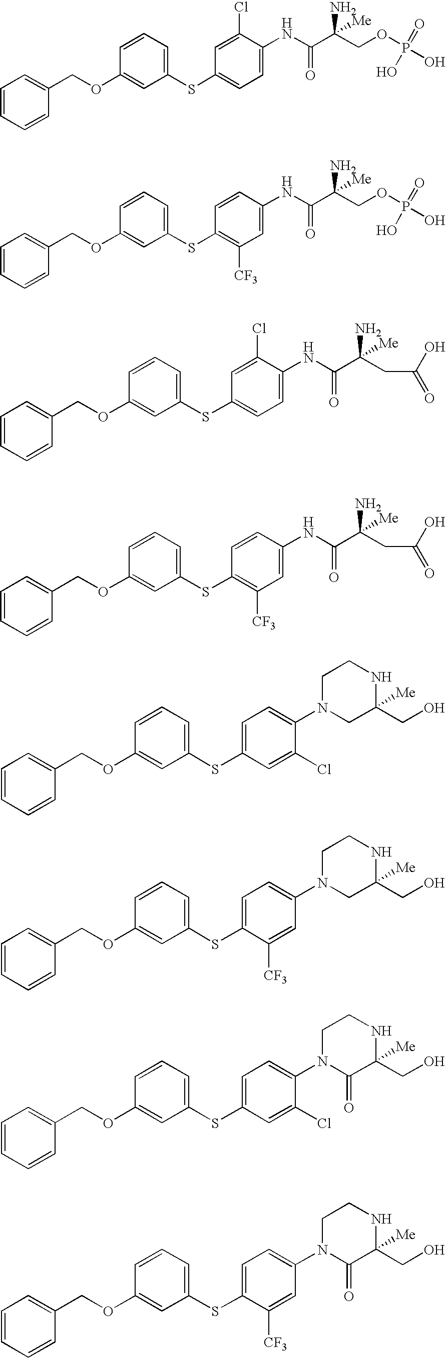

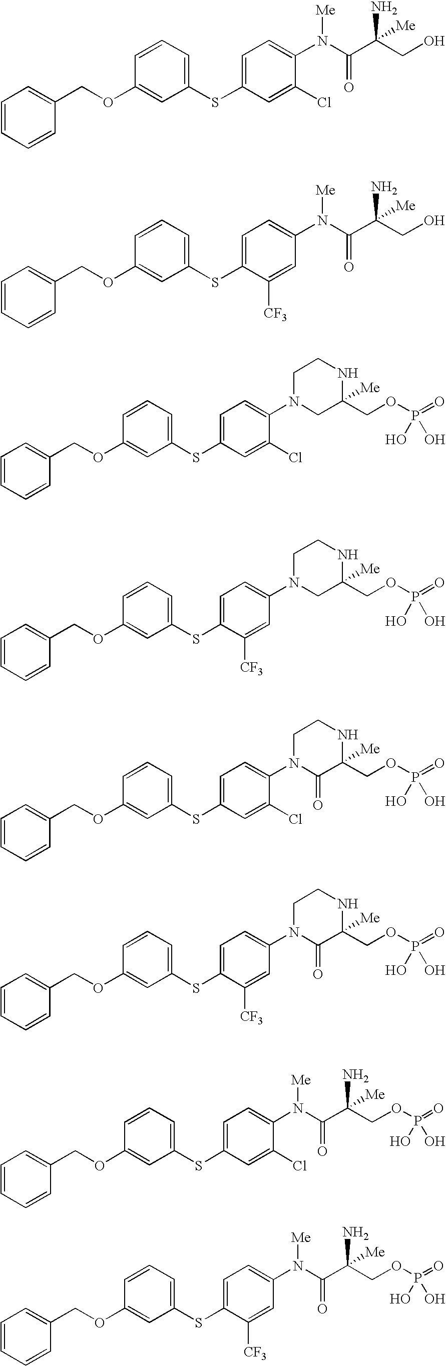

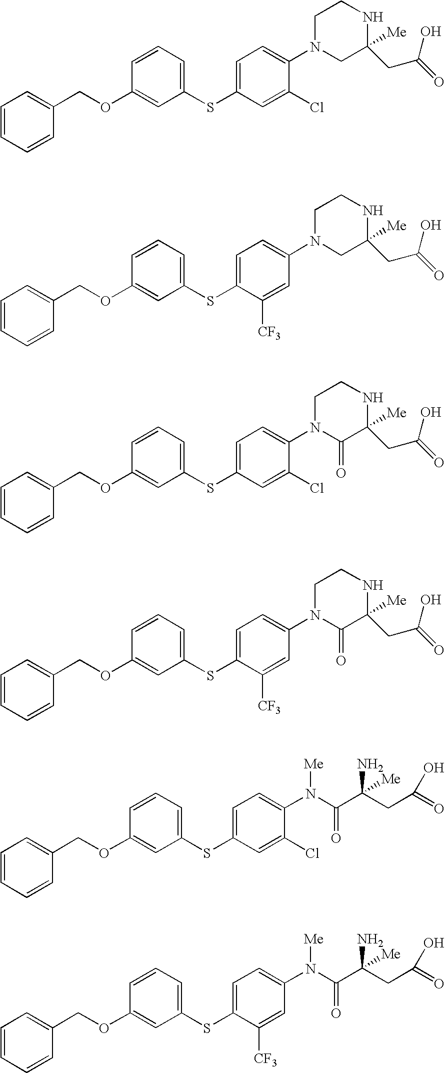

wherein n is 0, 1, or 2 for the above compounds, as well as pharmaceutically acceptable salts, phosphate derivatives, phosphate mimics, or phosphate precursor analogs thereof.

The invention is also directed to a method of treating an autoimmune disorder, comprising administering to a subject in need thereof a pharmaceutically acceptable amount of any of the compounds described herein, e.g., compounds of formulae I-III.

The invention is also directed to a method treating transplant rejection comprising administering to a subject in need thereof a pharmaceutically acceptable amount of any of the compounds described herein, e.g., compounds of formulae I-III.

The invention is also directed to a method treating multiple sclerosis comprising administering to a subject in need thereof a pharmaceutically acceptable amount of any of the compounds described herein, e.g., compounds of formulae I-III.

The invention is also directed to a method treating asthma comprising administering to a subject in need thereof a pharmaceutically acceptable amount of any of the compounds described herein, e.g., compounds of formulae I-III.

The invention is also directed to a method treating rheumatoid arthritis comprising administering to a subject in heed thereof a pharmaceutically acceptable amount of any of the compounds described herein, e.g., compounds of formulae I-III.

The invention is also directed to a method treating cancer comprising, administering to a subject in need thereof a pharmaceutically acceptable amount of any of the compounds described herein, e.g., compounds of formulae I-III.

The invention is also directed to a method treating hepatitis C (HCV) comprising, administering to a subject in need thereof a pharmaceutically acceptable amount of any of the compounds described herein, e.g., compounds of formulae I-III.

The invention is also directed to a method treating diabetes comprising, administering to a subject in need thereof a pharmaceutically acceptable amount of any of the compounds described herein, e.g., compounds of formulae I-III.

The invention is also directed to a pharmaceutical composition, comprising any of the compounds described herein, e.g., compounds of formulae I-III and a pharmaceutically acceptable carrier.

The invention is also directed to a process for making any of the compounds described herein, e.g., compounds of formulae I-III, e.g., a method as provided in Schemes 1, 2, 3 and 4.

### BRIEF DESCRIPTION OF THE DRAWING

FIG. 1 is a graph showing the results of the lymphopenia assay for certain compounds of the invention.

### DETAILED DESCRIPTION OF THE INVENTION

### Definitions

The following definitions are used, unless otherwise described.

“Halogen” or “halo” means fluoro (F), chloro (Cl), bromo (Br), or iodo (I).

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to monovalent straight or branched chain hydrocarbon radicals comprising 1 to about 12 carbon atoms.

The term “alkylene” used alone or as suffix or prefix, refers to divalent straight or branched chain hydrocarbon radicals comprising 1 to about 12 carbon atoms, which serves to links two structures together.

The term “cycloalkyl” used alone or as suffix or prefix, refers to a saturated or partially unsaturated monovalent ring-containing hydrocarbon radical comprising at least 3 up to about 12 carbon atoms.

The term “aryl” used alone or as suffix or prefix, refers to a monovalent hydrocarbon radical having one or more polyunsaturated carbon rings having aromatic character, and comprising 5 up to about 14 carbon atoms.

The term “heterocycle” used alone or as a suffix or prefix, refers to a ring-containing structure or molecule having one or more multivalent heteroatoms, independently selected from N, O and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The terms “heterocyclic group”, “heterocyclic moiety”, “heterocyclic”, or “heterocyclo” used alone or as a suffix or prefix, refers to a radical derived from a heterocycle by removing one or more hydrogens therefrom.

The term “heterocyclyl” used alone or as a suffix or prefix, refers a monovalent radical derived from a heterocycle by removing one hydrogen therefrom.

The term “heteroaryl” used alone or as a suffix or prefix, refers to a heterocyclyl having aromatic character.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition, heterocycle includes aromatic heterocycles (heteroaryl groups), for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocycle includes

polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

Heterocyclyl includes, for example, monocyclic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydropyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “six-membered” used as prefix refers to a group having a ring that contains six ring atoms.

The term “five-membered” used as prefix refers to a group having a ring that contains five ring atoms.

A five-membered heteroaryl ring is a heteroaryl with a ring having five ring atoms wherein 1, 2 or 3 ring atoms are independently selected from N, O and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S. Exemplary six-membered ring heteroaryls are pyridyl, pyrazinyl, pyrimidinyl, triazinyl and pyridazinyl.

The term “aralkyl” refers to an alkyl group substituted with an aryl group.

The term “heteroaralkyl” refers to an alkyl group substituted with an heteroaryl group.

Unless otherwise specified, the term “substituted”, when used as a prefix, refers to a structure, molecule or group, wherein one or more hydrogens are replaced with one or more alkyl groups, or one or more chemical groups containing one or more heteroatoms selected from N, O, S, F, Cl, Br, I, and P. Exemplary chemical groups containing one or more heteroatoms include heterocyclyl, —NO₂, —O-alkyl, halo, —CF₃, —CO₂H, —CO₂R, —NH₂, —SH, —NHR, —NR₂, —SR, —SO₃H, —SO₂R, —S(O)R, —CN, —OH, —C(O)NR₂, —NRC(O)R, oxo (═O), imino (═NR), thio (═S), and oximino (═N—OR), wherein each “R” is alkyl as defined above. For example, substituted phenyl may refer to nitrophenyl, pyridylphenyl, methoxyphenyl, chlorophenyl, aminophenyl, and so on, wherein the nitro, pyridyl, methoxy, chloro, and amino groups may replace any suitable hydrogen on the phenyl ring.

The term “alkoxy” used alone or as a suffix or prefix, refers to radicals of the general —O-alkyl, Exemplary alkoxy groups includes methoxy, ethoxy, propoxy, isopropoxy, butoxy, t-butoxy, isobutoxy, cyclopropylnethoxy, allyloxy, and propargyloxy.

The term “amine” or “amino” used alone or as a suffix or prefix, refers —NH₂.

The term “alkylamino” used alone or as a suffix or prefix, refers —NH(alkyl).

The term “dialkylamino” used alone or as a suffix or prefix, refers —N(alkyl)₂.

“Acyl” used alone, as a prefix or suffix, means —C(O)—R, wherein R hydrogen, hydroxyl, amino, alkylamino, dialkylamino, or alkoxy, any of which may be substituted as provided by the definition of “substituted” given above. Acyl groups include, for example, acetyl, propionyl, benzoyl, phenyl acetyl, carboethoxy, and dimethylcarbamoyl.

Some of the compounds in the present invention may exist as stereoisomers, including enantiomers, diastereomers, and geometric isomers. All of these forms, including (R), (S), epimers, diastereomers, cis, trans, syn, anti, solvates (including hydrates), tautomers, and mixtures thereof, are contemplated in the compounds of the present invention.

The invention also relates to salts of the compounds of the invention and, in particular, to pharmaceutically acceptable salts. A “pharmaceutically acceptable salt” is a salt that retains the desired biological activity of the parent compound and does not impart any undesired toxicological effects. The salts can be, for example, salts with a suitable acid, such as hydrochloric acid, hydrobromic acid, sulfuric acid, phosphoric acid, nitric acid, and the like; acetic acid, oxalic acid, tartaric acid, succinic acid, malic acid, benzoic acid, pamoic acid, alginic acid, methanesulfonic acid, naphthalenesulfonic acid, and the like. Also included are salts of cations such as ammonium, sodium, potassium, lithium, zinc, copper, barium, bismuth, calcium, and the like; or organic cations such as tetralkylammonium and trialkylammonium cations. Combinations of the above salts are also useful. Salts of other acids and/or cations are also included, such as salts with trifluoroacetic acid, chloroacetic acid, and trichloroacetic acid. The invention also includes different crystal forms, hydrates, and solvates of the compounds of the invention.

The terms “phosphate precursor” and “phosphate precursor analog,” as used herein, refer to substituent moieties in invention compounds that may be directly phosphorylated in vivo, or which may be cleaved in vivo to reveal a moiety that may then be phosphorylated in vivo. In certain embodiments, the phosphate precursor may be L₁-O—H or L₁-O-L₂, wherein L₁ is a linking moiety and L₂ is a labile moiety. Exemplary embodiments of the phosphate precursor, include but are not limited to -alkyl-OH, -halo-alkyl-OH, alkoxy-OH, -alkyl-OCOR^{a}, -halo-alkyl-OCOR^{a}, -alkoxy-OCOR^{a}, -alkyl-OC(O)NR^{a}R^{b}, -halo-alkyl-OC(O)NHR^{a}R^{b}, -alkoxy-OC(O)NR^{a}R^{b}, —(CH₂)_{q}CO₂R^{c}, and —(CH₂)ₙCH₂═CHC(O)OR^{c}, wherein

q is an integer between 0 and 4;

R^{a} and R^{b} are independently selected from the group consisting of hydrogen, straight chain or branched C₁-C₆-alkyl, all of which may be optionally substituted with OH, halogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; and

R^{c} is selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

The “linking moiety,” may contain 1-8 atoms or may be a bond, and serves as the connection point through which the phosphate mimic, phosphate derivative, or phosphate precursor substituent moieties are linked to the remaining structure of the compounds of the invention. In certain embodiments, the linking moiety may include, but is not limited to, substituted or unsubstituted alkyl (e.g., methylene chains), substituted or unsubstituted alkenyl (e.g., n-alkenes), substituted or unsubstituted alkynyl, substituted or unsubstituted halo-alkyl, substituted or unsubstituted alkoxy, and substituted or unsubstituted halo-alkoxy. In specific embodiments, the linking moiety may be carbonyl derivatized.

The language “labile moiety” refers to a moiety that is subject to cleavage, for instance, by hydrolysis or enzymatic degradation. In certain embodiments, the labile moiety is an ester moiety, which may result in a carboxylate or hydroxyl derivative, depending on the orientation of the ester functionality in the molecule prior to cleavage.

The term “prodrug derivatizing moiety (PDM)” refers to a moiety that derivatizes the compounds of the invention, resulting in a prodrug. Such prodrugs are art-recognized, and are often used to mask particular functionalities that are excessively reactive in vivo. In certain embodiments the PDM is selected from the group consisting of:

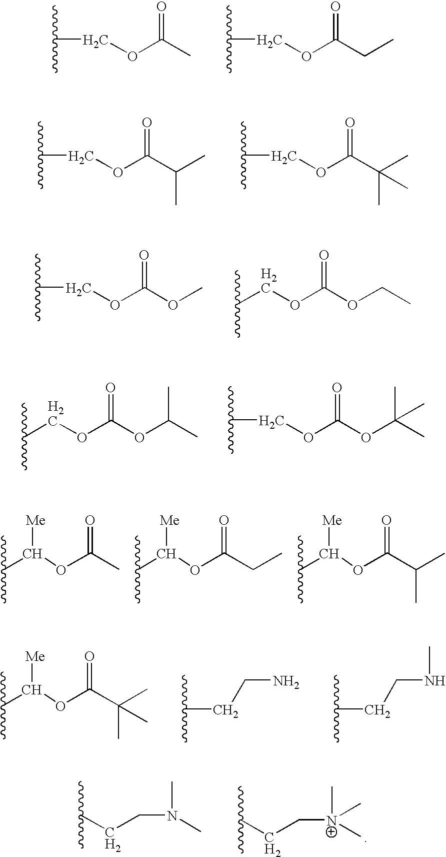

The term “phosphate derivative” refers to substituent moieties in invention compounds that contain a phosphate or phosphate ester group. When a compound of the invention containing a phosphate derivative is administered to a subject, the compound may act as is in vivo or the phosphate derivative (within the compound) may be cleaved and then re-phosphorylated in vivo leading to an active compound. In certain embodiments, the phosphate derivative may be selected from the group consisting of —(CH₂)_{q}OPO₂R^{d}R^{e}, —(CH₂)_{q}OPO₃R^{d}R^{e}, and —(CH₂)_{q}OPO₂(S)R^{d}R^{e}, wherein

q is an integer between 0 and 4; and

R^{d} and R^{e} are each independently selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

The term “phosphate mimic” refers to substituent moieties in invention compounds in which a phosphate substrate has been replaced with a non-hydrolyzable functional group, resulting in a moiety that mimics the structural and/or electronic attributes of a phosphate or phosphate ester moiety. In certain embodiments, the phosphate mimic is -L₁-Z₂, wherein L₁ is a linking moiety and Z₂ is a non-hydrolyzable moiety covalently bonded, to L₁. In certain embodiments, the phosphate mimic is selected from the group consisting of

—(CH₂)_{q}CH₂PO₃R^{d}R^{e}, and —(CH₂)_{q}C(Y₁)(Y₂)PO₃R^{d}R^{e}, wherein

q is an integer between 0 and 4;

Y₁ and Y₂ are independently selected from the group consisting of hydrogen, straight chain or branched C₁-C₆-alkyl, all of which may be optionally substituted with OH, halogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; and

R^{d} and R^{e} are each independently selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

The language “non-hydrolyzable moiety” is art-recognized, and refers to moieties containing bonds, such as carbon-phosphorous bonds, that are not hydrolyzable in vivo.

### Invention Compounds

In some aspects, the present invention is drawn to a compound of formula I.

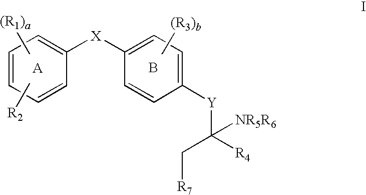

or a pharmaceutically acceptable salt thereof, wherein:

R₁ is halogen, cyano, alkyl, aryl, heteroaryl, cycloalkyl, heterocycloalkyl, aralkyl, heteroalkyl, —O-alkyl, —O-aryl, —O-heteroaryl, —S-alkyl, alkylene-O-alkyl, alkylene-CO₂H, alkylene-CO₂alkyl, alkylSO₂, alkylSO, aralkylSO₂, aralkylSO, alkylene-CO-amino, alkylene-CO-alkylamino, alkylene-CO-dialkylamino, alkylene-NH—CO₂H, alkylene-NH—CO₂alkyl-CO₂alkyl, —OH, —C(O)-alkyl, —C(O)O-alkyl, —CONH₂, —CO-alkylamino, —CO-dialkylamino, amino, alkylamino, or dialkylamino, any of which may be optionally substituted on carbon with 1, 2, or 3 groups selected from halo, alkyl, OH, or —O-alkyl;

R₂ is halogen, cyano, alkyl, aryl, heteroaryl, cycloalkyl, heterocycloalkyl, aralkyl, heteroalkyl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylSO₂, alkylSO, aralkylSO₂, aralkylSO, alkylene-CO-amino, alkylene-CO-alkylamino, alkylene-CO-dialkylamino, alkylene-NH—CO₂H, alkylene-NH—CO₂alkyl-CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

R₃ is halogen, cyano, alkyl, aryl, heteroaryl, cycloalkyl, heterocycloalkyl, aralkyl, heteroalkyl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl-CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

a is 0, 1, 2, or 3;

b is 1, 2 or 3;

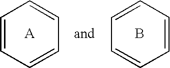

are each independently phenyl or pyridyl;

R₄ is hydrogen, cyano, alkyl, aryl, heteroaryl, alkylene-OH, alkylene-O-alkyl, —CO₂H, —CO₂-alkyl, alkylene-CO₂H, or alkylene-CO₂-alkyl, alkylene-OC(O)R wherein R is hydrogen or alkyl; cycloalkyl, heterocycloalkyl, alkylene-NH₂, alkylene-alkylamino, or alkylene-dialkylamino, any of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

R₅ and R₆ are each independently selected from the group consisting of hydrogen, alkyl, alkylene-OH, aryl, alkylene-O-alkyl,—CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)—Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or

R₅ and R₆, together with the nitrogen to which they are attached, may form a 3, 4, 5, or 6-membered saturated or unsaturated ring, optionally containing 1 or 2 additional heteroatoms selected from O, S, NH, or N-alkyl, and optionally substituted on carbon with halogen, alkyl, hydroxyl, or alkoxy;

R₇ is selected from the group consisting of alkyl, —OH, —O-alkyl, —CO₂H, —C(O)O-alkyl, —C(O)O-aryl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, —OPO₂(S)NRₚ₁Rₚ₂, and —C(Z′)(Z″)PO₃Rₚ₁Rₚ₂, alkylene-OH, alkylene-CO₂H, alkylene-C(O)O-alkyl, alkylene-C(O)O-aryl, alkylene-CH═CHCO₂H, alkylene-CH₂═CHC(O)O-alkyl, alkylene-CH₂═CHC(O)O-aryl, alkylene-OPO₂Rₚ₁Rₚ₂, -alkylene-OPO₃Rₚ₁Rₚ₂, alkylene-PO₃Rₚ₁Rₚ₂, alkylene-OPO₂(S)Rₚ₁Rₚ₂, and alkylene-C(Z′)(Z″)PO₃Rₚ₁Rₚ₂, any of which may be optionally substituted on carbon with 1, 2, or 3 groups selected from halogen, alkyl, hydroxyl, carboxy, or alkoxy, or any 2 groups on the same carbon may be taken together to from C═O; and wherein

Z′ is hydroxyl or halogen;

Z″ is H or halogen;

Rₚ₁ and Rₚ₂ at each occurrence are independently hydrogen, C₁-C₆-alkyl, or aryl;

X is CRₓ₁Rₓ₂, NRₓ₃, —(CRₓ₁Rₓ₂)ₙNRₓ₃—, —NRₓ₃(CRₓ₁Rₓ₂)ₙ—, —O—, —S—, —(CRₓ₁Rₓ₂)ₙS—, —S(CRₓ₁Rₓ₂)ₙ—, —S(O)—, —(CRₓ₁Rₓ₂)ₙS(O)—, —S(O)(CRₓ₁Rₓ₂)ₙ—, —S(O)₂—, —(CRₓ₁Rₓ₂)ₙS(O)₂—, —S(O)₂(CRₓ₁Rₓ₂)ₙ—, —C(O)—, —(CRₓ₁Rₓ₂)ₙC(O)—, —C(O)(CRₓ₁Rₓ₂)ₙ—, —C(O)O—, —(CRₓ₁Rₓ₂)ₙC(O)O—, and —C(O)O(CRₓ₁Rₓ₂)ₙ—; wherein

Rₓ₁ and Rₓ₂ at each occurrence are independently selected from the group consisting of hydrogen, hydroxyl, halogen, alkyl, —O-alkyl, alkylyene-O-alkyl, alkyl-SO₂, CO₂H, and CO₂-alkyl, any of which may be optionally substituted on carbon with halogen; or taken together Rₓ₁ and Rₓ₂ may form a 3, 4, 5, or 6 membered ring optionally containing 1 or 2 heteroatoms selected from O, S, NH, or N-alkyl, which may itself be substituted on carbon with halogen, hydroxyl, or alkyl;

Rₓ₃ at each occurrence is selected from the group consisting of hydrogen and alkyl;

n is an integer from 0 to 4;

Y is selected from the group consisting of heterocyclo or heteroaryl-CR_{y1}R_{y2}, —CR_{y1}R_{y2}—NR_{y3}—, —NR_{y3}(CO)—, —(CO)—, —O—, —S—, —SO—, —SO₂—, —CR_{y1}R_{y2}—S—, —CR_{y1}R_{y2}—O—, —COO—, and —NR_{y3}SO₂—; wherein

R_{y1}, R_{y2}, R_{y3} at each occurrence are hydrogen or alkyl which may be substituted on carbon with halogen, hydroxyl, or alkyl; or

R_{y3} or —CR_{y1}R_{y2} and one of R₅ or R₆, together with the nitrogens to which they are attached, form a 5, 6, or 7-membered ring, optionally substituted on carbon with halogen, hydroxyl, or alkyl; and

R₈ₐ and R_{8b} are each independently hydrogen, halogen, alkyl, or taken together with the carbon to which they are attached, may form a 3, 4, 5, or 6-membered ring, optionally containing 1 or 2 heteroatoms selected from NH, N-alkyl, O, or S, and optionally substituted on carbon with halogen, or alkyl.

It is to be understood that when the values for a and b are less than the total number of open substituents on the ring, the remainder of the substituents are hydrogen.

That is, if b is 1, the remaining three substituents on

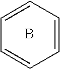

are hydrogen.

In some embodiments, R₁ is benzyloxy.

The compounds of the present invention include a selectivity enhancing moiety. The term “selectivity enhancing moiety (SEM)” is defined in U.S. application Ser. No. 11/349069 filed on Feb. 6, 2006 which is assigned to the assignee of the present application, the contents of which are incorporated herein by reference, refers to one or more moieties that provide an enhancement in the selectivity of the compound to which they are attached for the S1P-1 receptor, as compared to the compound not containing the moiety or moieties. The SEM confers selectivity to the compound to which it is attached for the S1P-1 receptor as compared to, for example, the S1P-2 to S1P-5 receptors. The enhancement conferred to a compound by the SEM may be measured by, for example, determining the binding specificity of a compound for the S1P-1 receptor and one or more of the other S1P receptors. The enhancement conferred to a compound by the SEM may be in the form of increased potency for S1P-1 or decreased potency for any one of S1P-2 through S1P-5. The SEM of the present application is defined in one embodiment as for R₂ and R₃.

The SEM may be a halogen such as F or Cl. It may also be a halo-substituted alkyl group such as CF₃, CF₂CF₃, CF₂CF₂CF₃, CFHCF₃, CH₂CF₃, CH₂CH₂CF₃, CHCl₂, or CH₂Cl. It may also be cyano.

In certain embodiments, the SEM may possess a selectivity enhancing orientation (SEO). The term “selectivity enhancing orientation” or “SEO,” is defined in U.S. application Ser. No. 11/349069 filed on Feb. 6, 2006 which is assigned to the assignee of the present application, the contents of which are incorporated herein by reference and as used herein refers to the relative selectivity enhancement of a compound based on the orientation of the SEM as well as the additional substituents on the ring, either alone or in combination with each other. In particular, the SEO may result from the orientation of the SEM on the ring to which it is attached, in relation to any other ring and/or moiety attached to the same ring. In one embodiment, the SEM on

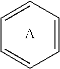

is in the ortho position relative to X in Formula I. In another specific embodiment, the SEM is in the meta position relative to X.

Thus, a specific value for R₃ is trifluoromethyl. Another specific value for R₃ is fluoro. Another specific value for R₃ is chloro. Another specific value for R₃ is bromo, Another specific value for R₃ is cyano. Another specific value for R₃ is methyl. Another specific value for R₃ is dimethylamino.

In some embodiments, a is 0. In other embodiments, a is 1.

In some embodiments, b is 1.

In some embodiments,

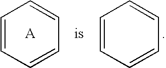

In other embodiments,

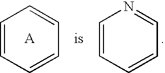

In some embodiments,

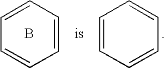

In other embodiments,

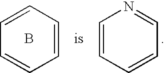

In some embodiments, R₄ is hydrogen. In other embodiments, R₄ is methyl. In other embodiments, R₄ is hydroxymethyl.

In some embodiments, R₅ and R₆ are independently hydrogen.

In some embodiments, R₇ is OH. In other embodiments, R₇ is CO₂H. In other embodiments, R₇ is CO₂Me or CO₂Et. Another In other embodiments, R₇ is CO₂-phenyl. In other embodiments, R₇ is —OP(O)₃H₂.

In some embodiments, X is CH₂. In other embodiments, X is NH or N-alkyl. In other embodiments, X is O. In other embodiments, X is S, SO, or SO₂. In other embodiments, X is CO.

In some embodiments, Y is

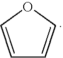

In other embodiments, Y is

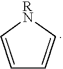

In other embodiments, Y is

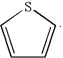

In other embodiments, Y is

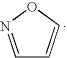

In other embodiments, Y is

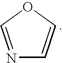

In some embodiments, Y is

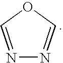

In other embodiments, Y is

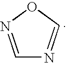

In other embodiments, Y is

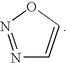

In other embodiments, Y is

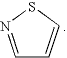

some embodiments, Y is

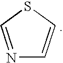

In other embodiments, Y is

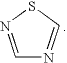

In other embodiments, Y is

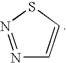

In other embodiments, Y is

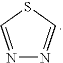

Another specific value for Y is

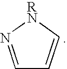

In some embodiments,

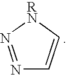

In other embodiments, Y is

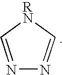

In other embodiments, Y is

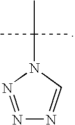

In other embodiments, Y is

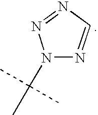

In the above structures, R can be hydrogen or alkyl, and

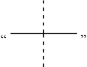

indicates a point of attachment.

In some embodiments, Y is CH₂. In other embodiments, Y is CH₂NH. In other embodiments, Y is NH(CO). In other embodiments, Y is NMe(CO). In other embodiments, Y is C═O.

In some embodiments,

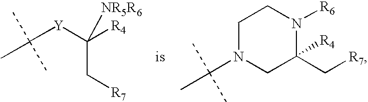

wherein

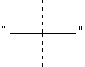

indicate points of attachment. In some embodiments,

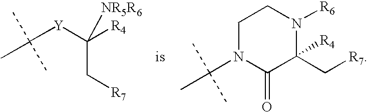

As a note, it is to be understood that when Y is a two or more atom unit that is not symmetric, then Y can be attached to

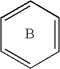

in either orientation; that is, when Y is, for instance CH₂NH, it can be attached to

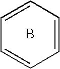

to form

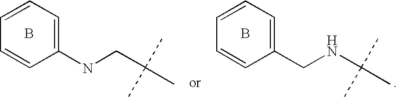

and so on, wherein

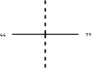

indicates the other point of attachment.

In some embodiments, R₈ₐ is hydrogen.

In some embodiments, R_{8b} is hydrogen.

In some embodiments, compounds of the invention are compounds wherein

R₁ is absent, alkyl, aryl, heteroaryl, aralkoxy, or heteroaralkoxy.

R₄ is hydrogen, alkyl, or alkyl-OH;

R₅ and R₆ are each independently hydrogen or alkyl;

R₇ is selected from the group consisting of —OH, alkyl-OH, —CO₂H, —C(O)O-alkyl, —C(O)O-aryl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, and —C(Z′)(Z″)PO₃Rₚ₁Rₚ₂; wherein

X is CRₓ₁Rₓ₂, NRₓ₃, —O—, —S(O)—, —S(O)₂—, —OS(O)₂—, —OS(O)₂O—, —C(O)—, or —C(O)O—; wherein

Rₓ₃ is hydrogen or alkyl.

In other embodiments, compounds of the invention are compounds wherein

R₁ is absent, alkyl, or aryloxy;

R₄ is hydrogen, alkyl, hydroxy-alkyl, aryl, alkoxy-alkyl, or carboxy-alkyl;

R₅ and R₆ are each independently hydrogen, alkyl, hydroxy-alkyl, aryl, alkoxy-alkyl, —C(O)-alkyl, C(O)-aryl, —C(O)—Oalkyl, or C(O)—Oaryl,;

R₇ is selected from the group consisting of alkyl, —OH, —O-alkyl, —CO₂H, —C(O)O-alkyl, —C(O)O-aryl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, and alkylene-OH, alkylene-CO₂H, alkylene-C(O)O-alkyl, alkylene-C(O)O-aryl, alkylene-CH═CHCO₂H, alkylene-CH₂═CHC(O)O-alkyl, alkylene-CH₂═CHC(O)O-aryl, alkylene-OPO₂Rₚ₁Rₚ₂, -alkylene-OPO₃Rₚ₁Rₚ₂, alkylene-PO₃Rₚ₁Rₚ₂, alkylene-OPO₂(S)Rₚ₁Rₚ₂, any of which may be optionally substituted on carbon with 1, 2, or 3 groups selected from halogen, alkyl, hydroxyl, carboxy, or alkoxy, or any 2 groups on the same carbon may be taken together to from C═O; wherein

Rₚ₁Rₚ₂ are each independently hydrogen, alkyl, or aryl; and

X is CRₓ₁Rₓ₂, NRₓ₃, —O—, —S—, —S(O)—, —SC(O)—, or —C(O)—, wherein Rₓ₁, Rₓ₂, and Rₓ₃ are each independently hydrogen or alkyl.

In other embodiments, compounds of the invention are compounds wherein

R₁ is alkyl, aryl, heteroaryl, aralkoxy, or heteroaralkoxy.

R₄ is hydrogen, alkyl, or alkyl-OH;

R₅ and R₆ are each independently hydrogen or alkyl;

R₇ is selected from the group consisting of —OH, alkyl-OH, —CO₂H, —C(O)O-alkyl, —C(O)O-aryl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, and —C(Z′)(Z″)PO₃Rₚ₁Rₚ₂; wherein

X is CRₓ₁Rₓ₂, NRₓ₃, —O—, —S—, —S(O)—, —S(O)₂—, —OS(O)₂—, —OS(O)₂O—, —C(O)—, or —C(O)O—; wherein

Rₓ₃ is hydrogen or alkyl.

In still other embodiments, compounds of the invention are compounds wherein

R₁ is alkyl, or aryloxy;

R₄ is hydrogen, alkyl, hydroxy-alkyl, aryl, alkoxy-alkyl, or carboxy-alkyl;

R₅ and R₆ are each independently hydrogen, alkyl, hydroxy-alkyl, aryl, alkoxy-alkyl, —C(O)-alkyl, C(O)-aryl, —C(O)—Oalkyl, or C(O)—Oaryl;

R₇ is selected from the group consisting of alkyl, —OH, —O-alkyl, —CO₂H, —C(O)O-alkyl, —C(O)O-aryl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, and alkylene-OH, alkylene-CO₂H, alkylene-C(O)O-alkyl, alkylene-C(O)O-aryl, alkylene-CH═CHCO₂H, alkylene-CH₂═CHC(O)O-alkyl, alkylene-CH₂═CHC(O)O-aryl, alkylene-OPO₂Rₚ₁Rₚ₂, -alkylene-OPO₃Rₚ₁Rₚ₂, alkylene-PO₃Rₚ₁Rₚ₂, alkylene-OPO₂(S)Rₚ₁Rₚ₂, any of which may be optionally substituted on carbon with 1, 2, or 3 groups selected from halogen, alkyl, hydroxyl, carboxy, or alkoxy, or any 2 groups on the same carbon may be taken together to from C═O; wherein

Rₚ₁Rₚ₂are each independently hydrogen, alkyl, or aryl; and

X is CRₓ₁Rₓ₂, NRₓ₃, —O—, —S—, —S(O)—, —S(O)₂—, or —C(O)—, wherein Rₓ₁, Rₓ₂, and Rₓ₃ are each independently hydrogen or alkyl.

In yet other embodiments, compounds of the invention are compounds wherein

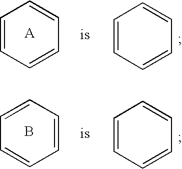

R₁ is aryloxy;

R₄ is hydrogen, alkyl, or alkylene-OH;

R₅ and R₆ are each independently hydrogen or alkyl;

X is CH₂, NH, N-alkyl, —O—, —S—, or —C(O)—; and

Y is

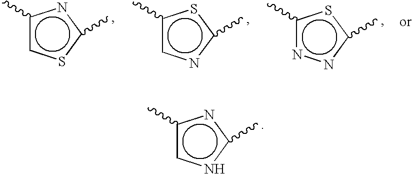

In still other embodiments, compounds of the invention are compounds wherein

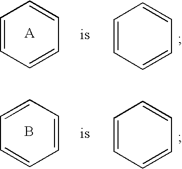

R₁ is aryloxy;

R₄ is hydrogen, alkyl, or alkylene-OH;

R₅ and R₆ are each independently hydrogen or alkyl;.

R₇ is OH, OP(O)₃H₂, or CO₂H;

X is CH₂, NH, N-alkyl, —O—, —S—, or —C(O)—; and

Y is

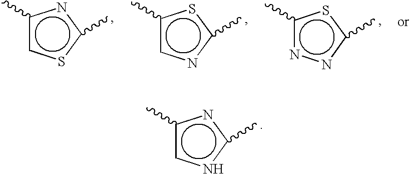

In further embodiments, compounds of the invention are compounds wherein

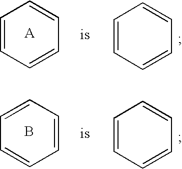

R₁ is aryloxy;

R₃ is CF₃;

a is 0;

b is 1;

R₄ is hydrogen, alkyl, or alkylene-OH;

R₅ and R₆ are each independently hydrogen or alkyl;

R₇ is OH, OP(O)₃H₂, or CO₂H;

X is CH₂, NH, N-alkyl, —O—, —S—, or —C(O)—;

R₈ₐ and R_{8b} are hydrogen; and

Y is

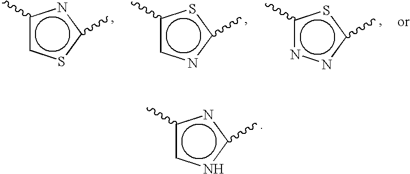

In yet other embodiments, compounds of the invention are compounds wherein

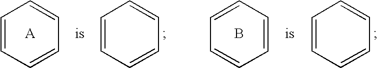

R₂ is aryloxy;

R₄ is hydrogen, alkyl, or alkylene-OH;

R₅ and R₆ are each independently hydrogen or alkyl;

X is CH₂, NH, N-alkyl, —O—, —S—, or —C(O)—; and

Y is CH₂NH, NH(CO), or NMe(CO).

In still other embodiments, compounds of the invention are compounds wherein

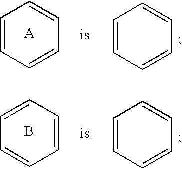

R₂ is aryloxy;

R₄ is hydrogen, alkyl, or alkylene-OH;

R₆ is hydrogen or alkyl;

R₇ is OH, OP(O)₃H₂, or CO₂H;

X is CH₂, NH, N-alkyl, —O—, —S—, or —C(O)—; and

Y is CH₂NH, NH(CO), or NMe(CO).

In other embodiments, compounds of the invention are compounds wherein

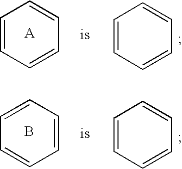

R₂ is aryloxy;

R₃ is CF₃;

a is 0;

b is 1;

R₄ is hydrogen, alkyl, or alkylene-OH;

R₅ and R₆ are each independently hydrogen or alkyl;

R₇ is OH, OP(O)₃H₂, or CO₂H;

X is CH₂, NH, N-alkyl, —O—, —S—, or —C(O)—;

R₈ₐ and R_{8b} are hydrogen; and

Y is CH₂NH, NH(CO), or NMe(CO).

In yet other embodiments, compounds of the invention are compounds wherein

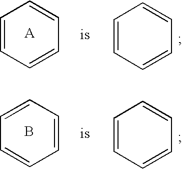

R₂ is aryloxy;

R₄ is hydrogen, alkyl, or alkylene-OH;

R₆ is hydrogen or alkyl;

X is CH₂, NH, N-alkyl, —O—, —S—, or —C(O)—; and

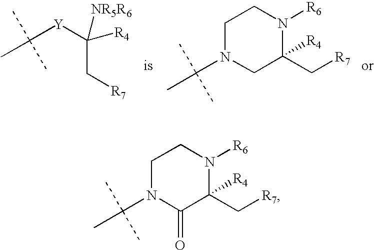

wherein

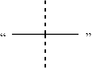

indicate points of attachment.
In yet other embodiments, compounds of the invention are compounds wherein

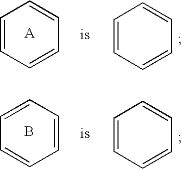

R₂ is aryloxy;

R₄ is hydrogen, alkyl, or alkylene-OH;

R₆ is hydrogen or alkyl;

R₇ is OH, OP(O)₃H₂, or CO₂H;

X is CH₂, NH, N-alkyl, —O—, —S—, or —C(O)—; and

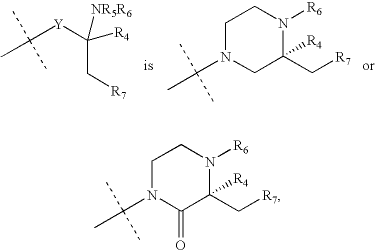

wherein

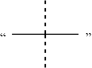

indicate points of attachment.
In further embodiments, compounds of the invention are compounds wherein

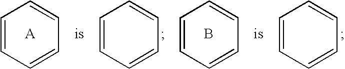

R₂ is aryloxy;

R₃ is CF₃;

a is 0;

b is 1;

R₄ is hydrogen, alkyl, or alkylene-OH;

R₆ is hydrogen or alkyl;

R₇ is OH, OP(O)₃H₂, or CO₂H;

X is CH₂, NH, N-alkyl, —O—, —S—, or —C(O)—;

R₈ₐ and R_{8b} are hydrogen; and

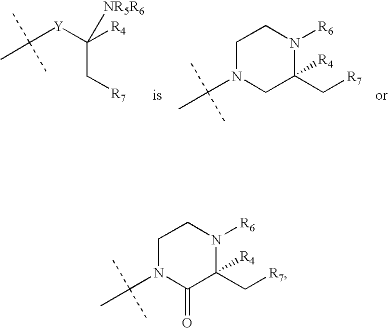

wherein

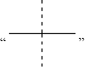

indicate points of attachment.

In further aspects, the present invention is drawn to a compound of formula II.

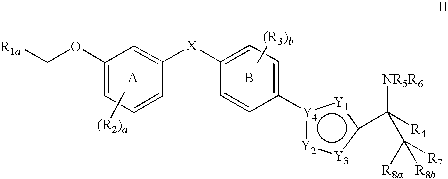

or a pharmaceutically acceptable salt thereof, wherein:

R₁ₐ is aryl or heteroaryl, either of which may be optionally substituted on carbon with 1, 2, or 3 groups selected from halo, alkyl, hydroxyl, or —O-alkyl;

R₂ is halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylSO₂, alkylSO, aralkylSO₂, aralkylSO, alkylene-CO-amino, alkylene-CO-alkylamino, alkylene-CO-dialkylamino, alkylene-NH—CO₂H, alkylene-NH—CO₂alkyl-CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

R₃ is halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl-CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

a and b are each independently 0, 1, 2, or 3;

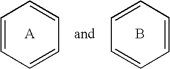

are each independently phenyl or pyridyl;

R₄ is hydrogen, cyano, alkyl, aryl, heteroaryl, alkylene-OH, aryl, alkylene-O-alkyl, —CO₂H, —CO₂-alkyl, alkylene-CO₂H, or alkylene-CO₂-alkyl, alkylene-OC(O)R wherein R is hydrogen or alkyl; cycloalkyl, heterocycloalkyl, alkylene-NH₂, alkylene-alkylamino, or alkylene-dialkylamino, any of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

R₅ and R₆ are each independently selected from the group consisting of hydrogen, alkyl, alkylene-OH, aryl, alkylene-O-alkyl, —CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)—Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or

R₅ and R₆, together with the nitrogen to which they are attached, may form a 3, 4, 5, or 6-membered saturated or unsaturated ring, optionally containing 1 or 2 additional heteroatoms selected from O, S, NH, or N-alkyl, and optionally substituted on carbon with halogen, alkyl, hydroxyl, or alkoxy;

R₇ is selected from the group consisting of alkyl, —OH, —O-alkyl, —CO₂H, —C(O)O-alkyl, —C(O)O-aryl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, —OPO₂(S)NRₚ₁Rₚ₂, and —C(Z′)(Z″)PO₃Rₚ₁Rₚ₂, alkylene-OH, alkylene-CO₂H, alkylene-C(O)O-alkyl, alkylene-C(O)O-aryl, alkylene-CH═CHCO₂H, alkylene-CH₂═CHC(O)O-alkyl, alkylene-CH₂═CHC(O)O-aryl, alkylene-OPO₂Rₚ₁Rₚ₂, -alkylene-OPO₃Rₚ₁Rₚ₂, alkylene-PO₃Rₚ₁Rₚ₂, alkylene-OPO₂(S)Rₚ₁Rₚ₂, and alkylene-C(Z′)(Z″)PO₃Rₚ₁Rₚ₂, any of which may be optionally substituted on carbon with 1, 2, or 3 groups selected from halogen, alkyl, hydroxyl, carboxy, or alkoxy, or any 2 groups on the same carbon may be taken together to from C═O; and wherein

Z′ is hydroxyl or halogen;

Z″ is H or halogen;

Rₚ₁ and Rₚ₂ at each occurrence are independently hydrogen, C₁-C₆-alkyl, or aryl;

X is CRₓ₁Rₓ₂, NRₓ₃, —(CRₓ₁Rₓ₂)ₙNRₓ₃—, —NRₓ₃(CRₓ₁Rₓ₂)ₙ—, O, —S—, —(CRₓ₁Rₓ₂)ₙS—, —S(CRₓ₁Rₓ₂)ₙ—, —S(O)—, —(CRₓ₁Rₓ₂)ₙS(O)—, —S(O)(CRₓ₁Rₓ₂)ₙ—, —S(O)₂—, —(CRₓ₁Rₓ₂)ₙS(O)₂—, —S(O)₂(CRₓ₁Rₓ₂)ₙ—, —C(O)—, —(CRₓ₁Rₓ₂)ₙC(O)—, —C(O)(CRₓ₁Rₓ₂)ₙ—, —C(O)O—, —(CRₓ₁Rₓ₂)ₙC(O)O—, and —C(O)O(CRₓ₁Rₓ₂)ₙ—; wherein

Rₓ₁ and Rₓ₂ at each occurrence are independently selected from the group consisting of hydrogen, hydroxyl, halogen, alkyl, —O-alkyl, alkylyene-O-alkyl, alkyl-SO₂, CO₂H, and CO₂-alkyl, any of which may be optionally substituted on carbon with halogen; or taken together Rₓ₁ and Rₓ₂ may form a 3, 4, 5, or 6 membered ring optionally containing 1 or 2 heteroatoms selected from O, S, NH, or N-alkyl, which may itself be substituted on carbon with halogen, hydroxyl, or alkyl;

Rₓ₃ at each occurrence is selected from the group consisting of hydrogen and alkyl;

n is an integer from 0 to 4; and

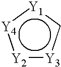

is a heteroaryl ring containing up to four heteroatoms selected from N, O, or S, optionally substituted on carbon with halogen or alkyl, wherein

Y₁ is N, S, or O;

Y₂ and Y₃ are each independently C, N, O, or S; provided that when

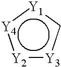

contains an N—H, that hydrogen may be replaced with alkyl;

Y₄ is C or N; and

R₈ₐ and R_{8b} are each independently hydrogen, halogen, alkyl, or taken together with the carbon to which they are attached, may form a 3, 4, 5, or 6-membered ring, optionally containing 1 or 2 heteroatoms selected from NH, N-alkyl, O, or S, and optionally substituted on carbon with halogen, or alkyl.

It is to be understood that when the values for a and b are less than the total number of open substituents on the ring, the remainder of the substituents are hydrogen.

That is, if b is 1, the remaining three substituents on

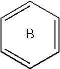

are hydrogen.

In some embodiments, R₁ₐ is phenyl.

In some embodiments, a is 0. In other embodiments, a is 1.

In some embodiments, b is 1.

In some embodiments,

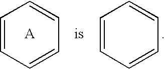

In other embodiments,

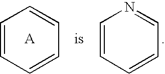

In some embodiments,

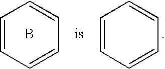

In other embodiments,

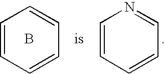

As provided above, the compounds of formula II include an SEM. The SEM may be a halogen such as F or Cl. It may also be a halo-substituted alkyl group such as CF₃, CF₂CF₃, CF₂CF₂CF₃, CFHCF₃, CH₂CF₃, CH₂CH₂CF₃, CHCl₂, or CH₂Cl. It may also be cyano.

Thus, in some embodiments, R₃ is trifluoromethyl. In some embodiments, R₃ is methyl. In other embodiments, R₃ is dimethylamino. In other embodiments, R₃ is fluoro. In other embodiments, R₃ is chloro. In other embodiments, R₃ is bromo. In other embodiments, R₃ is cyano. In other embodiments, R₃ is dimethylamino.

In some embodiments, R₄ is hydrogen. In other embodiments, R₄ is methyl. In other embodiments, R₄ is hydroxymethyl.

In some embodiments, R₅ and R₆ are independently hydrogen.

In some embodiments, R₇ is OH. In other embodiments, R₇ is CO₂H. In other embodiments, R₇ is CO₂Me or CO₂Et. Another In other embodiments, R₇ is CO₂-phenyl. In other embodiments, R₇ is —OP(O)₃H₂.

In some embodiments, X is CH₂. In other embodiments, X is NH or N-alkyl. In other embodiments, X is O. In other embodiments, X is S, SO, or SO₂. In other embodiments, X is CO.

In some embodiments, Y is

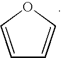

In other embodiments, Y is

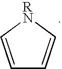

In other embodiments, Y is

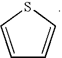

In other embodiments, Y is

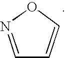

In other embodiments, Y is

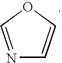

In some embodiments, Y is

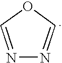

In other embodiments, Y is

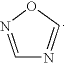

In other embodiments, Y is

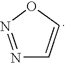

In other embodiments, Y is

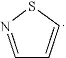

In some embodiments, Y is

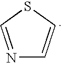

In other embodiments, Y is

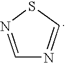

In other embodiments, Y is

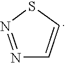

In other embodiments, Y is

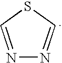

Another specific value for Y is

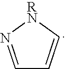

In some embodiments,

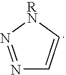

In other embodiments, Y is

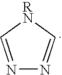

In other embodiments, Y is

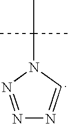

In other embodiments, Y is

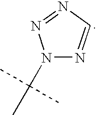

In the above structures, R can be hydrogen or alkyl, and

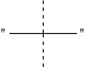

indicates a point of attachment.

In some embodiments, R₈ₐ is hydrogen.

In some embodiments, R_{8b} is hydrogen.

In some embodiments, compounds of the invention are compounds wherein

R₁ₐ is phenyl;

R₄ is hydrogen, alkyl, or alkyl-OH;

R₅ and R₆ are each independently hydrogen or alkyl;

R₇ is selected from the group consisting of —OH, alkyl-OH, —CO₂H, —C(O)O-alkyl, —C(O)O-aryl, —CH₂═CHCOO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, and —C(Z′)(Z″)PO₃Rₚ₁Rₚ₂; wherein

X is CRₓ₁Rₓ₂NRₓ₃, O, —S—, —S(O)—, —S(O)₂—, —OS(O)₂—, —OS(O)₂O—, —C(O)—, or —C(O)O—; wherein

Rₓ₃ is hydrogen or alkyl;

In other embodiments, compounds of the invention are compounds wherein

R₁ₐ is phenyl;

R₄ is hydrogen, alkyl, hydroxy-alkyl, aryl, alkoxy-alkyl, or carboxy-alkyl;

R₅ and R₆ are each independently hydrogen, alkyl, hydroxy-alkyl, aryl, alkoxy-alkyl, —C(O)-alkyl, C(O)-aryl, —C(O)—Oalkyl, or C(O)—Oaryl;

R₇ is selected from the group consisting of alkyl, —OH, —O-alkyl, —CO₂H, —C(O)O-alkyl, —C(O)O-aryl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, and alkylene-OH, alkylene-CO₂H, alkylene-C(O)O-alkyl, alkylene-C(O)O-aryl, alkylene-CH═CHCO₂H, alkylene-CH₂═CHC(O)O-alkyl, alkylene-CH₂═CHC(O)O-aryl, alkylene-OPO₂Rₚ₁Rₚ₂, -alkylene-OPO₃Rₚ₁Rₚ₂, alkylene-PO₃Rₚ₁Rₚ₂, alkylene-OPO₂(S)Rₚ₁Rₚ₂, any of which may be optionally substituted on carbon with 1, 2, or 3 groups selected from halogen, alkyl, hydroxyl, carboxy, or alkoxy, or any 2 groups oh the same carbon may be taken together to from C═O; wherein

Rₚ₁Rₚ₂ are each independently hydrogen, alkyl, or aryl; and

X is CRₓ₁Rₓ₂, NRₓ₃, O, —S—, —S(O)—, —S(O)₂—, or —C(O)—, wherein Rₓ₁, Rₓ₂, and

Rₓ₃ are each independently hydrogen or alkyl.
In other embodiments, compounds of the invention are compounds wherein

R₁ₐ is phenyl;

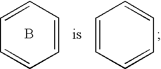

R₁ is aryloxy;

R₄ is hydrogen, alkyl, or alkylene-OH;

R₅ and R₆ are each independently hydrogen or alkyl; and

R₈ₐ and R_{8b} are hydrogen;

In yet other embodiments, compounds of formula II are compounds of formula II-1.

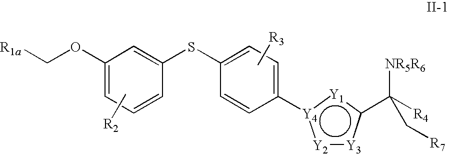

and pharmaceutically acceptable salts thereof.

In other embodiments, compounds of formula II are compounds of formula II-2A or II-2B.

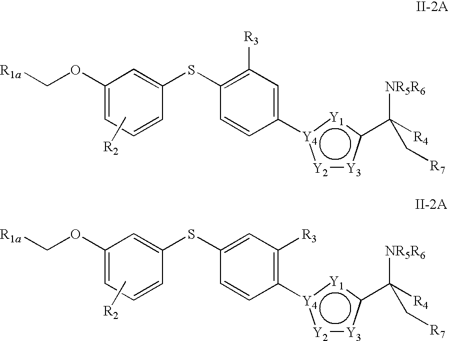

and pharmaceutically acceptable salts thereof.

In further embodiments, compounds of formula II are compounds of formula II-3A or II-3B.

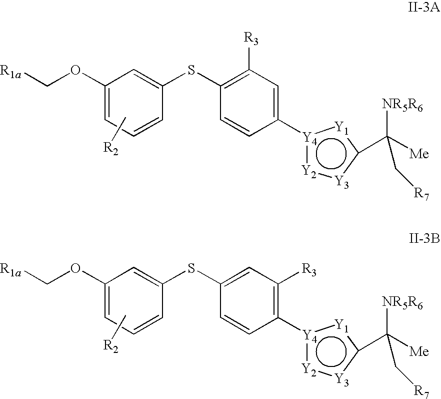

and pharmaceutically acceptable salts thereof.

In other embodiments, compounds of formula II are compounds of formula II-4A or II-4B.

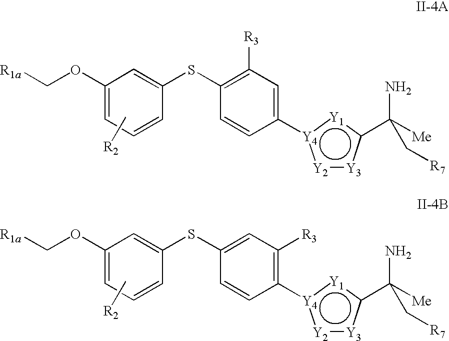

and pharmaceutically acceptable salts thereof.

In still other embodiments, compounds of formula II are compounds of formula II-5A or II-5B.

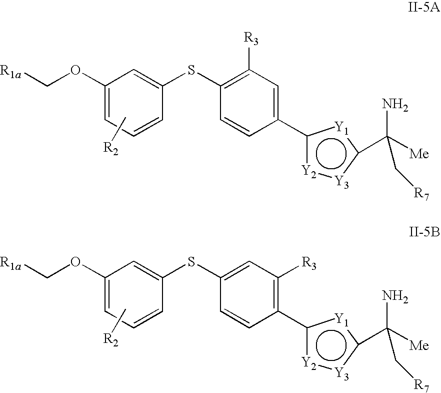

and pharmaceutically acceptable salts thereof.

In other embodiments, compounds of formula II are compounds of formula II-6A or II-6B.

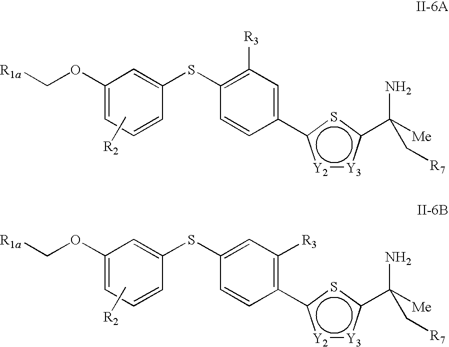

and pharmaceutically acceptable salts thereof.

In further embodiments, compounds of formula II are compounds of formula II-7A or II-7B.

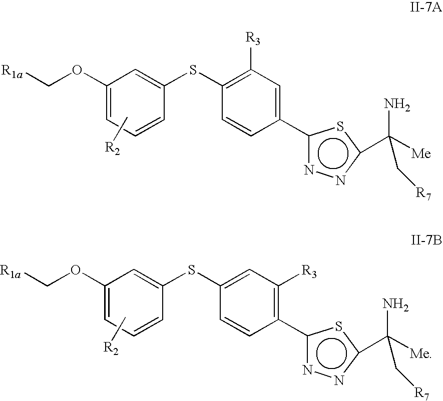

and pharmaceutically acceptable salts thereof.

In other embodiments, compounds of formula II are compounds of formula II-8A or II-8B.

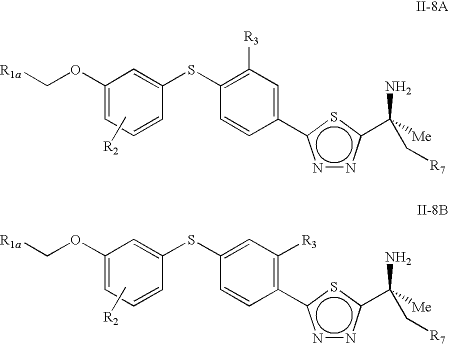

and pharmaceutically acceptable salts thereof.

In still other aspects, the present invention is drawn to a compound of formula III.

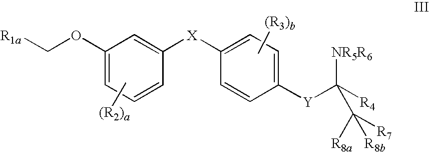

or a pharmaceutically acceptable salt thereof, wherein:

R₁ₐ is aryl or heteroaryl, either of which may be optionally substituted on carbon with 1, 2, or 3 groups selected from halo, alkyl, hydroxyl, or —O-alkyl;

R₂ is halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylSO₂, alkylSO, aralkylSO₂, aralkylSO, alkylene-CO-amino, alkylene-CO-alkylamino, alkylene-CO-dialkylamino, alkylene-NH—CO₂H, alkylene-NH—CO₂alkyl-CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

R₃ is halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl-CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

a is 0, 1, 2, or 3;

b is 1, 2, or 3;

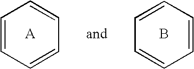

are each independently phenyl or pyridyl;

R₄ is hydrogen, cyano, alkyl, aryl, heteroaryl, alkylene-OH, aryl, alkylene-O-alkyl, —CO₂H, —CO₂-alkyl, alkylene-CO₂H, or alkylene-CO₂-alkyl, alkylene-OC(O)R wherein R is hydrogen or alkyl; cycloalkyl, heterocycloalkyl, alkylene-NH₂, alkylene-alkylamino, or alkylene-dialkylamino, any of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

R₅ and R₆ are each independently selected from the group consisting of hydrogen, alkyl, alkylene-OH, aryl, alkylene-O-alkyl, —CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)—Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or

R₅ and R₆, together with the nitrogen to which they are attached, may form a 3, 4, 5, on 6-membered saturated or unsaturated ring, optionally containing 1 or 2 additional heteroatoms selected from O, S, NH, or N-alkyl, and optionally substituted on carbon with halogen, alkyl, hydroxyl, or alkoxy;

R₇ is selected from the group consisting of alkyl, —OH, —O-alkyl, —CO₂H, —C(O)O-alkyl, —C(O)O-aryl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, —OPO₂(S)NRₚ₁Rₚ₂, and —C(Z′)(Z″)PO₃Rₚ₁Rₚ₂, alkylene-OH, alkylene-CO₂H, alkylene-C(O)O-alkyl, alkylene-C(O)O-aryl, alkylene-CH═CHCO₂H, alkylene-CH₂═CHC(O)O-alkyl, alkylene-CH₂═CHC(O)O-aryl, alkylene-OPO₂Rₚ₁Rₚ₂, -alkylene-OPO₃Rₚ₁Rₚ₂, alkylene-PO₃Rₚ₁Rₚ₂, alkylene-OPO₂(S)Rₚ₁Rₚ₂, and alkylene-C(Z′)(Z″)PO₃Rₚ₁Rₚ₂, any of which may be optionally substituted on carbon with 1, 2, or 3 groups selected from halogen, alkyl, hydroxyl, carboxy, or alkoxy, or any 2 groups on the same carbon may be taken together to from C═O; and wherein

Z′ is hydroxyl or halogen;

Z″ is H or halogen;

Rₚ₁ and Rₚ₂ at each occurrence are independently hydrogen, C₁-C₆-alkyl, or aryl;

X is CRₓ₁Rₓ₂, NRₓ₃, —(CRₓ₁Rₓ₂)ₙNRₓ₃, —NRₓ₃(CRₓ₁Rₓ₂)ₙ—, O, —S—, —(CRₓ₁Rₓ₂)ₙS—, —S(CRₓ₁Rₓ₂)ₙ—, —S(O)—, —(CRₓ₁Rₓ₂)ₙS(O)—, —S(O)(CRₓ₁Rₓ₂)ₙ—, —S(O)₂—, —(CRₓ₁Rₓ₂)ₙS(O)₂—, —S(O)₂(CRₓ₁Rₓ₂)ₙ—, —C(O)—, —(CRₓ₁Rₓ₂)ₙC(O)—, —C(O)(CRₓ₁Rₓ₂)ₙ—, —C(O)O—, —(CRₓ₁Rₓ₂)ₙC(O)O—, and —C(O)O(CRₓ₁Rₓ₂)ₙ—; wherein

Rₓ₁ and Rₓ₂ at each occurrence are independently selected from the group consisting of hydrogen, hydroxyl, halogen, alkyl, —O-alkyl, alkylyene-O-alkyl, alkyl-SO₂, CO₂H, and CO₂-alkyl, any of which may be optionally substituted on carbon with halogen; or taken together Rₓ₁ and Rₓ₂ may form a 3, 4, 5, or 6 membered ring optionally containing 1 or 2 heteroatoms selected from O, S, NH, or N-alkyl, which may itself be substituted on carbon with halogen, hydroxyl, or alkyl;

Rₓ₃ at each occurrence is selected from the group consisting of hydrogen and alkyl;

n is an integer from 0 to 4;

Y is —CH₂NR′″—, —CH₂NR′″(CO)—, —CHFNR′″—, —CHFNR′″(CO)—, —CF₂NR′″—, —CF₂NR′″(CO)—, —CH₂(CO)—, —CHF(CO)—, —CF₂(CO)—, —(CO)CF₂—, —CH₂(CHOH)—, —CHF(CHOH)—, —CF₂(CHOH)—, —(CHOH)CF₂—, —NH(CO)—, —(CO)—, —(CO)₂—, —O—, —S—, —SO—, —SO₂—, —CH₂O—, —CH₂CH₂O—, —CH₂OCH₂—, —OCH₂O—, —CH₂S—, —CH₂SO—, —CH₂SO₂—, —CHFO—, —CHFS—, —CHFSO—, —CHFSO₂—, —CF₂O—, —CF₂S—, —CF₂SO—, —CF₂SO₂—, —NR′″SO₂—, —CF₂—, —CF₂CF₂—, —CF₂CF₂CF₂—, wherein R′″ is H or alkyl; and

R₈ₐ and R_{8b} are each independently hydrogen, halogen, alkyl, or taken together with the carbon to which they are attached, may form a 3, 4, 5, or 6-membered ring, optionally containing 1 or 2 heteroatoms selected from NH, N-alkyl, O, or S, and optionally substituted on carbon with halogen, or alkyl.

It is to be understood that when the values for a and b are less than the total number of open substituents on the ring, the remainder of the substituents are hydrogen.

That is, if b is 1, the remaining three substituents on

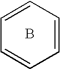

are hydrogen.

In some embodiments, R₁ₐ is phenyl.

In some embodiments, a is 0. In other embodiments, a is 1.

In some embodiments, b is 1.

In some embodiments,

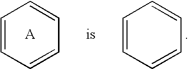

In other embodiments,

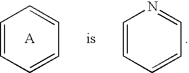

In some embodiments,

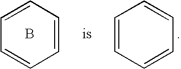

In other embodiments,

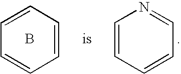

As provided above, the compounds of formula III include an SEM. The SEM may be a halogen such as F or Cl. It may also be a halo-substituted alkyl group such as CF₃, CF₂CF₃, CF₂CF₂CF₃, CFHCF₃, CH₂CF₃, CH₂CH₂CF₃, CHCl₂, or CH₂Cl. It may also be cyano.

Thus, in some embodiments, R₃ is trifluoromethyl. In some embodiments, R₃ is methyl. In other embodiments, R₃ is dimethylamino. In other embodiments, R₃ is fluoro. In other embodiments, R₃ is chloro. In other embodiments, R₃ is bromo. In other embodiments, R₃ is cyano. In other embodiments, R₃ is dimethylamino.

In some embodiments, R₄ is hydrogen. In other embodiments, R₄ is methyl. In other embodiments, R₄ is hydroxymethyl.

In some embodiments, R₅ and R₆ are independently hydrogen.

In some embodiments, R₇ is OH. In other embodiments, R₇ is CO₂H. In other embodiments, R₇ is CO₂Me or CO₂Et. Another In other embodiments, R₇ is CO₂-phenyl. In other embodiments, R₇ is —OP(O)₃H₂.

In some embodiments, X is CH₂. In other embodiments, X is NH or N-alkyl. In other embodiments, X is O. In other embodiments, X is S, SO, or SO₂. In other embodiments, X is CO.

In some embodiments, Y is CH₂. In other embodiments, Y is CH₂NH. In other embodiments, Y is NH(CO). In other embodiments, Y is NMe(CO). In other embodiments, Y is C═O.

In some embodiments,

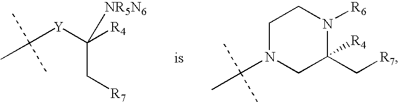

wherein

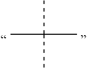

indicate points of attachment. In some embodiments,

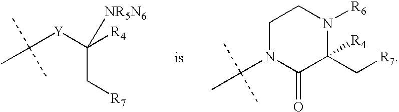

In some embodiments, R₈ₐ is hydrogen.

In some embodiments, R_{8b} is hydrogen.

In some embodiments, compounds of the invention are compounds wherein

R₂ is alkyl, aryl, heteroaryl, aralkoxy, or heteroaralkoxy.

R₄ is hydrogen, alkyl, or alkyl-OH;

R₅ and R₆ are each independently hydrogen or alkyl;

R₇ is selected from the group consisting of —OH, alkyl-OH, —CO₂H, —C(O)O-alkyl, —C(O)O-aryl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, and —C(Z′)(Z″)PO₃Rₚ₁Rₚ₂; wherein

X is CRₓ₁Rₓ₂, NRₓ₃, O, —S—, —S(O)—, —S(O)₂—, —OS(O)₂—, —OS(O)₂O—, —C(O)—, or —C(O)O—; wherein

Rₓ₃ is hydrogen or alkyl;

In other embodiments, compounds of the invention are compounds wherein

R₂ is hydrogen, alkyl, or aryloxy;

R₄ is hydrogen, alkyl, hydroxy-alkyl, aryl, alkoxy-alkyl, or carboxy-alkyl;

R₅ and R₆ are each independently hydrogen, alkyl, hydroxy-alkyl, aryl, alkoxy-alkyl, —C(O)-alkyl, C(O)-aryl, —C(O)—Oalkyl, or C(O)—Oaryl;

R₇ is selected from the group consisting of alkyl, —OH, —O-alkyl, —CO₂H, —C(O)O-alkyl, —C(O)O-aryl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, and alkylene-OH, alkylene-CO₂H, alkylene-C(O)O-alkyl, alkylene-C(O)O-aryl, alkylene-CH═CHCO₂H, alkylene-CH₂═CHC(O)O-alkyl, alkylene-CH₂═CHC(O)O-aryl, alkylene-OPO₂Rₚ₁Rₚ₂, -alkylene-OPO₃Rₚ₁Rₚ₂, alkylene-PO₃Rₚ₁Rₚ₂, alkylene-OPO₂(S)Rₚ₁Rₚ₂, any of which may be optionally substituted on carbon with 1, 2, or 3 groups selected from halogen, alkyl, hydroxyl, carboxy, or alkoxy, or any 2 groups on the same carbon may be taken together to from C═O; wherein

Rₚ₁Rₚ₂ are each independently hydrogen, alkyl, or aryl; and

X is CRₓ₁Rₓ₂, NRₓ₃, O, —S—, —S(O)—, —S(O)₂—, or —C(O)—, wherein Rₓ₁, Rₓ₂, and Rₓ₃ are each independently hydrogen or alkyl.

In still other embodiments, compounds of the invention are compounds wherein

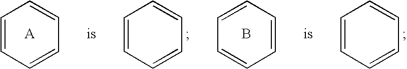

R₁ₐ is aryl;

R₄ is hydrogen, alkyl, or alkylene-OH;

R₅ and R₆ are each independently hydrogen or alkyl; and

R₈ₐ and R_{8 b} are hydrogen;

In further embodiments, compounds of formula III are compounds of formula III-1.

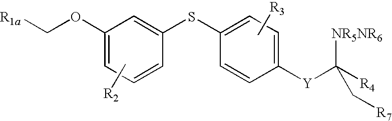

and pharmaceutically acceptable salts thereof.

In other embodiments, compounds of formula III are compounds of formula III-2A or III-2B.

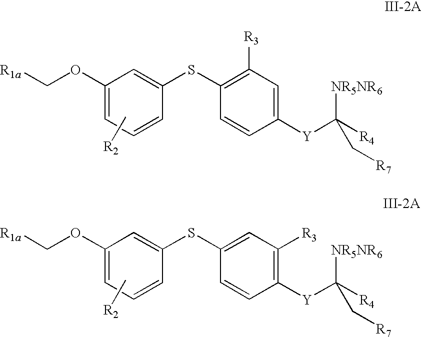

and pharmaceutically acceptable salts thereof.

In some embodiments, compounds of formula III are compounds of formula III-3A, III-3B, III-3C, or III-3D.

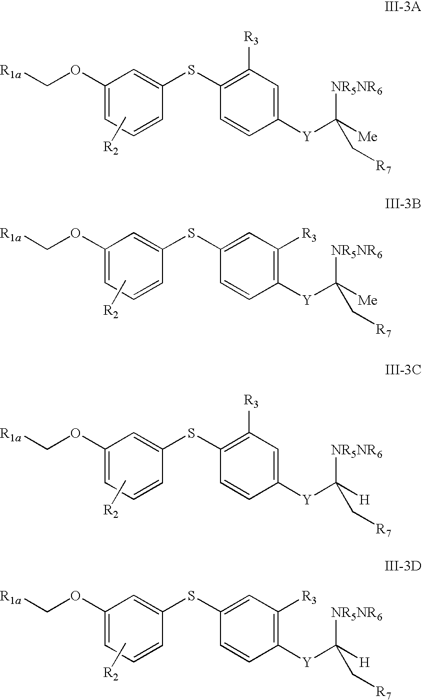

and pharmaceutically acceptable salts thereof.

In other embodiments, compounds of formula III are compounds of formula III-4A, III-4B, III-4C, or III-4D.

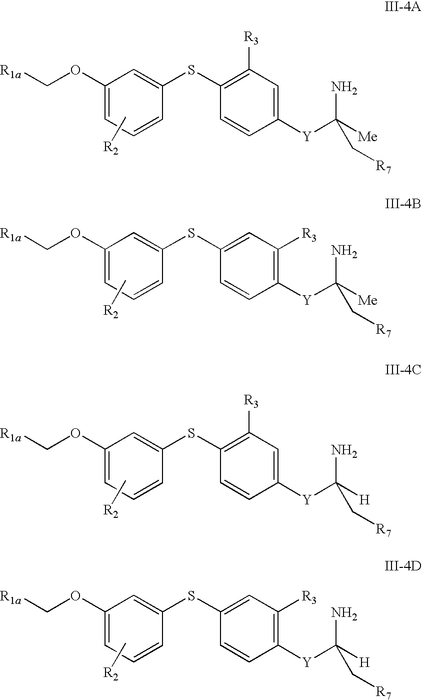

and pharmaceutically acceptable salts thereof.

In some embodiments, compounds of the present invention include compounds listed in the following table:

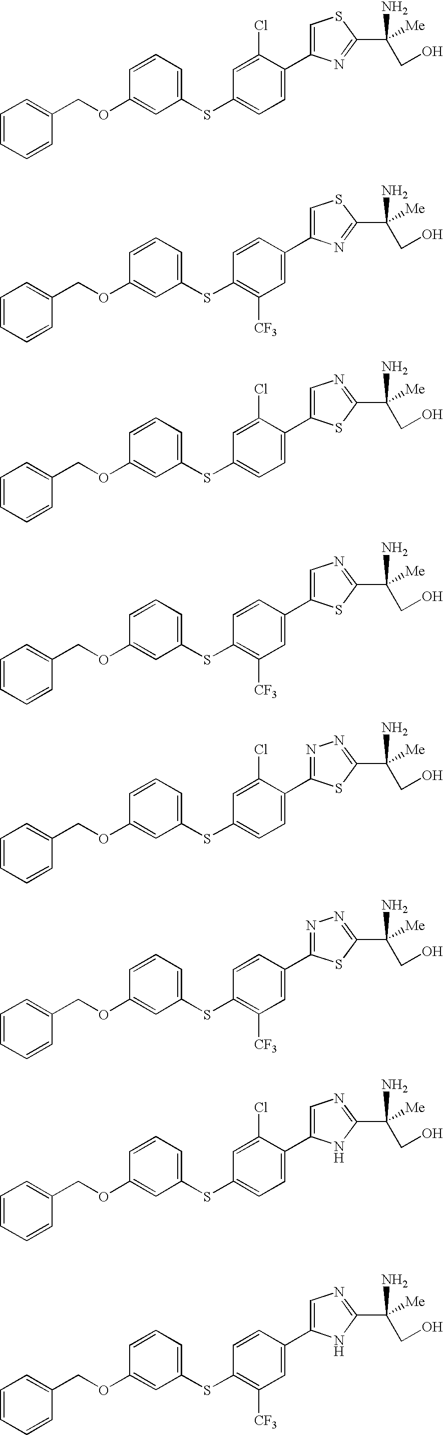

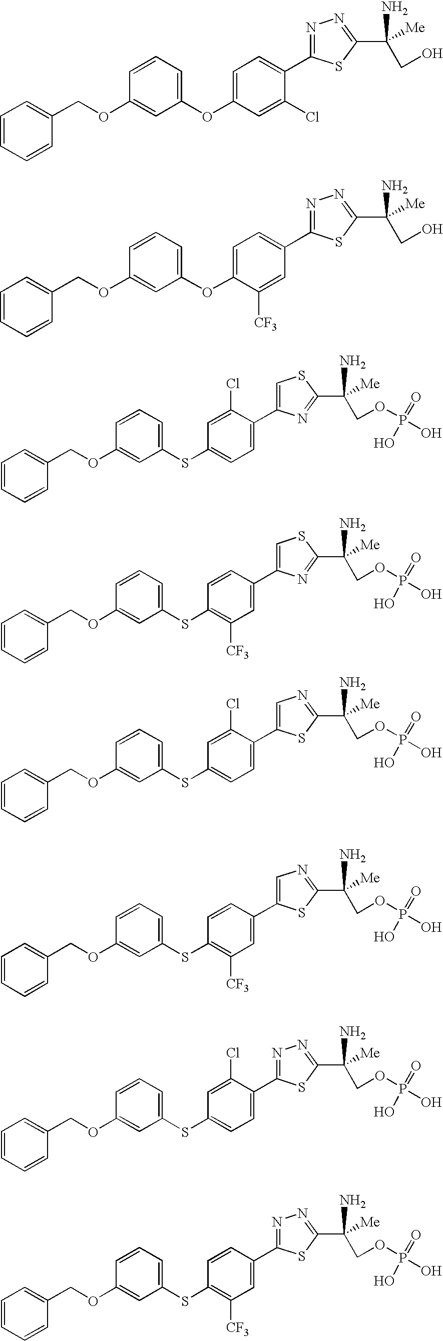

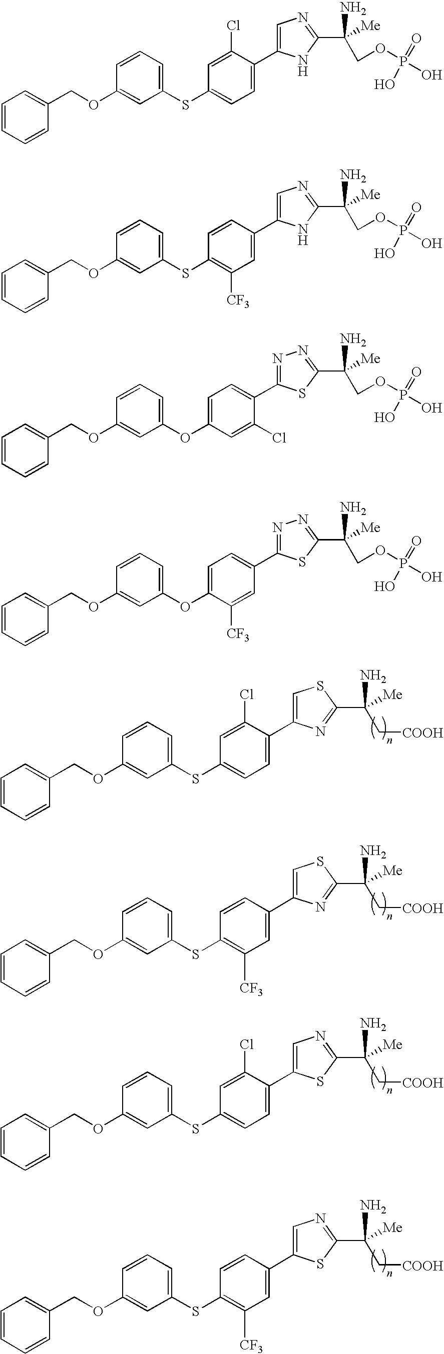

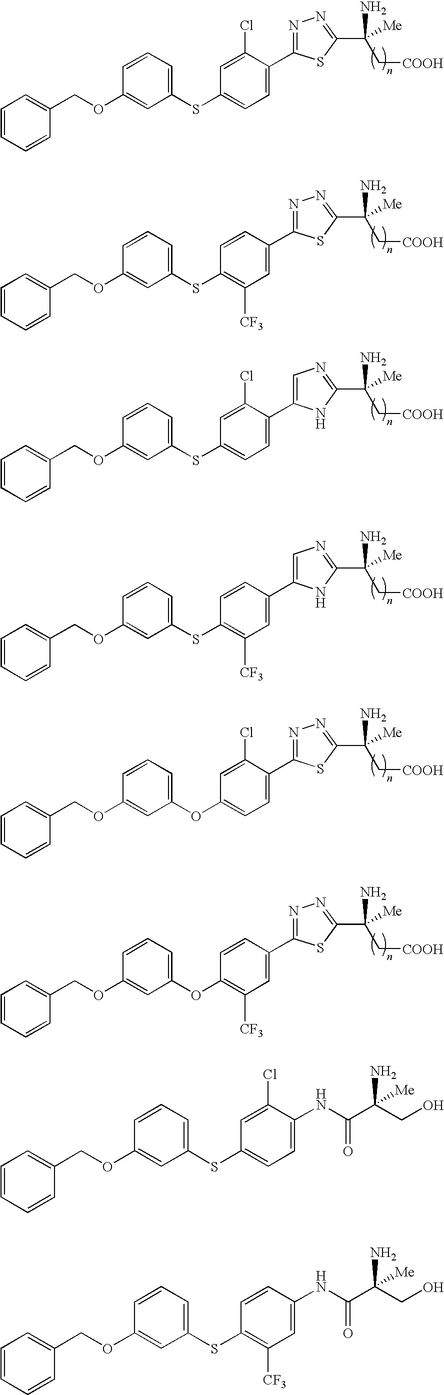

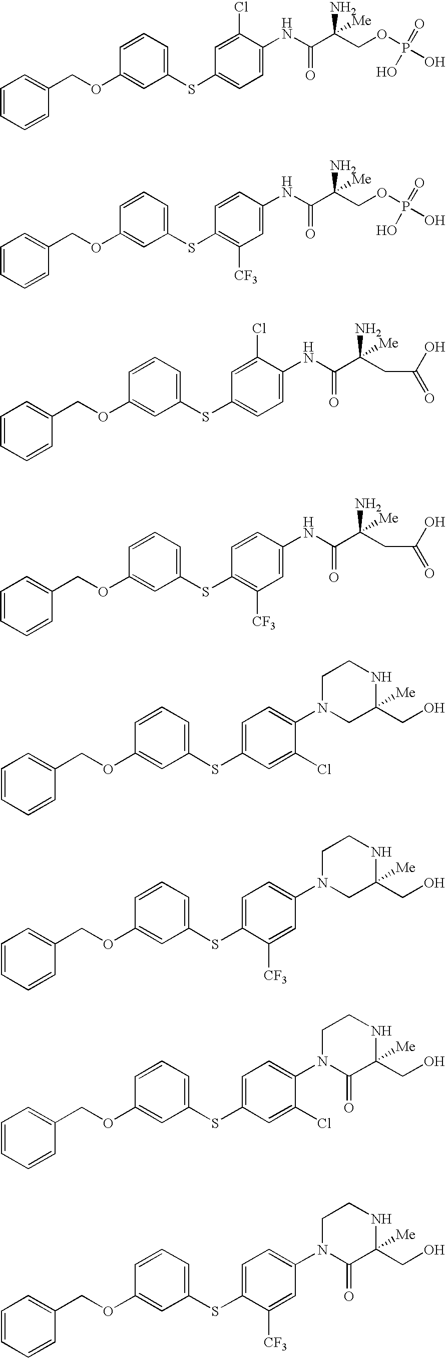

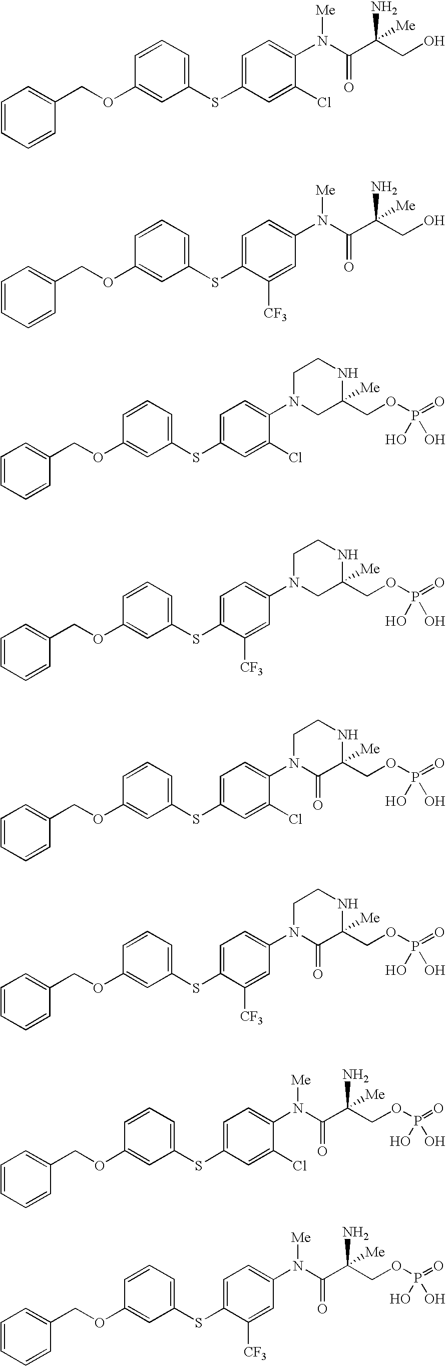

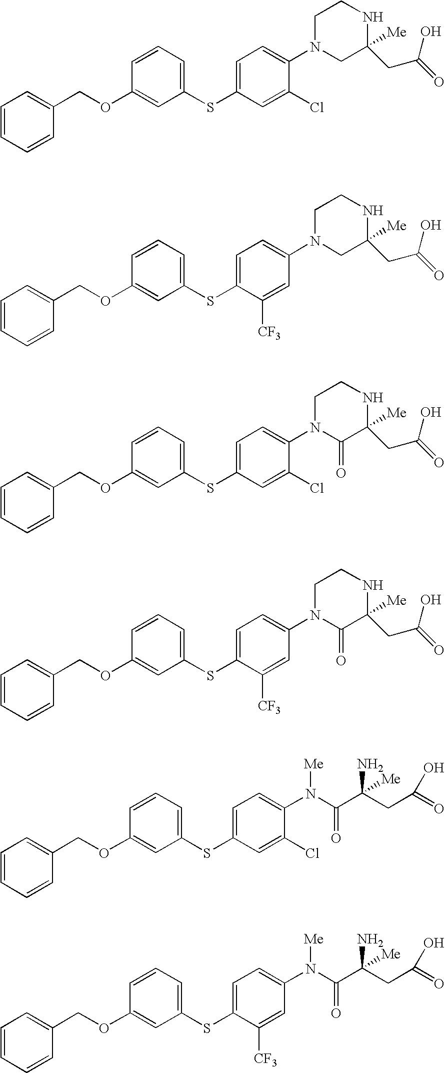

wherein n is 0, 1, or 2 for the above compounds, as well as pharmaceutically acceptable salts, phosphate derivatives, phosphate mimics, or phosphate precursor analogs thereof.

In some embodiments of the present invention, R₂ is not aryl. In other embodiments, of the present invention, X is not —O—. In still other embodiments of the invention, the compound is not a compound as described in WO 06/020951, published Feb. 23, 2006. In still other embodiments, the compound is not a compound as described in U.S. Publication No. 20060223866, published Oct. 5, 2006.

### Preparation of Invention Compounds

The general approaches for the synthesis of invention compounds are summarized in Scheme 1 and 2. Reaction of substituted 3-mercaptophenol with substituted 4-fluoroacetophenone 1 and benzyl bromide afforded the thio-ether-acetophenone intermediate 2. The thio-ether-acetophenone 2 was then converted to the α-bromo-acetophenone 3 by reaction with tetrabutylammonium tribromide. Reaction of the bromo-acetophenone with Boc-protected α-amino acids followed by cyclizatioin with ammonium acetate afforded the imidazole analog 4. Removal of the Boc group provided the TFA salt of the final compound 5 in good yield.

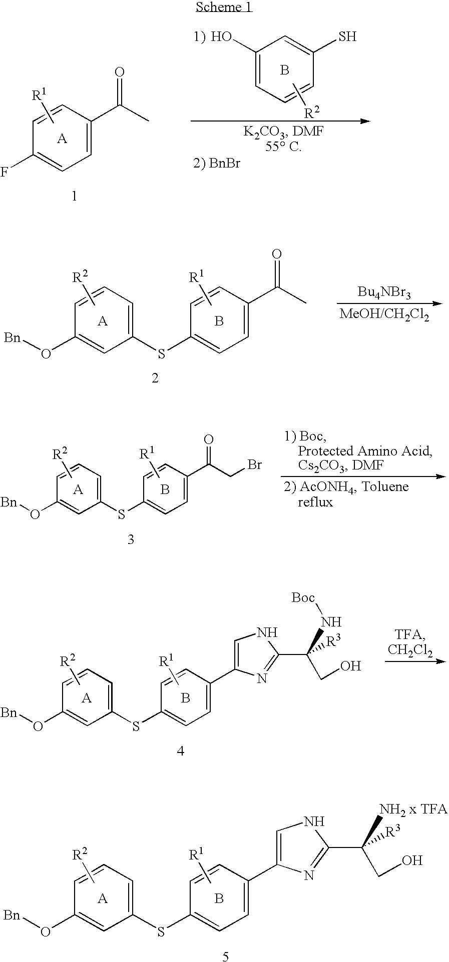

Reaction of substituted benzoate 6 with 3-mercaptophenol followed by benzyl bromide afforded the thio-either 7 which upon refluxing with hydrazine in ethylene glycol provided hydrazide 8. Hydrazide 8 was then coupled with orthogonally protected α-methylserine 9 and cyclized to thiadiazole 11 by reaction with Lawesson's reagent. Thiadiazole 11 deprotection afforded the final alcohol 12 in reasonable overall yield.

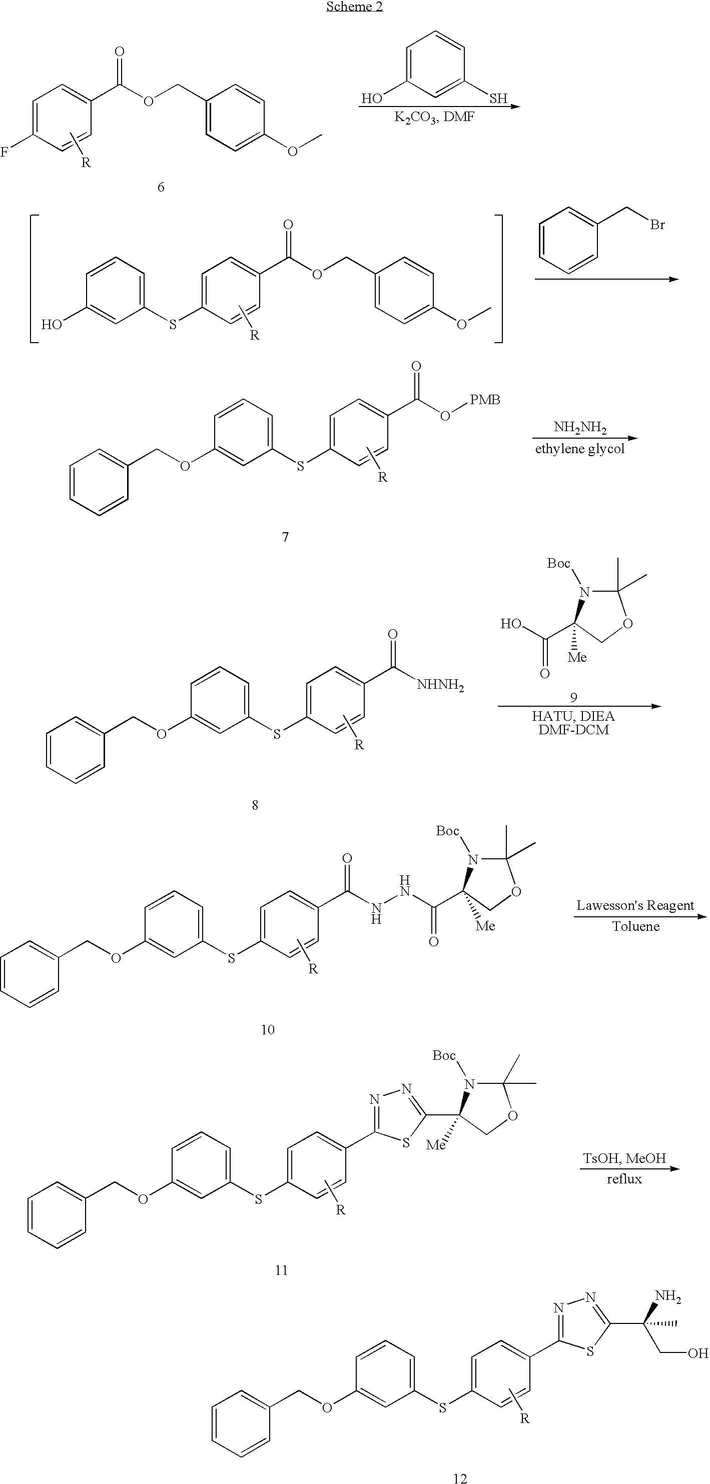

### Biological Activity of Invention Compounds

Lymphopenia Assay

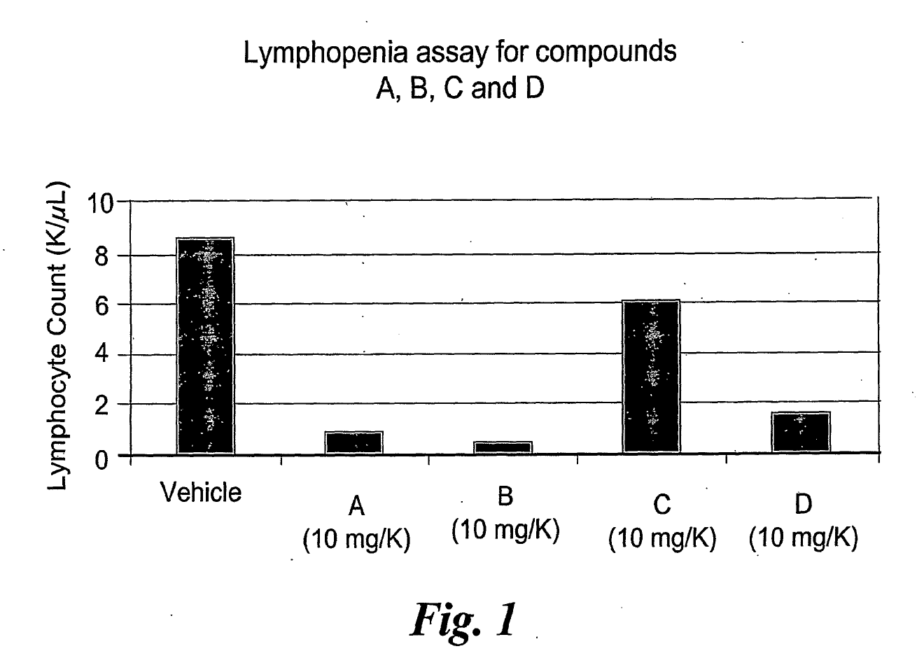

Several compounds were evaluated for the ability to induce lymphopenia in mice. Male C57B1/6 mice were divided into three groups. The control group received the 3% BSA vehicle only. The other two groups received a single dose of test compound in vehicle by oral administration (PO) and intravenous administration (IV), respectively. After 6 hours, the mice were anesthesized with isoflurane and approximately 250 μL of blood was removed from the retroorbital sinus and collected in an EDTA microtainer, mixed with an anticoagulant and placed on a tilt table until complete blood count (CBC) analysis. Table 1 and FIG. 1 illustrate the results of this lymphopenia assay performed with S1P-1 agonists. It can be seen from these results that oral administration (10 mg/kg) of these compounds induced significant lymphopenia compared to the control.

TABLE 1
Structures of Compounds A through D, tested as S1P1 receptor agonists.

| Structure | Identification |
|---|---|
| 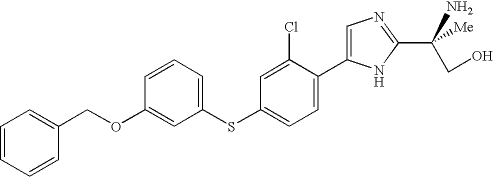 | A |
| 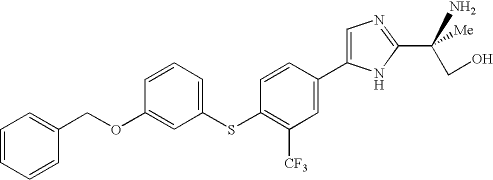 | B |
| 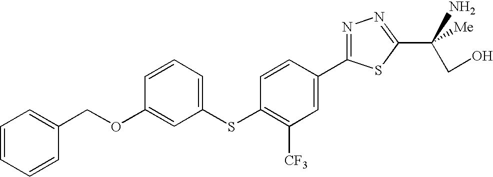 | C |
| 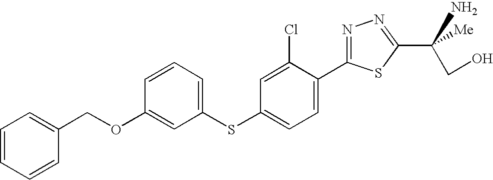 | D |

Interaction of Invention Compounds with SIP Receptors

In certain embodiments, the compounds of the invention selective for the S1P-1 receptor as compared to one or more of the other S1P receptors. For example, one set of compounds includes compounds which are selective for the S1P-1 receptor relative to the S1P-3 receptor.

A compound is “selective” for the S1P-1 receptor relative to a second receptor, if the IC₅₀ of the compound for the second receptor is at least two-fold greater than the IC₅₀ for the S1P-1 receptor. The IC₅₀ of a compound is determined using the [³³P]sphingosine 1-phosphate binding assay, as described in Davis, M. D. et al., Sphingosine 1-Phosphate Analogs as Receptor Antagonists. J. Biol. Chem. (2005) 280:9833-9841, the entire contents of which are incorporated herein by this reference.

Compounds selective for the S1P-1 receptor can be agonists of the S1P-1receptor, significantly weaker agonists of one or more other receptors and/or antagonists of one or more other receptors. The terms “agonist” or “S1P-1 receptor agonist” as used herein include the compounds described herein which bind to and/or agonize the S1P-1 receptor. The EC₅₀ of a compound is determined using the ³⁵S-GTPγS binding assay, as described in WO 03/061567, the entire contents of which are incorporated herein by reference. For example, compound 7a had an EC₅₀ of 6.9 nM.

In one embodiment, the S1P receptor agonists have an IC₅₀ for the S1P-1 receptor of about 100 nM-0.25 nM, about 50 nM-0.25 nM, about 25 nM-0.5 nM, about 100 nM or less, about 75 nM or less, about 50 nM or less, about 40 nM or less, about 30 nM or less, about 20 nM or less, about 10 nM or less, about 5 nM or less, about 1 nM or less, about 0.5 nM or less, or about 0.25 nM or less. The compounds' IC₅₀ for the S1P-1 receptor can be measured using the binding assays described in Example 13 or those described in WO 03/061567.

Ranges intermediate to the above recited values are also intended to be part of this invention. For example, ranges using a combination of any of the above recited values as upper and/or lower limits are intended to be included.

In a further embodiment, the S1P receptor agonist has an IC₅₀ value for the S1P-3 receptor of about 10 nM-10,000 nM, about 100 nM-5000 nM, about 100 nM-3000 nM, about 10 nM or greater, about 20 nM or greater, about 40 nM or greater, about 50 nM or greater, about 75 nM or greater, or about 100 nM or greater. In another embodiment, the S1P compound of the invention binds the S1P-3 receptor with an IC₅₀ of 1000 nM or greater, 2000 nM or greater, 3000 nM or greater, 5000 nM or greater, 10,000 nM or greater. The IC₅₀ for of S1P-3 receptor can be measured using the binding assays described herein or those described in WO 03/061567.

In addition, it should be understood that the ranges intermediate to the above recited values are also intended to be part of this invention. For example, ranges using a combination of any of the above recited values as upper and/or lower limits are intended to be included.

In yet another embodiment, the S1P receptor agonists described herein have an IC₅₀ value for the S1P-1 receptor that is about 5-fold lower, about 10-fold lower, about 20-fold lower, about 50-fold lower, about 100-fold lower, about 200-fold lower, about 500-fold lower or about 1000-fold lower than their IC₅₀ value for the S1P-3 receptor.

Ranges intermediate to the above recited values are also intended to be part of this invention. For example, ranges using a combination of any of the above recited values as upper and/or lower limits are intended to be included.

The ability of several of the compounds described herein to bind to the S1P-1 or S1P-3 receptor was tested as follows.

For the membrane preparation, plasmid DNA was transfected into HEK 293 T cells using the FuGENE 6 transfection protocol Briefly, subconfluent monolayers of HEK 293 T cells were transfected with the DNA mixture containing FuGENE 6 (using a 1:3 ratio). The dishes containing the cells were then placed in a tissue culture incubator (5% CO₂, 37° C.). The cells were harvested 48 hours after addition of the DNA by scraping in HME buffer (in mM: 20 HEPES, 5 MgCl₂, 1 EDTA, pH 7.4, 1 mM PMSF) containing 10% sucrose on ice, and disrupted using aDounce homogenizer. After centrifugation at 800×g, the supernatant was diluted with HME without sucrose and centrifuged at 17,000×g for 1 hour. This crude membrane pellet was resuspended in HME with sucrose, aliquoted, and snap-frozen by immersion in liquid nitrogen. The membranes were stored at −70° C. Protein concentration was determined spectroscopically by Bradford protein assay.

For the binding assay, [³³P]sphingosine 1-phosphate (obtained from American Radiolabeled Chemicals, Inc) was added to membranes in 200 μL in 96-well plates with assay concentrations of 2.5 pM [³³P]sphingosine 1-phosphate, 4 mg/mL BSA, 50 mM HEPES, pH 7.5, 100 mM NaCl, 5 mM MgCl2, and 5 μg of protein. Binding was performed for 60 minutes at room temperature with gentle mixing and terminated by collecting the membranes onto GF/B filter plates. After drying the filter plates for 10 minutes, 50 μL of Microscint 40 was added to each well, and filter-bound radionuclide was measured on a Packard Top Count. Nonspecific binding was defined as the amount of radioactivity remaining in the presence of excess of unlabeled S1P. The results for the foregoing binding assays are presented in Table 2 provided below.

TABLE 2
IC50 Values for Binding to S1P1 or S1P3 Receptors

| | IC50 | IC50 | IC50/IC50 |
|---|---|---|---|
| Structure | (S1P-1) | (S1P-3) | (S1P-3/S1P-1) |
| 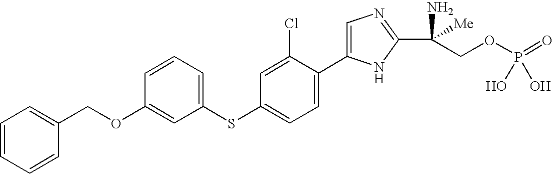 | **** | *** | +++ |
| 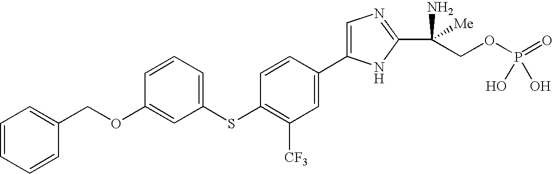 | **** | ** | +++ |
| 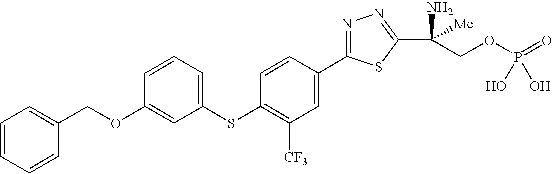 | **** | * | |
| 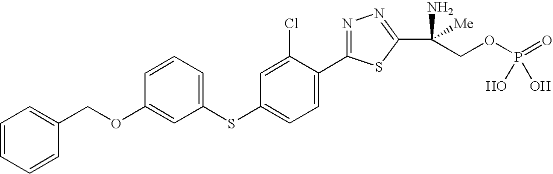 | **** | *** | ++ |
| 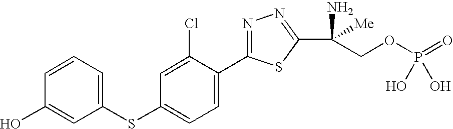 | *** | * | |

Key IC₅₀
* very low receptor binding affinity (IC₅₀ > 10,000 nM)
** low receptor binding affinity (10,000 nM ≧ IC₅₀ > 1,000 nM)
*** moderate receptor binding affinity (1,000 nM ≧ IC₅₀ > 100 nM)
**** high receptor binding affinity (100 nM ≧ IC₅₀ ≧ 0.001 nM)
+ moderate receptor selectivity (>100 fold)
++ good receptor selectivity (>100-1,000 fold)
+++ great receptor selectivity (>1,000 fold)

### Methods of Using Invention Compounds

The compounds of the invention have been determined to be useful in the treatment of S1P associated disorders. Accordingly, in one embodiment, the invention relates to a method for treating a subject suffering from a S1P associated disorder, comprising administering to a subject an effective amount of a compound of the invention; that is, a compound of formula I or compounds otherwise described herein, such that the subject is treated for a S1P associated disorder.

The term “S1P associated disorder” includes disorders, diseases or conditions which are associated with or caused by a misregulation in S1P receptor function and/or signaling or S1P receptor ligand function. The term also includes diseases, disorders or conditions which can be treated by administering to a subject an effective amount of a S1P receptor agonist. Such disorders include disorders that are associated with an inappropriate immune response and conditions associated with an overactive immune response, e.g., autoimmune diseases.

“Treatment”, or “treating” as used herein, is defined as the application or administration of a therapeutic agent such as a compound of formula I to a subject who has a S1P associated disorder as described herein, with the purpose to cure, heal, alleviate, delay, relieve, alter, remedy, ameliorate, improve or affect the disease or disorder, or symptoms of the disease or disorder. The term “treatment” or “treating” is also used herein in the context of administering agents prophylactically.

An additional embodiment of the invention pertains to a method for treating a subject suffering from a S1P associated disorder, comprising administering to a subject a compound, such that the subject is treated for a S1P associated disorder by a compound of the invention; that is, a compound of formulae I or compounds otherwise described herein.

The present invention is also directed to a method of selectively treating a S1P associated disorder, comprising administering to a subject an effective amount of a compound of the invention or compounds otherwise described herein, such that the subject is selectively treated for a S1P associated disorder. In certain embodiments, the S1P associated disorder is a S1P-1 associated disorder. In a particular embodiment, the S1P-1 associated disorder is selectively treated as compared with a S1P-3 associated disorder.

Another embodiment of the invention is a method of selectively treating a S1P associated disorder, comprising administering to a subject a compound, such that the subject is selectively treated for a sphingosine 1-phosphate associated disorder by a compound of the invention or compounds otherwise described herein. In certain embodiments, the S1P associated disorder is a S1P-1 associated disorder. In a particular embodiment, the S1P-1 associated disorder is selectively treated as compared with a S1P-3 associated disorder.

In another embodiment, the present invention provides a method of treating a condition associated with an activated immune system. Such diseases or disorders include rejection of transplanted organs, tissue or cells; graft-versus-host diseases brought about by transplantation; autoimmune syndromes including rheumatoid arthritis; systemic lupus erythematosus; antiphospholipid syndrome; Hashimoto's thyroiditis; lymphocytic thyroiditis; multiple sclerosis; myasthenia gravis; type I diabetes; uveitis; episcleritis; scleritis; Kawasaki's disease, uveo-retinitis; posterior uveitis; uveitis associated with Behcet's disease; uveomeningitis syndrome; allergic encephalomyelitis; chronic allograft vasculopathy; post-infectious autoimmune diseases including rheumatic fever and post-infectious glomerulonephritis; inflammatory and hyperproliferative skin diseases; psoriasis; psoriatic arthritis; atopic dermatitis; myopathy; myositis; osteomyelitis; contact dermatitis; eczematous dermatitis; seborrhoeic dermatitis; lichen planus; pemphigus; bullous pemphigoid; epidermolysis bullosa; urticaria; angioedema; vasculitis; erythema; cutaneous eosinophilia; acne; scleroderma; alopecia areata; keratoconjunctivitis; vernal conjunctivitis; keratitis; herpetic keratitis; dystrophia epithelialis corneas; corneal leukoma; ocular pemphigus; Mooren's ulcer; ulcerative keratitis; scleritis; Graves' ophthalmopathy; Vogt-Koyanagi-Harada syndrome; sarcoidosis; pollen allergies; reversible obstructive airway disease; bronchial asthma; allergic asthma; intrinsic asthma; extrinsic asthma; dust asthma; chronic or inveterate asthma; late asthma and airway hyper-responsiveness; bronchiolitis; bronchitis; endometriosis; orchitis; gastric ulcers; ischemic bowel diseases; inflammatory bowel diseases; necrotizing enterocolitis; intestinal lesions associated with thermal burns; coeliac disease; proctitis; eosinophilic gastroenteritis; mastocytosis; Crohn's disease; ulcerative colitis; vascular damage caused by ischemic diseases and thrombosis; atherosclerosis; fatty heart; myocarditis; cardiac infarction; aortitis syndrome; cachexia due to viral disease; vascular thrombosis; migraine; rhinitis; eczema; interstitial nephritis; IgA-induced nephropathy; Goodpasture's syndrome; hemolytic-uremic syndrome; diabetic nephropathy; glomerulosclerosis; glomerulonephritis; tubulointerstitial nephritis; interstitial cystitis; multiple myositis; Guillain-Barre syndrome; Meniere's disease; polyneuritis; multiple neuritis; myelitis; mononeuritis; radiculopathy; hyperthyroidism; Basedow's disease; thyrotoxicosis; pure red cell aplasia; aplastic anemia; hypoplastic anemia; idiopathic thrombocytopenic purpura; autoimmune hemolytic anemia; autoimmune thrombocytopenia; agranulocytosis; pernicious anemia; megaloblastic anemia; anerythroplasia; osteoporosis; fibroid lung; idiopathic interstitial pneumonia; dermatomyositis; leukoderma vulgaris; ichthyosis vulgaris; photoallergy sensitivity; cutaneous T cell lymphoma; polyarteritis nodosa; Huntington's chorea; Sydenham's chorea; myocardosis; myocarditis; scleroderma; Wegener's granuloma; Sjogren's syndrome; adiposis; eosinophilic fascitis; lesions of gingiva, periodontium, alveolar bone, substantia ossea dentis; male pattern alopecia or alopecia senilis; muscular dystrophy; pyoderma; Sezary's syndrome; hypophysiti's; chronic adrenal insufficiency; Addison's disease; ischemia-reperfusion injury of organs which occurs upon preservation; endotoxin shock; pseudomembranous colitis; colitis caused by drug or radiation; ischemic acute renal insufficiency; chronic renal insufficiency; lung solid cancer; malignancy of lymphoid origin; acute or chronic lymphocytic leukemias; lymphoma; psoriasis; pulmonary emphysema; cataracts; siderosis; retinitis pigmentosa; senile macular degeneration; vitreal scarring; corneal alkali burn; dermatitis erythema; ballous dermatitis; cement dermatitis; gingivitis; periodontitis; sepsis; pancreatitis; peripheral artery disease; carcinogenesis; solid cancer tumors; metastasis of carcinoma; hypobaropathy; autoimmune hepatitis; primary biliary cirrhosis; sclerosing cholangitis; partial liver resection; acute liver necrosis; cirrhosis; alcoholic cirrhosis; hepatic failure; fulminant hepatic failure; late-onset hepatic failure; “acute-on-chronic” liver failure.

As used herein, the term “subject” includes warm-blooded animals, e.g., mammals, including humans, cats, dogs, horses, bears, lions, tigers, ferrets, rabbits, mice, cows, sheep, pigs, etc. In a particular embodiment, the subject is a primate. In a specific embodiment, the primate is a human.

As used herein, the term “administering” to a subject includes dispensing, delivering or applying a compound of the invention in a pharmaceutical formulation (as described herein), to a subject by any suitable route for delivery of the compound to the desired location in the subject, including delivery by either the parenteral or oral route, intramuscular injection, subcutaneous/intradermal injection, intravenous injection, buccal administration, topical delivery, transdermal delivery and administration by the rectal, colonic, vaginal, intranasal or respiratory tract route.

As used herein, the term “effective amount” includes an amount effective, at dosages and for periods of time necessary, to achieve the desired result; that is, sufficient to treat the condition in a subject. An effective amount of a compound of the invention, as defined herein, may vary according to factors such as the disease state, age, and weight of the subject, and the ability of the compound to elicit a desired response in the subject. Dosage regimens may be adjusted to provide the optimum therapeutic response. An effective amount is also one in which any toxic or detrimental effects (such as side effects) associated with administration of the compound are outweighed by the therapeutically beneficial effects.

A therapeutically effective amount of a compound of the invention (i.e., an effective dosage) may range from about 0.001 to 30 mg/kg body weight, for example, about 0.01 to 25 mg/kg body weight, for example, about 0.1 to 20 mg/kg body weight. It is to be understood that all values and ranges between those listed are intended to be encompassed by the present invention. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound of the invention can include a single treatment or, for example, can include a series of treatments. It will also be appreciated that the effective dosage of the compound used for treatment may increase or decrease over the course of a particular treatment.

The methods of the invention further include administering to a subject a therapeutically effective amount of a compound of the invention in combination with another pharmaceutically active compound known to treat the disease or condition, e.g., an immunomodulatory agent or an anti-inflammatory agent. Pharmaceutically active compounds that may be used depend upon the condition to be treated, but include as examples cyclosporin, rapamycin, FK506, methotrexate, etanercept, infliximab, adalimumab, non-steroidal anti-inflammatory agents, cyclooxygenase-2-inhibitors, such as celecoxib and rofecoxib, and corticosteroids. Other suitable compounds can be found in Harrison's Principles of Internal Medicine, Thirteenth Edition, Eds. T. R. Harrison et al. McGraw-Hill N.Y., N.Y.; and the Physicians Desk Reference 50th Edition 1997, Oradell N.J., Medical Economics Co., the complete contents of which are expressly incorporated herein by reference. The compound of the invention and the additional pharmaceutically active compound may be administered to the subject in the same pharmaceutical composition or in different pharmaceutical compositions (at the same time or at different times).

### Pharmaceutical Compositions Comprising Invention Compounds

The present invention also provides pharmaceutically acceptable formulations

and compositions comprising one or more compounds of the invention; that is, compounds of formula I or compounds otherwise described herein. In certain embodiments, the compound of the invention is present in the formulation in a therapeutically effective amount; that is, an amount effective to treat a S1P-associated disorder.

Accordingly, in one embodiment, the invention pertains to a pharmaceutical composition comprising a therapeutically effective amount of a compound of the invention; that is, compounds of formula I or compounds otherwise described herein, and a pharmaceutically acceptable carrier.

In another embodiment, the invention is directed to a packaged pharmaceutical composition comprising a container holding, a therapeutically effective amount of a compound of the invention; that is, compounds of formula I or compounds otherwise described herein; and instructions for using the compound to treat a sphingosine 1-phosphate associated disorder in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions can contain information pertaining to the compound's ability to perform its intended function, e.g., treating, preventing, or reducing a SIP-associated disorder in a subject.

Another embodiment of the invention relates to a packaged pharmaceutical

composition comprising a container holding a therapeutically effective amount of a compound of the invention; that is, a compound of formula I or compounds otherwise described herein, and instructions for using the compound to selectively treat a S1P-associated disorder in a subject.

Such pharmaceutically acceptable formulations typically include one or more compounds of the invention as well as one or more pharmaceutically acceptable carriers and/or excipients. As used herein, “pharmaceutically acceptable carrier” includes any and all solvents, dispersion media, coatings, antibacterial and antifungal agents, isotonic and absorption delaying agents, and the like that are physiologically compatible. The use of such media and agents for pharmaceutically active substances is well known in the art. Except insofar as any conventional media or agent is incompatible with the compounds of the invention, use thereof in the pharmaceutical compositions is contemplated.

Supplementary pharmaceutically active compounds known to treat transplant or autoimmune disease, i.e., immunomodulatory agents and anti-inflammatory agents, as described above, can also be incorporated into the compositions of the invention. Suitable pharmaceutically active compounds that may be used can be found in Harrison's Principles of Internal Medicine.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injection include sterile aqueous

solutions (where water soluble) or dispersions, or sterile powders for the extemporaneous preparation of sterile injectable solutions or dispersions. For intravenous administration, suitable carriers include physiological saline, bacteriostatic water, Cremophor EI™ (BASF, Parsippany, N.J.) or phosphate buffered saline (PBS). In all cases, the pharmaceutical composition must be sterile and should be fluid to the extent that easy syringability exists. It must also be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the compound of the invention in the required amount in an appropriate solvent with one or a combination of the ingredients enumerated above, as needed, followed by filtered sterilization. Generally, dispersions are prepared by incorporating the compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the compound plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the compound of the invention can be incorporated with excipients and used in the form of tablets, troches, or capsules. Oral compositions can also include an enteric coating. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds of the invention are delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds of the invention are formulated into ointments, salves, gels, or creams as generally known in the art.

The present pharmaceutical compositions can also be prepared in the form of suppositories (with conventional suppository bases such as cocoa butter and other glycerides) or retention enemas for rectal delivery.

In one embodiment, the compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811, U.S. Pat. No. 5,455,044and U.S. Pat. No. 5,576,018, and U.S. Pat. No. 4,883,666, the contents of all of which are incorporated herein by reference.

The compounds of the invention can also be incorporated into pharmaceutical compositions which allow for the sustained delivery of the compounds to a subject for a period of at least several weeks to a month or more. Such formulations are described in published PCT application no. WO 02/74247, incorporated herein by reference.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of a compound of the invention calculated to produce the desired therapeutic effect in association with the required pharmaceutical carrier. The specification for the unit dosage forms of the invention are dictated by and directly dependent on the unique characteristics of the compound and the particular therapeutic effect to be achieved, and the limitations inherent in the art of compounding such compounds for the treatment of individuals.

This invention is further illustrated by the following examples, which should not be construed as limiting. The contents of all references, patents, patent applications cited throughout this application are incorporated herein by reference. It should be understood that the use of any of the compounds described herein are within the scope of the present invention and are intended to be encompassed by the present invention and are expressly incorporated herein for all purposes.

### EXAMPLES

### Example 1

### General protocol for synthesis of substituted 1-(4-(phenylthio)phenyl)ethanone

A mixture of substituted mercaptophenol (1.0 equiv), substituted 1-(4-fluorophenyl)ethanone 1 (1.0 equivalents), and K₂CO₃ (2.0 equiv) in DMF was heat at 50° C. for 3-18 hours under a nitrogen atmosphere. Benzyl bromide (1.0 equivalent) was then added, and the resulting mixture was stirred for additional 3 hours at 60° C. The mixture was allowed to cool to room temperature, and then was diluted with ethyl acetate (EtOAc) and washed with water (2 times), and brine (1 time). The organic layer was dried with MgSO₄ and concentrated under reduced pressure. The product was purified by silica gel column chromatography using the Combi-Flash system as required.

### 1-(4-(3-(benzyloxy)phenylthio)-2-chlorophenyl)ethanone (2a)

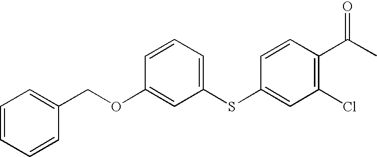

The title compound was purified by silica gel column chromatography using the Combi-Flash system (Hex:EtOAc) to give 4.10 g (70%) of a yellowish oil. Proton NMR and LC analyses confirmed the desired product with purity greater than 95%. TLC (1:4EtOAc:Hex), R_{f}=0.6; ¹H NMR (400 MHz, CDCl₃) δ 7.47 (d, 1H, J=8.4 Hz), 7.29-7.43 (m, 6H), 7.19 (d, 1H, J=2.0 Hz), 7.05-7.10 (m, 3H), 6.98-7.02 (m, 1H), 5.06 (s, 2H), 2.63 (s, 3H).

### 1-(4-(3-(benzyloxy)phenylthio)-3-(trifluoromethyl)phenyl)ethanone (2b)

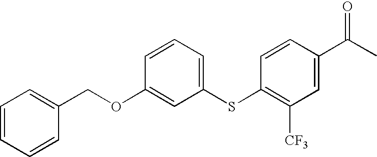

The title compound was purified by silica gel column chromatography using the Combi-Flash system (Hex:EtOAc) to give 1.92 g (60%) of a yellowish oil. Proton NMR and LC analyses confirmed the desired product with purity greater than 95%. TLC (1:4 EtOAc:Hex), R_{f}=0.6; ¹H NMR (400 MHz, CDCl_{3) δ} 8.02 (d, 1H, J=1.6 Hz), 7.81 (dd, 1H, J=8.4 Hz, J=1.6 Hz), 7.30-7.43 (m, 6H), 7.11-7.15 (m, 2H), 7.03-7.08 (m, 2H), 5.06 (s, 2H), 2.7=58 (s, 3H).

### Example 2

### General Protocol for Synthesis of Substituted phenyl-imidazole Scaffold

To a solution of the substituted acetophenone 2 (1.0 equivalent) in MeOH/CH₂Cl₂ (1:4) under a nitrogen atmosphere was added tetrabutyl ammonium tribromide (Bu₄NBr₃) (1.0 equivalents). The reaction mixture was stirred at room temperature for 2-18 hours. The reaction was monitored by TLC or LC to confirm completion. The solvent removed in vacuo and the crude product was either carried forward without further purification or was purified by silica gel column chromatography using the Combi-Flash system (Hex:EtOAc).

A mixture of the desired bromo-acetophenone 3 (from last step, 1.0 equivalent), Boc-α-MeSer (1.0 equiv), and Cs₂CO₃ (0.6 equivalent) was stirred in DMF for 1-2 hours. The reaction mixture was diluted with EtOAc and washed with water (2×), and saturated brine (1×) to remove excess DMF and CsBr. The organic layer was dried over anhydrous MgSO₄ and the solvent was removed at reduced pressure. TLC generally showed a spot to spot conversion of the starting material to product ester.

To the thus-obtained ester was then added excess ammonium acetate (10 equivalents), and the mixture was suspended in toluene and refluxed for 3-6 hours using a Dean-Stark apparatus. The mixture was diluted with EtOAc and washed with water (2×), and brine (1×). The organic layer was dried over anhydrous MgSO₄ and the solvent was removed at reduced pressure. The product was purified by silica gel column chromatography using the Combi-Flash system (Hex:EtOAc).

### (R)-tert-butyl 2-(4-(4-(3-(benzyloxy)phenylthio)-2-chlorophenyl)-1H-imidazol-2-yl)-1-hydroxypropan-2-ylcarbamate (4a)

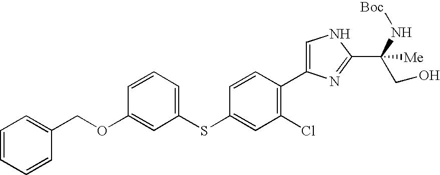

The title compound was purified by silica gel column chromatography using the Combi-Flash system (Hex:EtOAc) as yellow solid in 27% (120 mg). LC-MS analyses confirmed the desired product with purity greater than 90%. TLC (1:1 EtOAc:Hex), R_{f}=0.3; MS (ESI, M+H⁺)=566.05

### (R)-tert-butyl 2-(4-(4-(3-(benzyloxy)phenylthio)-S-(trifluoromethyl)phenyl)-1H-inidazol-2-yl)-1-hydroxypropan-2-ylcarbamate (4b)

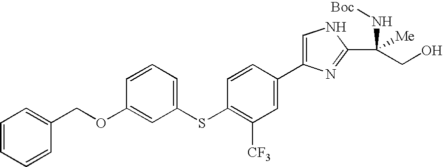

The title compound was purified by silica gel column chromatography using the Combi-Flash system (Hex:EtOAc) to give 320 mg (80%) as a yellow solid. LC-MS analyses confirmed the desired product with purity greater than 95%. TLC (1:1 EtOAc:Hex), R_{f}=0.3; MS (ESI, M+H⁺)=600.07

### Example 4

### General Protocol for Deprotection of the Amino Group

To a solution of the Boc-protected precursor 4 (1.0 equiv) in CH₂Cl₂ was added TFA (25% by volume). The reaction mixture was stirred at room temperature for 1-2 hours then evaporated to dryness under reduced pressure to afford the desired product. The final product was purified by reverse-phase preparative HPLC of the corresponding TFA salt in 60-70% yield.

### (R)-2-amino-2-(4-(4-(S-(benzyloxy)phenylthio)-2-chlorophenyl)-1H-imidazol-2-yl)propan-1-ol (5a)

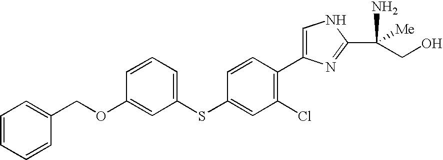

The title compound was purified as the TFA salt by reverse phase preparative HPLC, followed by lyophilization to give the 14 mg of the product as a white solid (60%). MS (ESI, M+H⁺)=461.1; ¹H NMR (400 MHz, DMSO-d₆) δ 8.42 (br s, 2H), 8.13 (br s, 1H), 7.81 (br s, 1H), 7.28-7.42 (m, 7H), 6.90-7.02 (m, 3H), 5.08 (s, 2H), 3.77 (d, 1H, J=10.4 Hz), 3.66 (d, 1H, J=10.4 Hz), 1.56 (s, 3H).

### (R)-2-amino-2-(4-(4-(3-(benzyloxy)phenylthio)-3-(trifluoromethyl)phenyl)-1H-imidazol-2-yl)propan-1-ol (5b)

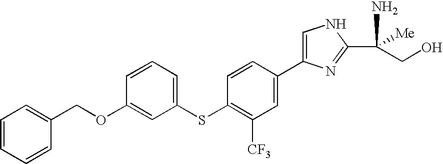

The title compound was purified as the TFA salt by reverse phase preparative HPLC, followed by lyophilization to give the 45 mg of the product as a white solid (70%). MS (ESI, M+H⁺)=500.06; ¹H NMR (400 MHz, DMSO-d₆) δ 8.44 (br s, 3H), 8.26 (br s, 1H), 7.97 (d, 1H, J=7.6 Hz), 7.93 (br s, 1H), 7.27-7.43 (m, 6H), 6.98 (dd, 1H, J=8.4 Hz, J=2.4 Hz), 6.82-6.89 (m, 2H), 5.70 (br s, 1H), 5.06 (s, 2H), 3.75 (d, 1H, J=10.8 Hz), 3.65 (d, 1H, J=10.8 Hz), 1.54 (s, 3H).

### Example 5

### Preparation of Phenyl-thiadiazole Analogs

### 4-Methoxybenzyl 4-(3-(benzyloxy)phenylthio)-3-(trifluoromethyl)benzoate (7a)

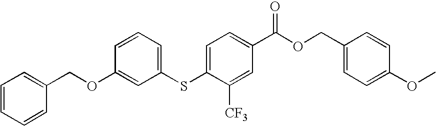

A suspension of 4-methoxybenzyl 4-fluoro-3-(trifluoromethyl)benzoate (602 mg, 1.83 mmol), potassium carbonate (555 mg, 4.03 mmol) and 3-mercaptophenol (241 mg, 96%, 1.83 mmol) in DMF (10 mL) was stirred at 50° C. for overnight. Benzyl bromide (638 mL, 3.66 mmol) was then added drop-wise followed by raising the temperature to 65° C. for 3 hours. The reaction was cooled to room temperature and diluted with ethyl acetate (30 mL) and washed with water (10 mL) followed by brine (2×10 mL). The organic layer was dried over Na₂SO₄ and concentrated under vacuum to provide a crude product, which was further purified on a silica gel column, washed with ethyl acetate/hexanes (0-20%, v/v) and afforded the title compound (717 mg, 75%). TLC (EtOAc:Hex, 1:5), R_{f}=0.35; ¹H NMR (400 MHz, CDCl₃) δ 8.29 (d, 1H, J=1.6 Hz), 7.89 (dd, 1H, J=8.4 Hz, J=1.6 Hz), 7.39-7.29 (m, 8H), 7.10 (dd, 2H, J=7.4 Hz, J=1.6 Hz), 7.02 (t, 2H, J=8.0 Hz), 6.90 (dt, 2H, J=8.8 Hz, J=2 Hz), 5.28 (s, 2H), 5.03 (s, 2H), 3.80 (s, 3H).

### 4-Methoxybenzyl 4-(3-(benzyloxy)phenylthio)-2-chlorobenzoate (7b)

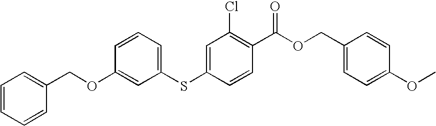

The title compound was prepared analogously to 7a by using 4-methoxybenzyl 2-chloro-4-fluorobenzoate as a starting material. TLC (EtOAc:Hex, 1:5), R_{f}=0.25; ¹H NMR (400 MHz, CDCl₃) δ 7.71 (d, 2H, J=8.0 Hz), 7.41-7.28 (m, 7H), 7.20 (d, 1H, J=2.0 Hz), 7.07-6.98 (m, 4H), 6.91-6.88 (dt, 2H, J=8.8 Hz, J=2.4 Hz), 5.27 (s, 2H), 5.04 (s, 2H), 3.80 (s, 3H).

### 4-(3-(Benzyloxy)phenylthio)-3-(trifluoromethyl)benzohydrazide (8a)

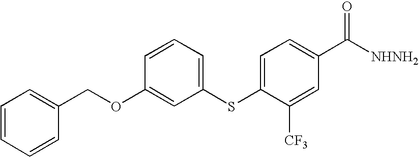

A mixture of 4-methoxybenzyl 4-(3-(benzyloxy)phenylthio)-3-(trifluoromethyl)-benzoate (7a, 397 mg), hydrazine (0.6 mL) and ethylene glycol (10 mL) was heated at 140° C. with stirring for 2 h. Cooled to room temperature, the reaction was poured into cold water (10 mL) and the title compound was collected as white crystal-like solid (285 mg, 90%). MS (ESI, MH⁺)=419.03.

### 4-(3-(Benzyloxy)phenylthio)-2-chlorobenzohydrazide (8b)

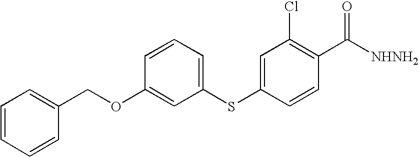

The title compound was prepared analogously to 8a by using 4-methoxybenzyl 4-(3-(benzyloxy)phenylthio)-2-chlorobenzoate (7b) as a starting material in 71% yield. MS (ESI, MH⁺)=385.06.

### (R)-t-Butyl 4-(2-(4-(3-(benzyloxy)phenylthio)-3-(trifluoromethyl)benzoyl)hydrazine-carbonyl)-2,2,4-trimethyloxazolidine-3-carboxylate (10a)

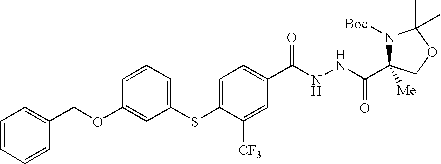

To a solution of (R)-3-Boc-2,2,4-trimethyloxazolidine-4-carboxylic acid (60 mg, 0.23 mmol) in CH₂Cl₂/DMF (2:1, 3 mL) was added HATU (87 mg, 0.27 mmol) and DIEA (0.2 mL, 1.15 mmol). The resultant was stirred at room temperature for 10 min and then was added 4-(3-(benzyloxy)phenylthio)-3-(trifluoromethyl)benzohydrazide (8a, 96 mg, 0.23 mmol). The reaction was continuously stirred for another 30 min and then directly chromatographed on a silica gel column eluted with ethyl acetate/hexane (0˜30%, v/v) to afford the title compound (145 mg, 95%). TLC (EtOAc:Hex, 1:5), R_{f}=0.20; MS (ESI, MH⁺)=659.85; ¹H NMR (400 MHz, CDCl₃) δ 8.10 (d, 1H, J=1.6 Hz), 7.66 (dd, 1H, J=8.0 Hz, J=1.6 Hz), 7.41-7.31 (m, 6H), 7.11-7.09 (m, 2H), 7.06-7.03 (m, 2H), 5.05 (s, 2H), 4.60-4.40 (br s, 1H), 3.75 (br s, 1H), 1.58 (s, 3H), 1.51 (s, 9H).

### (R)-t-Butyl 4-(2-(4-(3-(benzyloxy)phenylthio)-2-chlorobenzoyl)hydrazinecarbonyl)-2,2,4-trimethyloxazolidine-3-carboxylate (10b)

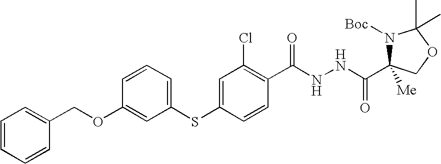

The title compound was prepared analogously to 10a by using 4-(3-(Benzyloxy)phenylthio)-2-chlorobenzohydrazide (8b) as a starting material in 90% yield. TLC (EtOAc:Hex, 1:5), R_{f}=0.15; MS (ESI, MH⁺)=625.91; ¹H NMR (400 MHz, CDCl₃) δ 7.71 (d, 1H, J=4.8 Hz), 7.42-7.30 (m, 6H), 7.18 (d, 1H, J=2.0 Hz), 7.10 (dd, 1H, J=8.4 Hz, J=2.0 Hz), 7.08-7.06 (m, 2H), 7.02-7.00 (m, 1H), 5.05 (s, 2H), 4.60-4.30 (br, 1H), 3.77 (br, 1H), 1.58 (s, 3H), 1.51 (s, 9H).

### (R)-t-Butyl 4-(5-(4-(3-(benzyloxy)phenylthio)-3-(trifluoromethyl)phenyl)-1,3,4-thiadiazol-2-yl)-2,2,4-trimethyloxazolidine-3-carboxylate (11a)

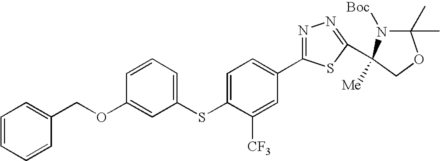

The suspension of 10a (145 mg, 0.22 mmol) and Lawesson's reagent (266 mg, 0.66 mg) in toluene (5 mL) was heated at 85° C. for 2 h with stirring. After cooling down to room temperature, the supernatant was directly chromatographed on a silica gel column eluted with ethyl acetate/hexane (0-15%) to afford the title compound (123 mg, 85%). MS (ESI, MH⁺)=658.16.

### (R)-t-Butyl 4-(5-(4-(3-(benzyloxy)phenylthio)-2-chlorophenyl)-1,3,4-thiadiazol-2-yl)-2,2,4-trimethyloxazolidine-3-carboxylate (11b)

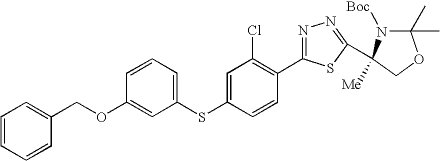

The title compound was prepared analogously to 11a by using (R)-t-Butyl 4-(2-(4-(3-(benzyloxy)phenylthio)-2-chlorobenzoyl)hydrazinecarbonyl)-2,2,4-trimethyloxazoli-dine-3-carboxylate (10b) as a starting material in 83% yield. MS (ESI, MH⁺)=624.14

### (S)-2-Amino-2-(5-(4-(3-(benzyloxy)phenylthio)-3-(trifluoromethyl)phenyl)-1,3,4-thiadiazol-2-yl)propanol (12a)

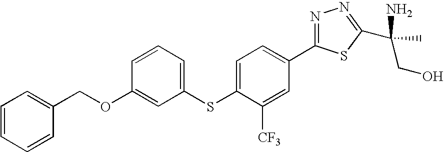

A solution of (R)-t-Butyl 4-(5-(4-(3-(benzyloxy)phenylthio)-3-(trifluoromethyl)-phenyl)-1,3,4-thiadiazol-2-yl)-2,2,4-trimethyloxazolidine-3-carboxylate (11a, 123 mg, 0.18 mmol) in methanol (7 mL) was heated at 70° C. for 2 hours. After cooling to room temperature, water (2 mL) was added and the reaction mixture was directly injected to preparative HPLC for purification with 30-90% acetonitrile-H₂O (0.1% TFA) in 15 minutes of gradient time as mobile phase to afford the title compound (97 mg, 72%) as bis-TFA salt. MS (ESI, MH⁺)=518.02; ¹H NMR (400 MHz, DMSO-d6) δ 8.89 (br, 3H), 8.30 (s, 1H), 8.12 (d, 1H, J=8.4 Hz), 7.47 (d, 1H, J=8.0 Hz), 7.43 (d, 2H, J=8.8 Hz), 7.37 (t, 2H, J=7.2 Hz), 7.33 (d, 1H, J=6.8 Hz), 7.26 (d, 1H, J=8.8 Hz), 7.20-7.16 (m, 2H), 7.14 (d, 1H, J=8.0 Hz), 6.16 (br s, 1H), 5.14 (s, 2H), 3.84 (dd, 1H, J=10.8 Hz, J=3.2 Hz), 3.77 (dd, 1H, J=10.8 Hz, J=3.2 Hz), 1.71 (s, 3H).

### (S)-2-Amino-2-(5-(4-(3-(benzyloxy)phenylthio)-2-chlorophenyl)-1,3,4-thiadiazol-2-yl)propanol (12b)

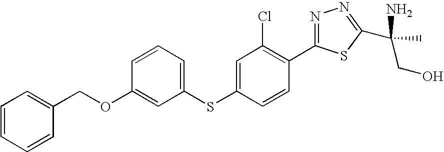

The title compound was prepared analogously to 12a by using (R)-t-Butyl 4-(5-(4-(3-(benzyloxy)phenylthio)-2-chlorophenyl)-1,3,4-thiadiazol-2-yl)-2,2,4-trimethyloxazoli-dine-3-carboxylate (11b) as a starting material in 77% yield (bis-TFA salt). MS (ESI, MH⁺)=484.01; ¹H NMR (400 MHz, DMSO-d6) δ 8.50 (br, 1H), 8.12 (d, 1H, J=8.0 Hz), 7.46-7.42 (m, 4H), 7.38 (t, 2H, J=7.2 Hz), 7.33-7.28 (m, 2H), 7.19 (s, 1H), 7.15 (t, 2H, J=7.2 Hz), 6.06 (br s, 1H), 5.15 (s, 2H), 3.84 (dd, 1H, J=10.8 Hz, J=5.2 Hz), 3.76 (dd, 1H, J=10.8 Hz, J=5.2 Hz), 1.70 (s, 3H).

### Example 6

### General Method for Phosphate Synthesis

The general method for the synthesis of the desired phosphates in illustrated in scheme 3. To a solution of unprotected or Boc-protected amino alcohol (1.0 equiv) in dry CH₂Cl₂ at room temperature was added excess diethyl chlorophosphate (5.0-20.0 equiv) and triethylamine (5.0-30.0 equiv) and the reaction was stirred at room temperature for 12-18 hours. The crude was then loaded onto a silica gel column chromatography, as is, to purify the desired phospho-diester. The phopho-diester intermediate was reacted with excess bromotrimethylsilane (10.0-20.0 equiv) in dry CH₂Cl₂ at room temperature, under an atmosphere of nitrogen; over a period of 5-8 hours afforded the final phosphate which was purified by reverse-phase preparative HPLC.

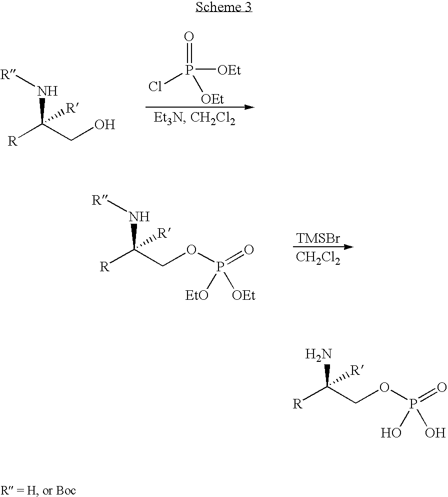

### (R)-2-amino-2-(4-(4-(3-(benzyloxy)phenylthio)-2-chlorophenyl)-1H-imidazol-2-yl)propyl dihydrogen phosphate

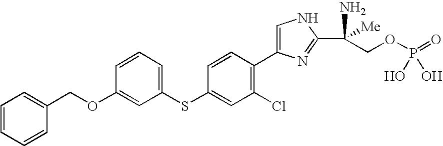

The final product was purified by reverse phase preparative HPLC, then lyophilized to dryness to obtain TFA salt of the product. The product was obtained as a white solid in 24% (10 mg) yield from the alcohol precursor. MS (ESI, M+H⁺)=546.3.

### (R)-2-amino-2-(4-(4-(3-(benzyloxy)phenylthio)-3-(trifluoromethyl)phenyl)-1H-imidazol-2-yl)propyl dihydrogen phosphate

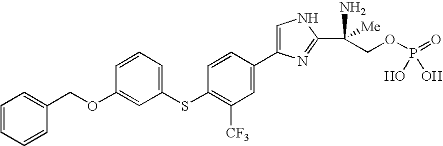

The final product was purified by reverse phase preparative HPLC, then lyophilized to dryness to obtain TFA salt of the product. The product was obtained as a white solid in 47% (45 mg) yield from the alcohol precursor. MS (ESI, M+H⁺)=580.07.

### (S)-2-Amino-2-(5-(4-(3-(benzyloxy)phenylthio)-3-(trifluoromethyl)phenyl)-1,3,4-thiadiazol-2-yl)propyl dihydrogen phosphate

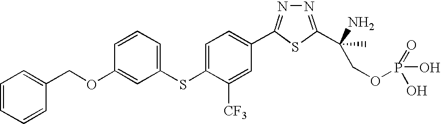

Yield: 6.8% (the low yield is due to the breakdown of the benzyl group in the deprotection step), MS (ESI, MH⁺)=597.98, purity 99% by HPLC.

### (S)-2-Amino-2-(5-(4-(3-(benzyloxy)phenylthio)-2-chlorophenyl)-1,3,4-thiadiazol-2-yl)propyl dihydrogen phosphate

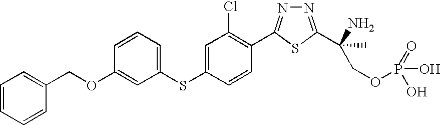

Yield: 6.5% (the low yield is due to the breakdown of the benzyl group in the deprotection step), MS (ESI, MH⁺)=563.97, purity 99% by HPLC.

### Example 7

### General Synthetic Strategy for Synthesis of phenyl-imidazole Analogs

The general approach for synthesis of various biaryl-thio-ethers 6 and 7 is described in Scheme 4. Reaction of substituted 3-mercaptophenol with substituted 1-fluoro-4-nitrobenzene 1 and benzyl bromide afforded the thio-ether-nitrobenzene intermediate 2. The thio-ether-nitrobenzene 2 was then converted to the thio-ether-aniline 3 using SnCl₁. Acylation of the thio-ether-aniline 3 with oxazolidine-carboxylic acid 4 under acid chloride condition afforded the amide 5. Deprotection of the protecting groups with para-toluenesulfonic acid (PTSA) provided the alcohol 6 in good yield. Alcohol 6 was then converted to phosphate 7 in two steps.

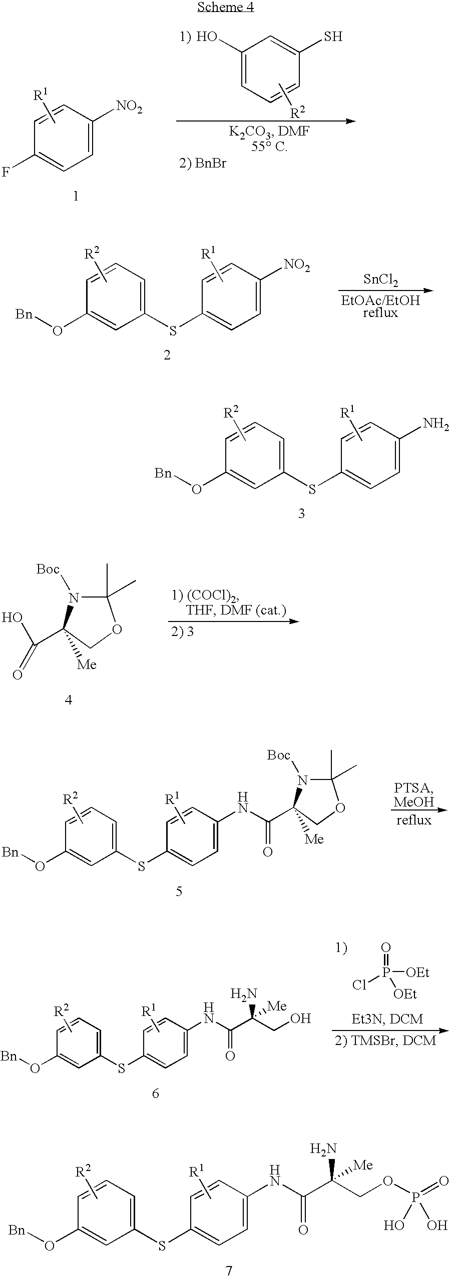

### Example 8

### General Protocol for Synthesis of Substituted 1-(4-(phenylthio)phenyl)ethanone

A mixture of substituted mercaptophenol (1.0 equivalent), substituted 1-(4-fluorophenyl)ethanone 1 (1.0 equivalent), and K₂CO₃ (2.0 equivalents) in DMF under nitrogen atmosphere was heat at 50° C. for 3-18 hours. To the reaction was then added BnBr (if necessary, 1.0 equivalent) and stirred for additional 3 hours at 60° C. The reaction cooling to room temperature then diluted with EtOAc and washed with H₂O (2×); saturated NaCl (1×), dried with MgSO₄. The organic layer was then concentrated under reduced pressure. The product was purified by silica gel column chromatography using the Combi-Flash system as required.

### (3-(Benzyloxy)phenyl)(3-chloro-4-nitrophenyl)sulfane (2a)

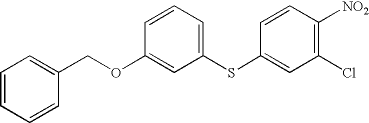

The product was purified by silica gel column chromatography using the Combi-Flash system (Hex:EtOAc) as yellowish oil in 37% (1.10 g). Proton NMR and LC analyses confirmed the desired product with purity greater than 95%. TLC (1:4 EtOAc:Hex), R_{f}=0.6; ¹H NMR (400 MHz, CDCl₃) δ 7.78 (d, 1H, J=8.4 Hz), 7.31-7.43 (m, 6H), 7.20 (d, 1H, J=2.0 Hz), 7.05-7.10 (m, 3H), 7.02 (dd, 1H, J=8.4 Hz, J=1.6 Hz), 5.08 (s, 2H).

### 4-(3-(Benzyloxy)phenylthio)-2-chloroaniline (3a)

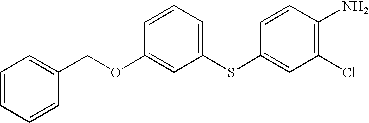

To a mixture of the nitro intermediate 2a (1.05 g, 1.0 equivalent) and SnCb (2.68 g, 5.0 equivalents) was added EtOH/EtOAc (1:1, 30 mL) then heated at 80° C. for 2 hours. The solvent was removed in vacuo and the product was purified by silica gel column chromatography using Combi-Flash system (Hex:EtOAc). The product was obtained as colorless oil in 88% (0.85 g) yield. TLC (1:4 EtOAc:Hex), R_{f}=0.4; ¹H NMR (400 MHz, CDCl₃) δ 7.41 (d, 1H, J=2.0 Hz), 7.28-7.40 (m, 6H), 7.20 (dd, 1H, J=8.0 Hz, J=2.0 Hz), 7.14 (t, 1H, J=7.4 Hz), 6.71-6.80 (m, 3H), 4.98 (s, 2H), 4.21 (br s, 2H).

### (R)-tert-Butyl 4-(4-(3-(benzyloxy)phenylthio)phenylcarbamoyl)-2,2,4-trimethyloxazolidine-3-carboxylate (5a)

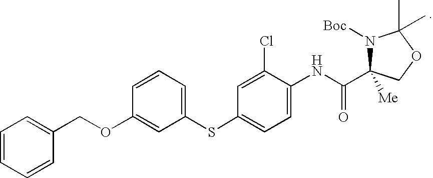

To a solution of (S)-3-(tert-butoxycarbonyl)-2,2,4-trimethyloxazolidine-4-carboxylic acid 4 (200 mg, 1.0 equivalent) in dry THF (6 mL) was added oxalyl chloride (1.2 equivalents) and catalytic amount of DMF (2 drops). The reaction was allowed to stir at room temperature for 20 minutes. To the reaction mixture was then added the aniline 3a (1.0 equivalent). The reaction was allowed to stir overnight. The solvent removed in vacuo and the crude product was purified by silica gel column chromatography using Combi-Flash system (Hex:EtOAc). The product was obtained as a colorless oil in 45% (200 mg) yield. TLC (1:4 EtOAc:Hex), R_{f}=0.6; ¹H NMR (400 MHz, CDCl₃) δ 8.42 (d, 1H, J=8.4 Hz), 7.24-7.42 (m, 8H), 7.21 (t, 1H, J=8.4 Hz), 6.80-6.92 (m, 2H), 5.01 (s, 2H), 3.89 (br s, 2H), 1.32-1.84 (m, 18H).

### (S)-2-Amino-N-(4-(3-(benzyloxy)phenylthio)phenyl)-3-hydroxy-2-methylpropanamide (6a)

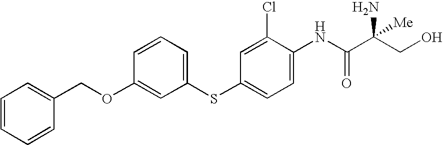

A solution of oxazolidine-amide 5a (200 mg) and PTSA mono-hydrate (652 mg, 10 equivalents) in MeOH (10 mL) was refluxed for overnight. The solvent was removed in vacuo and the product was purified by reverse phase preparative HPLC, then lyophilized to dryness to obtain TFA salt of the product. The solvent was obtained as a white solid in 65% yield (124 mg). MS (ESI, M+H⁺)=443.09; ¹H NMR (400 MHz, DMSO-d₆) δ 9.92 (s, 1H), 8.20 (s, 3H), 7.26-7.51 (m, 9H), 7.93-7.04 (m, 3H), 5.97 (br s, 1H), 5.09 (br s, 2H), 3.93 (dd, 1H, J=11.2, Hz, J=1.2 Hz), 3.69 (dd, 1H, J=11.2 Hz, J=1.2 Hz), 1.51 (s, 3H).

### Example 9

### General Method for Phosphate Synthesis

The general method for synthesis of the desired phosphates in illustrated in scheme 1. To a solution of amino alcohol 6a (1.0 equivalent) in dry CH₂Cl₂ at room temperature was added excess diethyl chlorophosphate (10.0 equivalents) and triethylamine (20.0 equivalents) and the reaction stirred for 12-18 hours. The solvent removed in vacuo and the crude phopho-diester intermediate was reacted with excess bromotrimethylsilane (20.0 equivalents) in dry CH₂Cl₂ at room temperature over a period of 5 hours to afford the final phosphate which was purified by reverse-phase preparative HPLC.

### (S)-2-Amino-3-(4-(3-(benzyloxy)phenylthio)phenylamino)-2-methyl-3-oxopropyl dihydrogen phosphate 7a

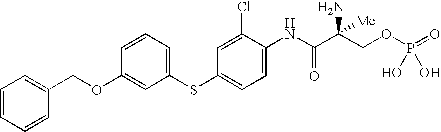

The final product was purified by reverse phase preparative HPLC, then lyophilized to dryness to obtain TFA salt of the product. The product was obtained as a white solid in 23% (10.4 mg) yield from the alcohol precursor. MS (ESI, M+H⁺)=523.03;

1. A compound of formula I 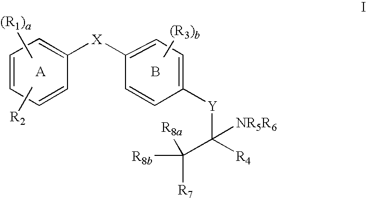
or a pharmaceutically acceptable salt thereof, wherein:
R₁ is halogen, cyano, alkyl, aryl, heteroaryl, cycloalkyl, heterocycloalkyl, aralkyl, heteroalkyl, —O-alkyl, —O-aryl, —O-heteroaryl, —S-alkyl, alkylene-O-alkyl, alkylene-CO₂H, alkylene-CO₂alkyl, alkylSO₂, alkylSO, aralkylSO₂, aralkylSO, alkylene-CO-amino, alkylene-CO-alkylamino, alkylene-CO-dialkylamino, alkylene-NH—CO₂H, alkylene-NH—CO₂alkyl-CO₂alkyl, —OH, —C(O)-alkyl, —C(O)O-alkyl, —CONH₂, —CO-alkylamino, —CO-dialkylamino, amino, alkylamino, or dialkylamino, any of which may be optionally substituted on carbon with 1, 2, or 3 groups selected from halo, alkyl, OH, or —O-alkyl;
R₂ is halogen, cyano, alkyl, aryl, heteroaryl, cycloalkyl, heterocycloalkyl, aralkyl, heteroalkyl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylSO₂, alkylSO, aralkylSO₂, aralkylSO, alkylene-CO-amino, alkylene-CO-alkylamino, alkylene-CO-dialkylamino, alkylene-NH—CO₂H, alkylene-NH—CO₂alkyl-CO₂alkyl, —OH, —C(O)-alkyl, —C(O)O-alkyl, —CONH2, —CO-alkylamino,
—CO-dialkylamino, amino, alkylamino, and dialkylamino, any of which may be optionally substituted on carbon with 1, 2, or 3 groups selected from halo, alkyl, OH, or —O-alkyl;
R₃ is halogen, cyano, alkyl, aryl, heteroaryl, cycloalkyl, heterocycloalkyl, aralkyl, heteroalkyl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl-CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;
a is 0, 1, 2, or 3;
b is 1, 2 or 3; 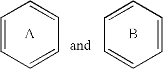
are each independently phenyl or pyridyl;
R₄ is hydrogen, cyano, alkyl, aryl, heteroaryl, alkylene-OH, alkylene-O-alkyl, —CO₂H, —CO₂-alkyl, alkylene-CO₂H, or alkylene-CO₂-alkyl, alkylene-OC(O)R wherein R is hydrogen or alkyl; cycloalkyl, heterocycloalkyl, alkylene-NH₂, alkylene-alkylamino, or alkylene-dialkylamino, any of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;
R₅ and R₆ are each independently selected from the group consisting of hydrogen, alkyl, alkylene-OH, aryl, alkylene-O-alkyl, —CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)—Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or
R₅and R₆, together with the nitrogen to which they are attached, may form a 3, 4, 5, or 6-membered saturated or unsaturated ring, optionally containing 1 or 2 additional heteroatoms selected from O, S, NH, or N-alkyl, and optionally substituted on carbon with halogen, alkyl, hydroxyl, or alkoxy;
R₇ is selected from the group consisting of alkyl, —OH, —O-alkyl, —CO₂H, —C(O)O-alkyl, —C(O)O-aryl, —CH₂═CHCO₂H, —CH₂═CHC(O)O-alkyl, —CH₂═CHC(O)O -aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, —OPO₂(S)NRₚ₁Rₚ₂, and —C(Z′)(Z″)PO₃Rₚ₁Rₚ₂, alkylene-OH, alkylene-CO₂H, alkylene-C(O)O-alkyl, alkylene-C(O)O-aryl, alkylene-CH═CHCO₂H, alkylene-CH₂═CHC(O)O-alkyl, alkylene-CH₂═CHC(O)O-aryl, alkylene-OPO₂Rₚ₁Rₚ₂, -alkylene-OPO₃Rₚ₁Rₚ₂, alkylene-PO₃Rₚ₁Rₚ₂, alkylene-OPO₂(S)Rₚ₁Rₚ₂, and alkylene-C(Z′)(Z″)PO₃Rₚ₁Rₚ₂, any of which may be optionally substituted on carbon with 1, 2, or 3 groups selected from halogen, alkyl, hydroxyl, carboxy, or alkoxy, or any 2 groups on the same carbon may be taken together to from C═O; and wherein
Z′ is hydroxyl or halogen;
Z″ is H or halogen;
Rₚ₁ and Rₚ₂ at each occurrence are independently hydrogen, C₁-C₆-alkyl, or aryl;
X is CRₓ₁Rₓ₂, NRₓ₃, —(CRₓ₁Rₓ₂)ₙNRₓ₃—, —NRₓ₃(CRₓ₁Rₓ₂)ₙ—, —O—, —S—, —(CRₓ₁Rₓ₂)ₙS—, —S(CRₓ₁Rₓ₂)ₙ—, —S(O)—, —(CRₓ₁Rₓ₂)ₙS(O)—, —S(O)(CRₓ₁Rₓ₂)ₙ—, —S(O)₂—, —(CRₓ₁Rₓ₂)ₙS(O)₂—, —S(O)₂(CRₓ₁Rₓ₂)ₙ—, —C(O)—, —(CRₓ₁Rₓ₂)ₙC(O)—, —C(O)(CRₓ₁Rₓ₂)ₙ—, —C(O)O—, —(CRₓ₁Rₓ₂)ₙC(O)O—, and —C(O)O(CRₓ₁Rₓ₂)ₙ—; wherein
Rₓ₁ and Rₓ₂ at each occurrence are independently selected from the group consisting of hydrogen, hydroxyl, halogen, alkyl, —O-alkyl, alkylyele-O-alkyl, alkyl-SO₂, CO₂H, and CO₂-alkyl, any of which may be optionally substituted on carbon with halogen; or taken together Rₓ₁ and Rₓ₂ may form a 3, 4, 5, or 6 membered ring optionally containing 1 or 2 heteroatoms selected from O, S, NH, or N-alkyl, which may itself be substituted on carbon with halogen, hydroxyl, or alkyl;
Rₓ₃ at each occurrence is selected from the group consisting of hydrogen and alkyl;
n is an integer from 0 to 4;
Y is selected from the group consisting of heterocyclo or heteroaryl —CR_{y1}R_{y2}, —CR_{y1}R_{y2}—NR_{y3}—, —NR_{y3}(CO)—, —(CO)—, —O—, —S—, —SO—, —SO₂—, —CR_{y1}R_{y2}—S—, —CR_{y1}R_{y2}—O—, —COO—, and —NR_{y3}SO₂—; wherein
R_{y1}, R_{y2}, R_{y3} at each occurrence are hydrogen or alkyl which may be substituted on carbon with halogen, hydroxyl, or alkyl; or
R_{y3} or —CR_{y1}R_{y2} and one of R₅ or R₆, together with the nitrogens to which they are attached, form a 5, 6, or 7-membered ring, optionally substituted on carbon with halogen, hydroxyl, or alkyl; and
R₈ₐ and R_{8b} are each independently hydrogen, halogen, alkyl, or taken together with the carbon to which they are attached, may form a 3, 4, 5, or 6-membered ring, optionally containing 1 or 2 heteroatoms selected from NH, N-alkyl, O, or S, and optionally substituted on carbon with halogen, or alkyl.

2. The compound of claim 1, wherein R₁ is benzyloxy.

3. The compound of claim 1, wherein Y is selected from the group consisting of

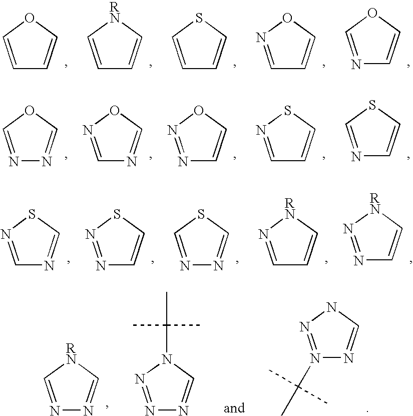

4. The compound of claim 1, wherein Y is selected from the group consisting of CH₂, CH₂NH, Y is NH(CO), Y is NMe(CO) and C═O.

5. A compound of formula II 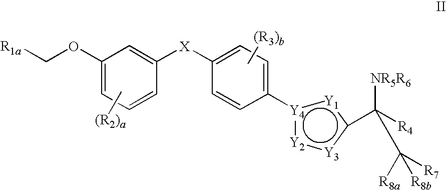
or a pharmaceutically acceptable salt thereof, wherein:
R₁ₐ is aryl or heteroaryl, either of which may be optionally substituted on carbon with 1, 2, or 3 groups selected from halo, alkyl, hydroxyl, or —O-alkyl;
R₂ is halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylSO₂, alkylSO, aralkylSO₂, aralkylSO, alkylene-CO-amino, alkylene-CO-alkylamino, alkylene-CO-dialkylamino, alkylene-NH—CO₂H, alkylene-NH—CO₂alkyl-CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;
R₃ is halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl-CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;
a is 0, 1, 2, or 3;
b is 1, 2 or 3; 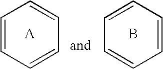
are each independently phenyl or pyridyl;
R₄ is hydrogen, cyano, alkyl, aryl, heteroaryl, alkylene-OH, aryl, alkylene-O-alkyl, —CO₂H, —CO₂-alkyl, alkylene-CO₂H, or alkylene-CO₂-alkyl, alkylene-OC(O)R wherein R is hydrogen or alkyl; cycloalkyl, heterocycloalkyl, alkylene-NH₂, alkylene-alkylamino, or alkylene-dialkylamino, any of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;
R₅and R₆ are each independently selected from the group consisting of hydrogen, alkyl, alkylene-OH, aryl, alkylene-O-alkyl, —CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)—Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or
R₅ and R₆, together with the nitrogen to which they are attached, may form a 3, 4, 5, or 6-membered saturated or unsaturated ring, optionally containing 1 or 2 additional heteroatoms selected from O, S, NH, or N-alkyl, and optionally substituted on carbon with halogen, alkyl, hydroxyl, or alkoxy;
R₇ is selected from the group consisting of alkyl, —OH, —O-alkyl, —CO₂H, —C(O)O-alkyl, —C(O)O-aryl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, —OPO₂(S)NRₚ₁Rₚ₂, and —C(Z′)(Z″)PO₃Rₚ₁Rₚ₂, alkylene-OH, alkylene-CO₂H, alkylene-C(O)O-alkyl, alkylene-C(O)O-aryl, alkylene-CH═CHCO₂H, alkylene-CH₂═CHC(O)O-alkyl, alkylene-CH₂═CHC(O)O-aryl, alkylene-OPO₂Rₚ₁Rₚ₂, -alkylene-OPO₃Rₚ₁Rₚ₂, alkylene-PO₃Rₚ₁Rₚ₂, alkylene-OPO₂(S)Rₚ₁Rₚ₂, and alkylene-C(Z′)(Z″)PO₃Rₚ₁Rₚ₂, any of which may be optionally substituted on carbon with 1, 2, or 3 groups selected from halogen, alkyl, hydroxyl, carboxy, or alkoxy, or any 2 groups on the same carbon may be taken together to from C═O; and wherein
Z′ is hydroxyl or halogen;
Z″ is H or halogen;
Rₚ₁ and Rₚ₂ at each occurrence are independently hydrogen, C₁-C₆-alkyl, or aryl;
X is CRₓ₁Rₓ₂, NRₓ₃, —(CRₓ₁Rₓ₂)ₙNRₓ₃—, —NRₓ₃(CRₓ₁Rₓ₂)ₙ—, —O—, —S—, —(CRₓ₁Rₓ₂)ₙS—, —S(CRₓ₁Rₓ₂)ₙ—, —S(O)—, —(CRₓ₁Rₓ₂)ₙS(O)—, —S(O)(CRₓ₁Rₓ₂)ₙ—, —S(O)₂—, —(CRₓ₁Rₓ₂)ₙS(O)₂—, —S(O)₂(CRₓ₁Rₓ₂)ₙ—, —C(O)—, —(CRₓ₁Rₓ₂)ₙC(O)—, —C(O)(CRₓ₁Rₓ₂)ₙ—, —C(O)O—, —(CRₓ₁Rₓ₂)ₙC(O)O—, and —C(O)O(CRₓ₁Rₓ₂)ₙ—; wherein
Rₓ₁ and Rₓ₂ at each occurrence are independently selected from the group consisting of hydrogen, hydroxyl, halogen, alkyl, —O-alkyl, alkylyene-O-alkyl, alkyl-SO₂, CO₂H, and CO₂-alkyl, any of which may be optionally substituted on carbon with halogen; or taken together Rₓ₁ and Rₓ₂ may form a 3, 4, 5, or 6 membered ring optionally containing 1 or 2 heteroatoms selected from O, S, NH, or N-alkyl, which may itself be substituted on carbon with halogen, hydroxyl, or alkyl;
Rₓ₃ at each occurrence is selected from the group consisting of hydrogen and alkyl;
n is an integer from 0 to 4; and 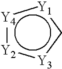
is a heteroaryl ring containing up to four heteroatoms selected from N, O, or S, optionally substituted on carbon with halogen or alkyl, wherein
Y₁ is N, S, or O;
Y₂ and Y₃ are each independently C, N, O, or S; provided that when 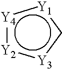
contains an N—H, that hydrogen may be replaced with alkyl;
Y₄ is C or N; and
R₈ₐ and R_{8b} are each independently hydrogen, halogen, alkyl, or taken together with the carbon to which they are attached, may form a 3, 4, 5, or 6-membered ring, optionally containing 1 or 2 heteroatoms selected from NH, N-alkyl, O, or S, and optionally substituted on carbon with halogen, or alkyl

6. A compound of formula III: 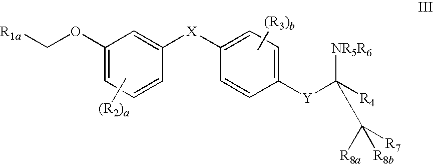
or a pharmaceutically acceptable salt thereof, wherein:
R₁ₐ is aryl or heteroaryl, either of which may be optionally substituted on carbon with 1, 2, or 3 groups selected from halo, alkyl, hydroxyl, or —O-alkyl;
R₂ is halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylSO₂, alkylSO, aralkylSO₂, aralkylSO, alkylene-CO-amino, alkylene-CO-alkylamino, alkylene-CO-dialkylamino, alkylene-NH—CO₂H, alkylene-NH—CO₂alkyl-CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;
R₃ is halogen, cyano, alkyl, aryl, heteroaryl, cycloalkyl, heterocycloalkyl, aralkyl, heteroalykl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl-CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;
a is 0, 1, 2, or 3;
b is 1, 2, or 3; 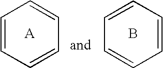
are each independently phenyl or pyridyl;
R₄ is hydrogen, cyano, alkyl, aryl, heteroaryl, alkylene-OH, aryl, alkylene-O-alkyl, —CO₂H, —CO₂-alkyl, alkylene-CO₂H, or alkylene-CO₂-alkyl, alkylene-OC(O)R wherein R is hydrogen or alkyl; cycloalkyl, heterocycloalkyl, alkylene-NH₂, alkylene-alkylamino, or alkylene-dialkylamino, any of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;
R₅ and R₆ are each independently selected from the group consisting of hydrogen, alkyl, alkylene-OH, aryl, alkylene-O-alkyl, —CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)—Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or
R₅and R₆, together with the nitrogen to which they are attached, may form a 3, 4, 5, or 6-membered saturated or unsaturated ring, optionally containing 1 or 2 additional heteroatoms selected from O, S, NH, or N-alkyl, and optionally substituted on carbon with halogen, alkyl, hydroxyl, or alkoxy;
R₇ is selected from the group consisting of alkyl, —OH, —O-alkyl, —CO₂H, —C(O)O-alkyl, —C(O)O-aryl, —CH₂═CHCO₂H, —CH₂═CHC(O)O-alkyl, —CH₂═CHC(O)O-aryl, —OPO₂Rₚ₁Rₚ₂, —OPO₃Rₚ₁Rₚ₂, —CH₂PO₃Rₚ₁Rₚ₂, —OPO₂(S)Rₚ₁Rₚ₂, —OPO₂(S)NRₚ₁Rₚ₂, and —C(Z′)(Z″)PO₃Rₚ₁Rₚ₂, alkylene-OH, alkylene-CO₂H, alkylene-C(O)O-alkyl, alkylene-C(O)O-aryl, alkylene-CH═CHCO₂H, alkylene-CH₂═CHC(O)O-alkyl, alkylene-CH₂═CHC(O)O-aryl, alkylene-OPO₂Rₚ₁Rₚ₂, -alkylene-OPO₃Rₚ₁Rₚ₂, alkylene-PO₃Rₚ₁Rₚ₂, alkylene-OPO₂(S)Rₚ₁Rₚ₂, and alkylene-C(Z′)(Z″)PO₃Rₚ₁Rₚ₂, any of which may be optionally substituted on carbon with 1, 2, or 3 groups selected from halogen, alkyl, hydroxyl, carboxy, or alkoxy, or any 2 groups on the same carbon may be taken together to from C═O; and wherein
Z′ is hydroxyl or halogen;
Z″ is H or halogen;
Rₚ₁ and Rₚ₂ at each occurrence are independently hydrogen, C₁-C₆-alkyl, or aryl;
X is CRₓ₁Rₓ₂, NRₓ₃, —(CRₓ₁Rₓ₂)ₙNRₓ₃—, —NRₓ₃(CRₓ₁Rₓ₂)ₙ—, —O—, —S—, —(CRₓ₁Rₓ₂)ₙS—, —S(CRₓ₁Rₓ₂)ₙ—, —S(O)—, —(CRₓ₁Rₓ₂)ₙS(O)—, —S(O)(CRₓ₁Rₓ₂)ₙ—, —S(O)₂—, —(CRₓ₁Rₓ₂)ₙS(O)₂—, —S(O)₂(CRₓ₁Rₓ₂)ₙ—, —C(O)—, —(CRₓ₁Rₓ₂)ₙC(O)—, —C(O)(CRₓ₁Rₓ₂)ₙ—, —C(O)O—, —(CRₓ₁Rₓ₂)ₙC(O)O—, and —C(O)O(CRₓ₁Rₓ₂)ₙ—; wherein
Rₓ₁ and Rₓ₂ at each occurrence are independently selected from the group consisting of hydrogen, hydroxyl, halogen, alkyl, —O-alkyl, alkylyene-O-alkyl, alkyl-SO₂, CO₂H, and CO₂-alkyl, any of which may be optionally substituted on carbon with halogen; or taken together Rₓ₁ and Rₓ₂ may form a 3, 4, 5, or 6 membered ring optionally containing 1 or 2 heteroatoms selected from O, S, NH, or N-alkyl, which may itself be substituted on carbon with halogen, hydroxyl, or alkyl;
Rₓ₃ at each occurrence is selected from the group consisting of hydrogen and alkyl;
n is an integer from 0 to 4; and
Y is —CH₂NR′″—, —CH₂NR′″(CO)—, —CHFNR′″—, —CHFNR′″(CO)—, —CF₂NR′″—, —CF₂NR′″(CO)—, —CH₂(CO)—, —CHF(CO)—, —CF₂(CO)—, —(CO)CF₂—, —CH₂(CHOH)—, —CHF(CHOH)—, —CF₂(CHOH)—, —(CHOH)CF₂—, —NH(CO)—, —(CO)—, —(CO)₂—, —O—, —S—, —SO—, —SO₂—, —CH₂O—, —CH₂CH₂O—, —CH₂OCH₂—, —OCH₂O—, —CH₂S—, —CH₂SO—, —CH₂SO₂—, —CHFO—, —CHFS—, —CHFSO—, —CHFSO₂—, —CF₂O——CF₂S—, —CF₂SO—, —CF₂SO₂—, —NR′″SO₂—, —CF₂—, —CF₂CF₂—, —CF₂CF₂CF₂—, wherein R′″ is H or alkyl; and
R₈ₐ and R_{8b} are each independently hydrogen, halogen, alkyl, or taken together with the carbon to which they are attached, may form a 3, 4, 5, or 6-membered ring, optionally containing 1 or 2 heteroatoms selected from NH, N-alkyl, O, or S, and optionally substituted on carbon with halogen, or alkyl.

7. The compound of claim 1, wherein R₄ is selected from the group consisting of hydrogen, methyl and hydroxymethyl.

8. The compound of claim 1, wherein R₅and R₆ are each independently hydrogen.

9. The compound of claim 1, wherein R₇ is selected from the group consisting of OH, CO₂H, CO₂Me, CO₂Et, CO₂-phenyl and —OP(O)₃H₂.

10. The compound of claim 1, wherein X is selected from the group consisting of CH2, NH, N-alkyl, O, S, SO, SO₂ and CO.

11. The compound of claim 1, wherein, R₈ₐ and R_{8b} are each independently hydrogen.

12. A compound selected from the group consisting of: 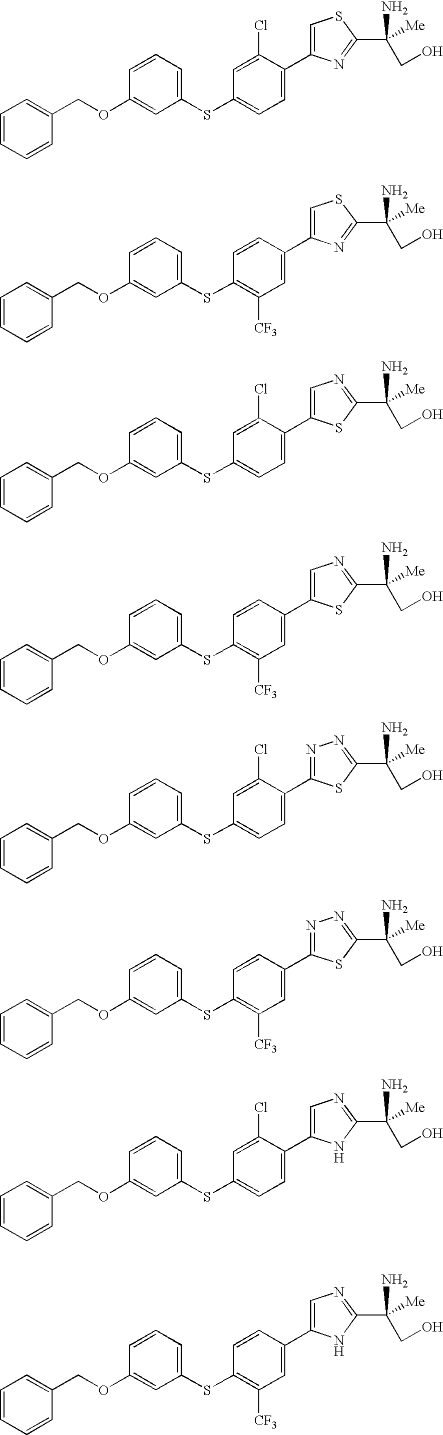 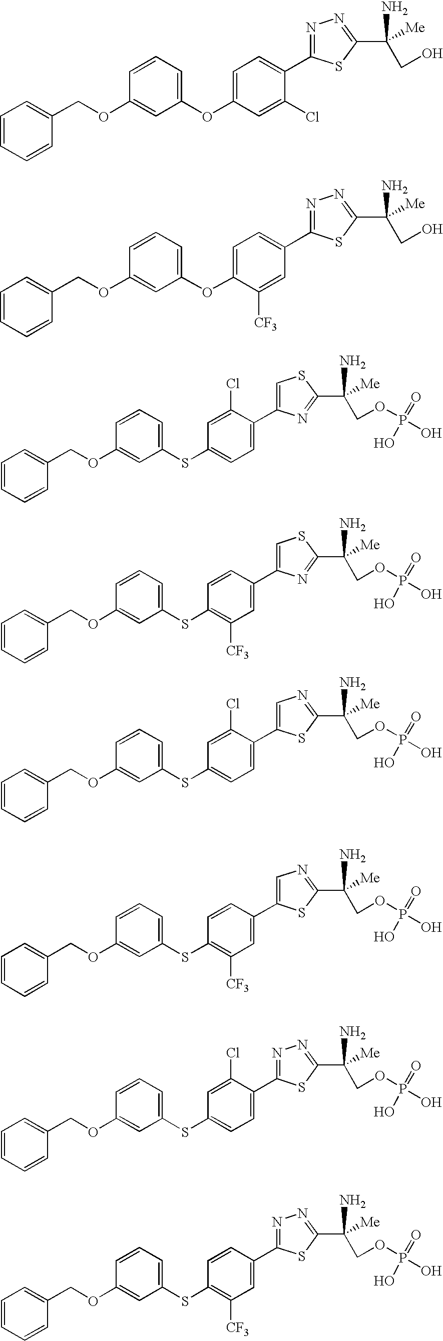 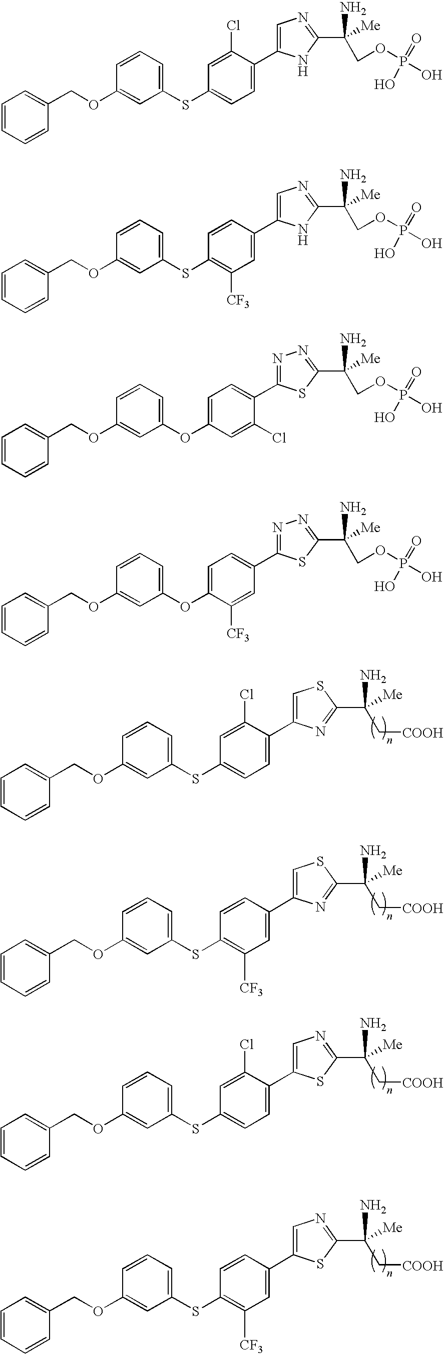 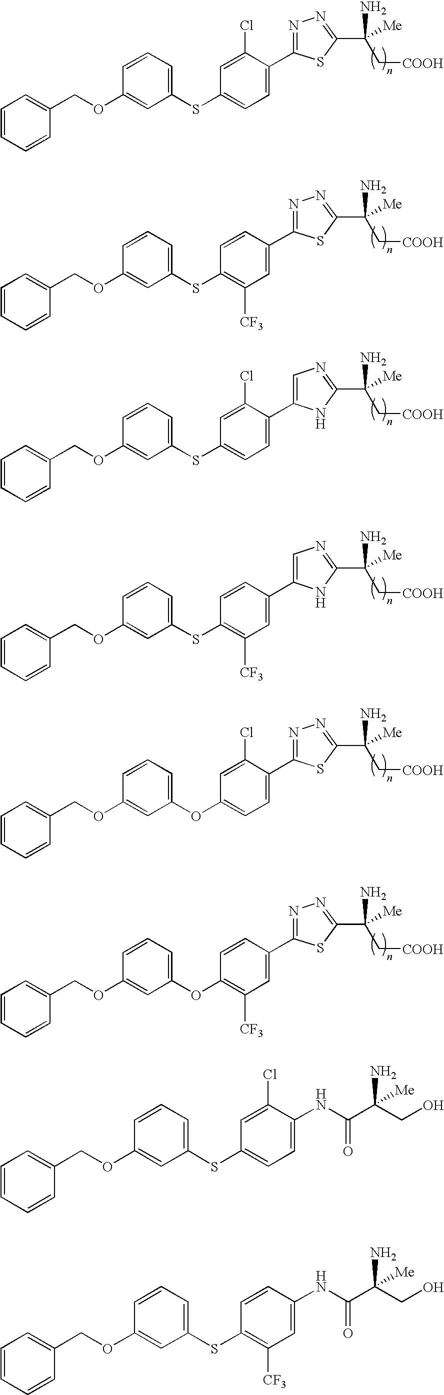 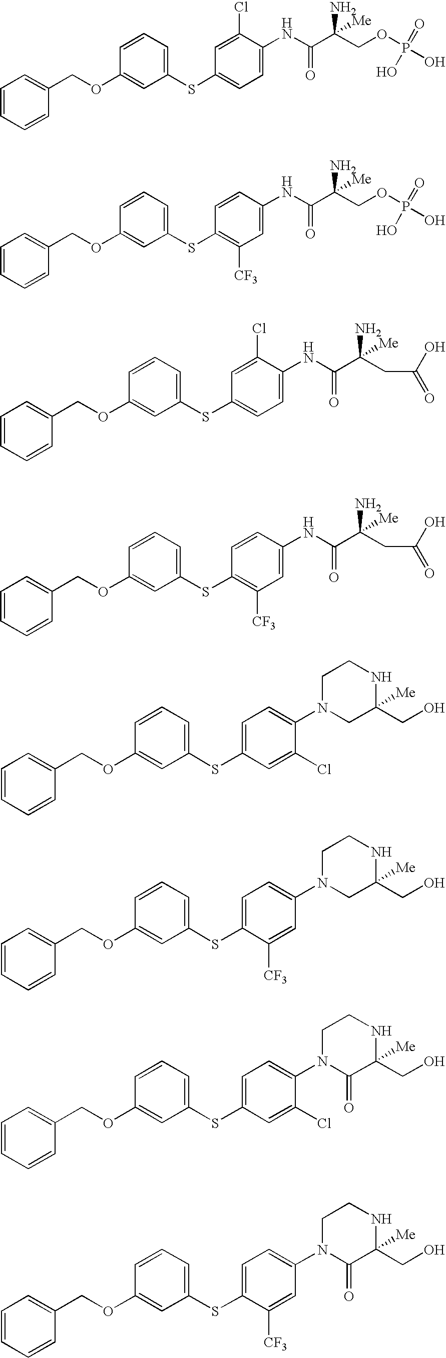 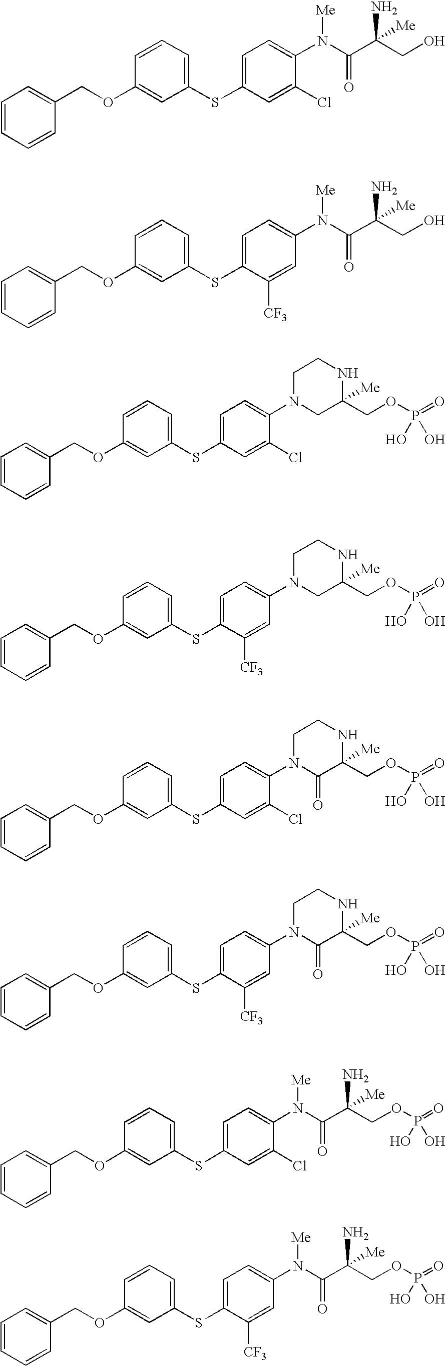 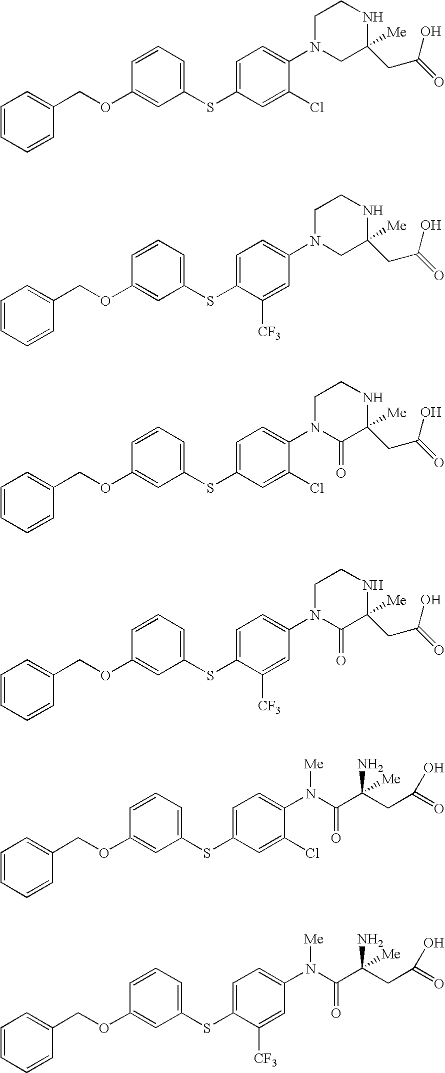
wherein n is 0, 1, or 2 for the above compounds,
and pharmaceutically acceptable salts, phosphate derivatives, phosphate mimics, or phosphate precursor analogs thereof.

13. The compound as defined in claim 1 or a pharmaceutically acceptable salt, phosphate derivative, phosphate mimic, or phosphate precursor analog thereof for use as a therapeutic substance.

14. The compound as defined in claim 1 or a pharmaceutically acceptable salt, phosphate derivative, phosphate mimic, or phosphate precursor analog thereof for use in the treatment of a sphingosine associated disorder.

15. The compound as defined in claim 1 or a pharmaceutically acceptable salt, phosphate derivative, phosphate mimic, or phosphate precursor analog thereof for use in the treatment of multiple sclerosis.

16. (canceled)

17. (canceled)

18. A method of treating a sphingosine 1-phosphate associated disorder comprising administering to a subject a therapeutically effective amount of a compound as defined in claim 1 or a pharmaceutically acceptable salt, phosphate derivative, phosphate mimic, or a phosphate precursor analog thereof.

19. A pharmaceutical composition comprising a compound as defined in claim 1 or a pharmaceutically acceptable salt, phosphate derivative, phosphate mimic, or a phosphate precursor analog thereof.

20. A process for the preparation of a pharmaceutical composition according to claim 19.

21. A process for the preparation of a compound as defined in claim 1 or a pharmaceutically acceptable salt, phosphate derivative, phosphate mimic, or phosphate precursor analog thereof.
